SEQUENCE LISTING (112 sequences)

SEQ ID NO: 1 - DNA, 1119 bp, Homo sapiens
atgttagccaacagctcctcaaccaacagttctgttctcccgtgtcctgactaccgacctacccaccgcctgcacttggtggtctacagcttggtgctggctgccgggctccccctcaacgcgctagccctctgggtcttcctgcgcgcgctgcgcgtgcactcggtggtgagcgtgtacatgtgtaacctggcggccagcgacctgctcttcaccctctcgctgcccgttcgtctctcctactacgcactgcaccactggcccttccccgacctcctgtgccagacgacgggcgccatcttccagatgaacatgtacggcagctgcatcttcctgatgctcatcaacgtggaccgctacgccgccatcgtgcacccgctgcgactgcgccacctgcggcggccccgcgtggcgcggctgctctgcctgggcgtgtgggcgctcatcctggtgtttgccgtgcccgccgcccgcgtgcacaggccctcgcgttgccgctaccgggacctcgaggtgcgcctatgcttcgagagcttcagcgacgagctgtggaaaggcaggctgctgcccctcgtgctgctggccgaggcgctgggcttcctgctgcccctggcggcggtggtctactcgtcgggccgagtcttctggacgctggcgcgccccgacgccacgcagagccagcggcggcggaagaccgtgcgcctcctgctggctaacctcgtcatcttcctgctgtgcttcgtgccctacaacagcacgctggcggtctacgggctgctgcggagcaagctggtggcggccagcgtgcctgcccgcgatcgcgtgcgcggggtgctgatggtgatggtgctgctggccggcgccaactgcgtgctggacccgctggtgtactactttagcgccgagggcttccgcaacaccctgcgcggcctgggcactccgcaccgggccaggacctcggccaccaacgggacgcgggcggcgctcgcgcaatccgaaaggtccgccgtcaccaccgacgccaccaggccggatgccgccagtcaggggctgctccgaccctccgactcccactctctgtcttccttcacacagtgtccccaggattccgccctctga

SEQ ID NO: 2 - protein, 372 aa, Homo sapiens [sequence omitted]

SEQ ID NO: 3 - DNA, 3379 bp, Homo sapiens
gcgtccgaaaaaaaaagaaattcctttacatactacaacatgaatagatcttggaaacattatgctaagtgaaataaaccagacacaaaaggacaaatattgtatgattccactcatatgaggtatctagaataggcaaattcattgagacagaaagtagactagaaccagaagctgaatggggtgcggtgggtagtactgcttaatgactgcagagttgttgcttggttgatgaaaaagttctatttctggaaacagagagtggtgacggttaagcaacactgtcttggtcttttttgttgttgttgttgtttttgagacggactctcactctgtctcccaggccggagtgcgatggattagacctgctaggggagcacttggcaaaactcaacccacagggccttcccctgcctagcaagactgtgctgtcaaatttattcacatgtggctctggtcaagactagcatgcaatcagcctatgagggcattattatattattattcccattttacagatgaagaaactgagaagtcaaaccattaagctgaacccagtttgctttgaccacaaatccagccctcacaggcgcagtgatgcatgtgatgcgtaaggctgggatgttgttctgtatttgggagttttgtttgcttgtttgcttgtctgacatggagtctcactctgtcacccaggctggagtgcagtggcgtgatctcggctcactgcaacctccgcctcccgggttcaaggactctcctgctgcagcctcccatgtactcaaagagtttgacctttattctttggataatgaggagctagcctagcacctggtccaaggaggtgctccataagaccacctattgatttgtgcttattatctgtctccctccaatggaatgtaaaggaggtgggggcaaagactttttgctttgttccctgctgtgaacatgcctggaactttctatgagctcagtaagcaaggaaagaaggaaggaagagatcttgagatagtaacagcaacctaagcgttttacacacgtcatcttaatctccaaacctcatgaattctctctctctctctctcattttttgagacagagtctcgctctgtcacccaggctggagtgcagtggcgtgatctcgactcattgcaacctctgcctcctggattcaatcaattctcatgccttagcctactgaggagctgggattacaagtgcacgccaccatacccggctaatctttgtatttttagtagaggcaagattttgtcatgttggccaggttggtcttcaactcctggcctcaagtaatccacccacatcagcctcccaaagtgctgagatcacaggcatgaggtaccatgcagccgcctttttttttttttttgagatggagtctcgttttgttacccaggctggagggcagtggtacgatgtcagctcactgcaacctccgcctcctgggttcaagtgattctcctgtgtgagcctcctgagtagctgggactacaggtgcatgccaccacatctggctaatttttgtatttttagtagagacagggttttgccaggttggccaggctgatctcgaactcctgacctcaggtgatctgcccgcctcagtctcccaaagtgctggattacaggtgtgggccactacgccggccctggccctctttctttcttttttgagatgggctcactctgtcacccaggcaggagtgcagtggtgggcttgaggctcactgcactgcagcctccacctccctggagtcaagtgattctctcacctcagcctcacaagtagctgggactacgggcatgtgccacaatgcctggctaattttttaattttttaatattttttattttatttttttttgagacagagtcttgctctgtcacccaggccggagtgcaatggtgtgatctcggctcactgcaacctctgctcaagcaattctccctgccttagcctcctgagtagctgggattacaggcgcctgccaccacgcccggctaatttttttttttttttagtagagacaggattttgccatgttggccaggatggtctcaacctcctgacctcaggtgatccgcccacctcagcctcccaaagtgctcggattacagatgtgagccaccacgcccagccttatttttattttttattttattttatttatttattttgagatggagtttcactcttgttgcccaggctggagtgcaatggcgcgatcttggctcactgcaaactccaccccccaggttcaagcaattctcctgtctcagccccctgagtagctgggattacaggcgcccgcccctatgccaggctaatttttggtattttttttagtagagatggggtttcaccatgttggccaagctggtctcgaactcctgacctcaggtgatccacctgcctcggcctcccaaagtgctgggattacaggcgtgagccaccgcgcctggctatttttattttttgagacagagtttcacttttgttgtccaggctggagtgcaatggcacagtctcagctcactgcaacctctgcctcctggtttcaagcgattctcctgtctcagcctcccgagtagctgggattacaggcgtgcaccaccacgcccagctaatttttgtatttttagtagagatggggtttcaccatattggacaggttggtctcgaactcctgacctcaggtaatccacccgcctcggcctcccaaaatgctgggattacaggtgtgagccactgcacctggccctgtatttttttgtagagatggggtttcgccgtgttgcccaggctggtccccaactcctaggttcaagcaattggtctgccttggcctcccaaagtgccaggattacaggtgtaagccattgcacccagccaagattaatttttttgaagtcacacaactaggcaagttagcaaaaccaagatttaaacctaggcatccgagtccctgccttcaaacctgggtgtttaacactatactatatagtcctgccgtaggaacctattctagcccaatggcagacttgaggctgagaaaagattcagaaggcctgccagtggagctaaacatttgtgtgtgcagccctgtctctgtataacttccggcttgccttcctattccaggtctctgctgctgatgaagctgtgaccaaacgcacccaacccttggcagccatctgtccctgcagccatagcccacattcccatgacctccctctgcttgttttgggaccatgtctgtacagcctctaggccccagccccggaggtgaatgccatgccatgattctggtgtgctccatggcatccccagcctagctcccaatcccactttggcacg

SEQ ID NO: 4 - DNA, 1302 bp, Homo sapiens
acacacatgccattgcgctgtccgtgcccgactcccaacgcctctcgttctgggaggcttacagggtgtacacacaagaaggtgggctgggcacttggacctttgggtggcaattccagcttagcaacgcagaagagtacaaagtgtggaagccagggcccagggaaggcagtgctgctggaaatggcttctttaaactgtgagcacgcagagcaccccttctccagcggtgggaagtgatgcagagagcccacccgtgcagagggcagaagaggacgaaatgcctttgggtgggcagggcattaaactgctaaaagctggttagatggaacagaaaatgggcattctggatctaaaccgccacaggggcctgagagctgaagagcaccaggtttggtggacaaagctactgagatgcctgttcatctgctgacttctgtctaggctcatggatgccaccccctttcatttcggcctaggcttcccctgctcaccactgaggcctaatacaagagttcctatggacagaactacattctttctcgcatagtgacttgtgacaatttagacttggcatccagcatgggatagttggggcaaggcaaaactaacttagagtttccccctcaacaacatccaagtccaaaccctttttaggttatcctttcttccatcacatccccttttccaggcctcctccattttaggtccttaatattctttctttttctctctctctcgtttctctcttctctctcctctcctctctcttctcctcttctctctctctccctctctcctttgtccagagtaaggataaaattctttctactaaagcactggttctcaaactttttggtctcagaccccactcttagaaattgaggatctcaaagagctttgcttatattttgttcttttgatacttaccatactagaaattaaagcgaatacatttttaaaataaatacacatgcacacattacattagccatgggagcaataatgtcaccacacacacttcatgaagcctctggaaaactctacagtatacttgtgagagaatgagagtgaaagggacaaataacatctgtgtagcagtattatgaaaatagcttgacctcgtggacttcctcagagggttggtccctggatcacactttgagaaccatacttgtcctgaagtattggagttcatgtctaacttcttcccagggcattatgtacagtgctttttattactgtggggagagggcagtgctaaataaattaatcactactgataaaaaaaaaaaaaaaaag

SEQ ID NO: 5 - DNA, 80 bp, Artificial
  oligonucleotide
ccaagctgtagaccaccaagtgcaggcggtgggtaggtcggtagtcaggacacgggagaacagaactgttggttgaggag

SEQ ID NO: 6 - DNA, 20 bp, Artificial
  oligonucleotide
agcccaatggcagacttgag

SEQ ID NO: 7 - DNA, 20 bp, artificial
  oligonucleotide
ggtgcgtttggtcacagctt

SEQ ID NO: 8 - protein, 365 aa, Homo sapiens
MGNITADNSSMSCTIDHTIHQTLAPVVYVTVLVVGFPANCLSLYFGYLQIKARNELGVYLCNLTVADLFYICSLPFWLQYVLQHDNWSHGDLSCQVCGILLYENIYISVGFLCCISVDRYLAVAHPFRFHQFRTLKAAVGVSVVIWAKELLTSIYFLMHEEVIEDENQHRVCFEHYPIQAWQRAINYYRFLVGFLFPICLLLASYQGILRAVRRSHGTQKSRKDQIQRLVLSTVVIFLACFLPYHVLLLVRSVWEASCDFAKGVFNAYHFSLLLTSFNCVADPVLYCFVSETTHRDLARLRGACLAFLTCSRTGRAREAYPLGAPEASGKSGAQGEEPELLTKLHPAFQTPNSPGSGGFPTGRLA

SEQ ID NO: 9 - protein, 361 aa, Homo sapiens
MGNITADNTSMNCDIDHTIHQTLAPVVYVMVLVVGFPANCLSLYYGYLQIKARNELGVYLCNLTVADLFYICSLPFWLQYVLQHDHWSHDDLSCQVCGILLYENIYISVGFLCCISIDRYLAVAHPFRFHQFRTLKAAMGVSALIWVKELLTSIYFLMHEEVVEDADRHRVCFEHYPLEPRQRGINYYRFLVGFLFPICLLLASYRGILRAVRRSHGTQKSRKDQIQRLVLSTVVIFLACFLPYHVLLLVRSLWESSCDFAKGIFNAYHFSLLLTSFNCVADPVLYCFVSETTHRDLARLRGACLAFLTCARTGRAREAYPLGAPEASGKSEDPEVLTRLHPAFQTPHPPGMGGSPAGGLS

SEQ ID NO: 10 - protein, 368 aa, Homo sapiens
MGDRRFIDFQFQDSNSSLRPRLGNATANNTCIVDDSFKYNLNGAVYSVVFILGLITNSVSLFVFCFRMKMRSETAIFITNLAVSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTVCFEHYPLEPRQRGINYYRFLVGFLFPICLLLASYRGILRAVRRSHGTLSQIGTNKKKVLKMITVHMAVFVVCFVPYNSVLFLYALVRSQAITNCFLERFAKIMYPITLCLATLNCCFDPFIYYFTLESFQKSFYINAHIRMESLFKTETPLTTKPSLPAIQEEVSDQTTNNGGELMLESTF

SEQ ID NO: 11 - protein, 370 aa, Homo sapiens
MGDRRFIDFQFQDSNSSLRPRLGNATANNTCIVDDSFKYNLNGAVYSVVFILGLITNSVSLFVFCFRMKMRSETAIFITNLAVSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTTCFEGFSKRVWKTYLSKITIFIEVVGFIIPLILNVSCSSVVLRTLRKPATLSQIGTNKKKVLKMITVHMAVFVVCFVPYNSVLFLYALVRSQAITNCFLERFAKIMYPITLCLATLNCCFDPFIYYFTLESFQKSFYINAHIRMESLFKTETPLTTKPSLPAIQEEVSDQTTNNGGELMLESTF

SEQ ID NO: 12 - protein, 308 aa, chicken
MVSSNCSTEDSFKYTLYGCVFSMVFVLGLIANCVAIYIFTFTLKVRNETTTYMLNLAISDLLFVFTLPFRIYYFVVRNWPFGDVLCKISVTLFYTNMYGSILFLTCISVDRFLAIVHPFRSKTLRTKRNARIVCVAVWITVLAGSTPASFFQSTNRQNNTEQRTCFENFPESTWKTYLSRIVIFIEIVGFFIPLILNVTCSTMVLRTLNKPLTLSRNKLSKKKVLKMIFVHLVIFCFCFVPYNITLILYSLMRTQTWINCSVVTAVRTMYPVTLCIAVSNCCFDPIVYYFTSDTNSELDKKQQVHQNT

SEQ ID NO: 13 - protein, 344 aa, Homo sapiens
MVSVNSSHCFYNDSFKYTLYGCMFSMVFVLGLVSNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 14 - protein, 339 aa, Homo sapiens
MNGLEVAPPGLITNFSLATAEQCGQETPLENMLFASFYLLDFILALVGNTLALWLFIRDHKSGTPANVFLMHLAVADLSCVLVLPTRLVYHFSGNHWPFGEIACRLTGFLFYLNMYASIYFLTCISADRFLAIVHPVKSLKLRRPLYAHLACAFLWVVVAVAMAPLLVSPQTVQTNHTVVCLQLYREKASHHALVSLAVAFTFPFITTVTCYLLIIRSLRQGLRVEKRLKTKAVRMIAIVLAIFLVCFVPYHVNRSVYVLHYRSHGASCATQRILALANRITSCLTSLNGALDPIMYFFVAEKFRHALCNLLCGKRLKGPPPSFEGKTNESSLSAKSEL

SEQ ID NO: 15 - protein, 361 aa, rat
MTSAESLLFTSLGPSPSSGDGDCRFNEEFKFILLPMSYAVVFVLGLALNAPTLWLFLFRLRPWDATATYMFHLALSDTLYVLSLPTLVYYYAARNHWPFGTGLCKFVRFLFYWNLYCSVLFLTCISVHRYLGICHPLRAIRWGRPRFASLLCLGVWLVVAGCLVPNLFFVTTNANGTTILCHDTTLPEEFDHYVYFSSAVMVLLFGLPFLITLVCYGLMARRLYRPLPGAGQSSSRLRSLRTIAVVLTVFAVCFVPFHITRTIYYQARLLQADCHVLNIVNVVYKVTRPLASANSCLDPVLYLFTGDKYRNQLQQLCRGSKPKPRTAASSLALVTLHEESISRWADTHQDSTFSAYEGDRL

SEQ ID NO: 16 - protein, 388 aa, Homo sapiens [sequence omitted]

SEQ ID NO: 17 - protein, 12 aa, Artificial
  peptide
LEAKIWVVPAPS

SEQ ID NO: 18 - protein, 12 aa, Artificial
  peptide
TGQTKIWYPHST

SEQ ID NO: 19 - protein, 12 aa, Artificial
  peptide
VYSKVWLLPAGQ

SEQ ID NO: 20 - protein, 12 aa, Artificial Sequence
HLKVWEVRSPGP

SEQ ID NO: 21 - protein, 12 aa, Artificial
  peptide
NAKVWTVPSKPP

SEQ ID NO: 22 - protein, 12 aa, Artificial
  peptide
KVWIPTSTWLQT

SEQ ID NO: 23 - protein, 12 aa, Artificial
  peptide
KVWSLDISAPQH

SEQ ID NO: 24 - protein, 15 aa, Artificial
  peptide
ADVLHATPSEKVWLL

SEQ ID NO: 25 - protein, 15 aa, Artificial
  peptide
KVVDSNHKVWLVSQT

SEQ ID NO: 26 - protein, 12 aa, Artificial
  peptide
NHDNTKKVWILA

SEQ ID NO: 27 - protein, 9 aa, Artificial
  peptide
TPHRVWXLP

SEQ ID NO: 28 - DNA, 23 bp, Artificial
  oligonucleotide
cctggcttccacactttgtactc

SEQ ID NO: 29 - DNA, 19 bp, Artificial
  oligonucleotide
tcccaacgcctctcgttct

SEQ ID NO: 30 - DNA, 17 bp, Artificial
  oligonucleotide
agccgagccacatcgct

SEQ ID NO: 31 - DNA, 19 bp, Artificial
  oligonucleotide
gtgaccaggcgcccaatac

SEQ ID NO: 32 - DNA, 28 bp, Artificial
  oligonucleotide
caaatccgttgactccgaccttcacctt

SEQ ID NO: 33 - DNA, 50 bp, Artificial
  oligonucleotide
gtccccaagcttgcaccatgttagccaacagctcctcaaccaacagttct

SEQ ID NO: 34 - DNA, 72 bp, Artificial
  oligonucleotide
gtccgcggatccctacttgtcgtcgtcgtccttgtagtccatgagggcggaatcctggggacactgtgtgaa

SEQ ID NO: 35 - protein, 12 aa, Artificial
  peptide
KLWILADNFTNR

SEQ ID NO: 36 - protein, 15 aa, Artificial
  peptide
INSPHELKKLWLLPP

SEQ ID NO: 37 - protein, 12 aa, Artificial
  peptide
FPHKLWVLPVKT

SEQ ID NO: 38 - protein, 12 aa, Artificial
  peptide
KLWTIPSNDYPP

SEQ ID NO: 39 - protein, 12 aa, Artificial
  peptide
KLWELYPTVPAG

SEQ ID NO: 40 - protein, 12 aa, Artificial
  peptide
KLWIPHTSQPFL

SEQ ID NO: 41 - protein, 12 aa, Artificial
  peptide
KLWDITAPLPKP

SEQ ID NO: 42 - protein, 12 aa, Artificial
  peptide
NAKLWQIPAIPH

SEQ ID NO: 43 - protein, 12 aa, Artificial
  peptide
KLWVPQNRPELV

SEQ ID NO: 44 - protein, 12 aa, Artificial
  peptide
KLWELYPTVPAG

SEQ ID NO: 45 - protein, 12 aa, Artificial
  peptide
TSTPHRVWQLPV

SEQ ID NO: 46 - protein, 15 aa, Artificial
  peptide
TTPHRVWNLPLEAQQ

SEQ ID NO: 47 - DNA, 36 bp, Artificial
  oligonucleotide
ttggaggcgaagatttgggtggtgcctgcgccttct

SEQ ID NO: 48 - DNA, 36 bp, Artificial
  oligonucleotide
actgggcagactaagatttggtatccgcattctacg

SEQ ID NO: 49 - DNA, 36 bp, Artificial
  oligonucleotide
gtttattcgaaggtttggctgcttccggcgggtcag

SEQ ID NO: 50 - DNA, 36 bp, Artificial
  oligonucleotide
catcttaaggtgtgggaggttcggtcgcctgggcct

SEQ ID NO: 51 - DNA, 36 bp, Artificial
  oligonucleotide
aatgcgaaggtgtggacggttccgtcgaagccgcct

SEQ ID NO: 52 - DNA, 36 bp, Artificial
  oligonucleotide
aaggtgtggattcctacgagtacttggctgcagact

SEQ ID NO: 53 - DNA, 36 bp, Artificial
  oligonucleotide
aaggtttggagtttggatatttcggctccgcagcat

SEQ ID NO: 54 - DNA, 45 bp, Artificial
  oligonucleotide
gcggatgtgttgcatgcatacccctctgagaaggtctggcttctg

SEQ ID NO: 55 - DNA, 45 bp, Artificial
  oligonucleotide
aaggtggtggatagtaatcataaggtttggctggtttctcagact

SEQ ID NO: 56 - DNA, 36 bp, Artificial
  oligonucleotide
aatcatgataatactaagaaggtttggattctggct

SEQ ID NO: 57 - DNA, 36 bp, Artificial
  oligonucleotide
aagctttggattctggctgataattttacgaatcgg

SEQ ID NO: 58 - DNA, 45 bp, Artificial
  oligonucleotide
attaattctccgcatgaacttaagaagctgtggcttctgccgcct

SEQ ID NO: 59 - DNA, 36 bp, Artificial
  oligonucleotide
tttccgcataagttgtgggttttgccggtgaagact

SEQ ID NO: 60 - DNA, 36 bp, Artificial
  oligonucleotide
aagctgtggacgattcctagtaatgattatccgcct

SEQ ID NO: 61 - DNA, 36 bp, Artificial
  oligonucleotide
aagctttgggagttgtatccgactgtgccggctggt

SEQ ID NO: 62 - DNA, 36 bp, Artificial
  oligonucleotide
aagctgtggattcctcatacttctcagccgtttctt

SEQ ID NO: 63 - DNA, 36 bp, Artificial
  oligonucleotide
aagttgtgggatattacggctcctttgcctaagcct

SEQ ID NO: 64 - DNA, 18 bp, Artificial
  oligonucleotide
ccagcggtgggaagtgat

SEQ ID NO: 65 - DNA, 22 bp, Artificial
  oligonucleotide
caaaggcatttcgtcctcttct

SEQ ID NO: 66 - DNA, 21 bp, Artificial
  oligonucleotide
ccctctgcacgggtgggctct

SEQ ID NO: 67 - DNA, 36 bp, Artificial
  oligonucleotide
aatgcgaagctttggtagattcctgcgattccgcat

SEQ ID NO: 68 - DNA, 36 bp, Artificial
  oligonucleotide
aagctttgggttccgcagaatcgtccggagctggtg

SEQ ID NO: 69 - DNA, 36 bp, Artificial; identical to SEQ ID NO: 61
  oligonucleotide

SEQ ID NO: 70 - DNA, 36 bp, Artificial
  oligonucleotide
acttctactcctcatagggtttggcagctgcctgtt

SEQ ID NO: 71 - DNA, 45 bp, Artificial
  oligonucleotide
actactcctcatcgtgtatggaacctgcccctggaggctcagcag

SEQ ID NO: 72 - DNA, 26 bp, Artificial
  oligonucleotide
gataaaccgatacaattaaaggctcc

SEQ ID NO: 73 - protein, 15 aa, Artificial
  peptide
THGFGHRVWSVPLRS

SEQ ID NO: 74 - protein, 15 aa, Artificial
  peptide
SRVSGAKVWFLSNWS

SEQ ID NO: 75 - protein, 12 aa, Artificial
  peptide
AMNSHKIWMLPH

SEQ ID NO: 76 - protein, 12 aa, Artificial
  peptide
GLKIWSLPPHHG

SEQ ID NO: 77 - protein, 12 aa, Artificial
  peptide
KVWQMAPTTAFS

SEQ ID NO: 78 - DNA, 36 bp, Artificial
  oligonucleotide
ctngargcnaarathtgggtngtnccngcnccntcn

SEQ ID NO: 79 - DNA, 36 bp, Artificial
  oligonucleotide
gcnggncaracnaarathtggtayccncaytcnacn

SEQ ID NO: 80 - DNA, 36 bp, Artificial
  oligonucleotide
gtntaytcnaargtntggctnctnccngcnggncar

SEQ ID NO: 81 - DNA, 36 bp, Artificial
  oligonucleotide
cayctnaargtntgggargtncgntcnccnggnccn

SEQ ID NO: 82 - DNA, 36 bp, Artificial
  oligonucleotide
aaygcnaargtntggacngtnccntcnaarccnccn

SEQ ID NO: 83 - DNA, 36 bp, Artificial
  oligonucleotide
aargtntggathccnacntcnacntggctncaracn

SEQ ID NO: 84 - DNA, 36 bp, Artificial
  oligonucleotide
aargtntggtcnctngayathtcngcnccncarcay

SEQ ID NO: 85 - DNA, 45 bp, Artificial
  oligonucleotide
gcngaygtnctncaygcnacnccntcngaraargtntggctnctn

SEQ ID NO: 86 - DNA, 45 bp, Artificial
  oligonucleotide
aargtngtngaytcnaaycayaargtntggctngtntcncaracn

SEQ ID NO: 87 - DNA, 36 bp, Artificial
  oligonucleotide
aaycaygayaayacnaaraargtntggathctngcn

SEQ ID NO: 88 - DNA, 39 bp, Artificial
  oligonucleotide
aarctnttrtggathctngcngayaayttyacnaaycgn

SEQ ID NO: 89 - DNA, 45 bp, Artificial
  oligonucleotide
athaaytcnccncaygarctnaaraarctntggctnctnccnccn

SEQ ID NO: 90 - DNA, 36 bp, Artificial
  oligonucleotide
ttyccncayaarctntgggtnctnccngtnaaracn

SEQ ID NO: 91 - DNA, 36 bp, Artificial
  oligonucleotide
aarctntggacnathccntcnaaygaytayccnccn

SEQ ID NO: 92 - DNA, 36 bp, Artificial
  oligonucleotide
aarctntgggarctntayccnacngtnccngcnggn

SEQ ID NO: 93 - DNA, 36 bp, Artificial
  oligonucleotide
aarctntggathccncayacntcncarccnttyctn

SEQ ID NO: 94 - DNA, 36 bp, Artificial
  oligonucleotide
aarctntgggayathacngcnccnctnccnaarccn

SEQ ID NO: 95 - DNA, 36 bp, Artificial
  oligonucleotide
aaygcnaarctntggcarathccngcnathccncay

SEQ ID NO: 96 - DNA, 36 bp, Artificial
  oligonucleotide
aarctntgggtnccncaraaycgnccngarctngtn

SEQ ID NO: 97 - DNA, 36 bp, Artificial; identical to SEQ ID NO: 92
  oligonucleotide

SEQ ID NO: 98 - DNA, 36 bp, Artificial
  oligonucleotide
acntcnacnccncaycgngtntggcarctnccngtn

SEQ ID NO: 99 - DNA, 45 bp, Artificial
  oligonucleotide
acnacnccncaycgngtntggaayctnccnctngargcncarcar

SEQ ID NO: 100 - DNA, 45 bp, Artificial
  oligonucleotide
actcatggttttggtcatcgtgtgtggagtgttccgttgcgttcg

SEQ ID NO: 101 - DNA, 45 bp, Artificial
  oligonucleotide
agtagggtgtctggtgcgaaggtttggtttttgagtaattggtct

SEQ ID NO: 102 - DNA, 36 bp, Artificial
  oligonucleotide
gctatgaatagtcataagatttggatgttgccgcat

SEQ ID NO: 103 - DNA, 36 bp, Artificial
  oligonucleotide
ggtctgaagatttggagtttgccgccgcatcatggg

SEQ ID NO: 104 - DNA, 36 bp, Artificial
  oligonucleotide
aaggtttggtagatggcgcctacgactgcgttttcg

SEQ ID NO: 105 - DNA, 45 bp, Artificial
  oligonucleotide
acncayggnttyggncaycgngtntggtcngtnccnctncgntcn

SEQ ID NO: 106 - DNA, 45 bp, Artificial
  oligonucleotide
tcncgngtntcnggngcnaargtntggttyctntcnaaytggtcn

SEQ ID NO: 107 - DNA, 36 bp, Artificial
  oligonucleotide
gcnatgaaytcncayaarathtggatgctnccncay

SEQ ID NO: 108 - DNA, 36 bp, Artificial
  oligonucleotide
ggnctnaarathtggtcnctnccnccncaycayggn

SEQ ID NO: 109 - DNA, 36 bp, Artificial
  oligonucleotide
aargtntggcaratggcnccnacnacngcnttytcn

SEQ ID NO: 110 - protein, 330 aa, Homo sapiens
MEPNGTFSNNNSRNCTIENFKREFFPIVYLIIFFWGVLGNGLSIYVFLQPYKKSTSVNVFMLNLAISDLLFISTLPFRADYYLRGSNWIFGDLACRIMSYSLYVNMYSSIYFLTVLSVVRFLAMVHPFRLLHVTSIRSAWILCGIIWILIMASSIMLLDSGSEQNGSVTSCLELNLYKIAKLQTMNYIALVVGCLLPFFTLSICYLLIIRVLLKVEVPESGLRVSHRKALTTIIITLIIFFLCFLPYHTLRTVHLTTWKVGLCKDRLHKALVITLALAAANACFNPLLYYFAGENFKDRLKSALRKGHPQKAKTKCVFPVSVWLRKETRV

SEQ ID NO: 111 - protein, 337 aa, Homo sapiens
MNEPLDYLANASDFPDYAAAFGNCTDENIPLKMHYLPVIYGIIFLVGFPGNAVVISTYIFKMRPWKSSTIIMLNLACTDLLYLTSLPFLIHYYASGENWIFGDFMCKFIRFSFHFNLYSSILFLTCFSIFRYCVIIHPMSCFSIHKTRCAVVACAVVWIISLVAVIPMTFLITSTNRTNRSACLDLTSSDELNTIKWYNLILTATTFCLPLVIVTLCYTTIIHTLTHGLQTDSCLKQKARRLTILLLLAFYVCFLPFHILRVIRIESRLLSISCSIENQIHEAYIVSRPLAALNTFGNLLLYVVVSDNFQQAVCSTVRCKVSGNLEQAKKISYSNNP

SEQ ID NO: 112 - protein, 339 aa, Homo sapiens
MANLDKYTETFKMGSNSTSTAEIYCNVTNVKFQYSLYATTYILIFIPGLLANSAALWVLCRFISKKNKAIIFMINLSVADLAHVLSLPLRIYYYISHHWPFQRALCLLCFYLKYLNMYASICFLTCISLQRCFFLLKPFRARDWKRRYDVGISAAIWIVVGTACLPFPILRSTDLNNNKSCFADLGYKQMNAVALVGMITVAELAGFVIPVIIIAWCTWKTTISLRQPPMAFQGISERQKALRMVFMCAAVFFICFTPYHINFIFYTMVKETIISSCPVVRIALYFHPFCLCLASLCCLLDPILYYFMASEFRDQLSRHGSSVTRSRLMSKESGSSMIG

(19) United States
(12) Patent Application Publication
(10) Pub. No.: US 2004/0209808 A1
(43) Pub. Date: Oct. 21, 2004

(54) Modulators of human G-protein coupled receptors

(76) Inventor: Michael G. Kornacker, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/775,965

(22) Filed: Feb. 10, 2004

Related U.S. Application Data
(60) Provisional application No. 60/446,655, filed on Feb. 11, 2003.

(51) Int. Cl. A61K 38/17; C07K 14/705; C07H 21/04

(52) U.S. Cl. 514/12; 530/350; 435/69.1; 435/320.1; 435/325; 536/23.5

(57) ABSTRACT

The present invention relates to isolated peptides, and fragments and variants thereof, that bind to human G-protein coupled receptors, particularly G-protein coupled receptors belonging to the P2Y family of receptors. Also related are polypeptides comprising these peptides, and antibodies that specifically bind to these peptides and polypeptides. The invention further relates to polynucleotides encoding these peptides and polypeptides, vectors comprising these polynucleotides, and host cells comprising these vectors. In addition, the invention relates to methods for producing and isolating the disclosed peptides, polypeptides, antibodies, polynucleotides, vectors, and host cells. The invention also relates to methods for preventing, diagnosing, ameliorating, and treating disorders associated with HGPRBMY3 and related P2Y G-protein coupled receptors, including aberrant cell growth, immunological conditions, and diseases or disorders related to immune, brain, breast, cervix, kidney, and colon tissues. Further related are methods of screening for agents that modulate and/or bind to HGPRBMY3 or related P2Y G-protein coupled receptors.

This application claims benefit to provisional application U.S. Ser. No. 60/446,655 filed Feb. 11, 2003 under 35 U.S.C. 119(e). The entire teachings of the referenced application are incorporated herein by reference.

### FIELD OF THE INVENTION

The present invention relates to screening assays, diagnostics, and therapeutics. More specifically, the present invention relates to methods of diagnosing and/or treating diseases involving the Human G-Protein Coupled Receptor, HGPRBMY3, or related receptors, such as P2Y10, HGPRBMY11, and HGPRBMY23. Also related are methods of screening for agents that modulate and/or bind to HGPRBMY3 or other G-protein coupled receptors. In various aspects, the methods of the invention employ the disclosed receptor-binding peptides, polypeptides, and antibodies, as well as the polynucleotides, vectors, and host cells that can be used for production of these binders.

### BACKGROUND OF THE INVENTION

Many medically significant biological processes are mediated by the actions of G-proteins and/or second messengers (e.g., cAMP) in signal transduction pathways (Lefkowitz, Nature, 351:353-354 (1991)). Different G-proteins preferentially stimulate particular effectors to modulate specific biological functions in a cell. The targets for G-proteins include effector proteins, e.g., phospholipase C, adenylate cyclase, and phosphodiesterase, and actuator proteins, e.g., protein kinase A and protein kinase C (Simon, M. I., et al., Science, 252:802-8 (1991)). G-proteins, in turn, are targeted by G-protein coupled (GPC) receptors (also called GPCRs), including the receptors for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)).

In one type of signal transduction pathway, signaling is started when a hormone (e.g., dopamine, epineprhine, norepinephrine, or serotonin) binds to its cognate GPC receptor. The hormone-GPC receptor complex then binds to a G-protein. Upon binding of the complex, the G-protein converts from a GDP-bound, inactive state to a GTP-bound, active state. The G-protein then binds to and activates adenylate cyclase. Following this, the G-protein catalyzes hydrolysis of its bound GTP. The G-protein then returns to its GDP-bound, inactive state. In this type of pathway, the G-protein plays two roles, as an intermediate that relays the signal from the receptor to the effector, and as a clock that controls the duration of the signal. In addition, the G-protein effectively amplifies the signal from the bound hormone.

GPC receptors are a pharmacologically important protein family with more than 450 members identified to date. This family includes a wide range of biologically active receptors, such as neuroreceptors and hormone, viral, and growth factor receptors. The GPC receptor family specifically includes dopamine receptors, which bind to neuroleptic drugs, used for treating psychotic and neurological disorders. Other members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1, rhodopsins, odorant, and cytomegalovirus receptors, etc. Pathways involving GPC receptors are the targets of hundreds of drugs, including antihistamines, neuroleptics, antidepressants, and antihypertensives.

For GPC receptors, the amino-terminus is extracellular and the carboxy-terminus is intracellular. During signal transduction processes, the signal is received at the extracellular amino-terminus of the receptor. The signal may comprise an endogenous ligand, a chemical moiety, or light. This signal is then transduced through the membrane to the cytoplasmic side, where a heterotrimeric protein G-protein is activated and elicits a response (F. Horn et al., Recept. and Chann., 5: 305-314 (1998)). GPC receptors can be intracellularly coupled by heterotrimeric G-proteins to intracellular enzymes, ion channels, and transporters (see, Johnson et al., Endoc. Rev., 10:317-331(1989)). GPC receptors are found in numerous sites within a mammalian host.

GPC receptors contain seven transmembrane domains that are interconnected by extracellular or intracellular loops. The transmembrane domains contain conserved sequences of about 20 to 30 amino acids that form hydrophobic α-helixes. These domains are connected by at least eight hydrophilic loops containing divergent sequences. Most GPC receptors contain conserved cysteine residues in the first two extracellular loops. These cysteines form disulfide bonds that are believed to stabilize the GPC receptor structure. The seven transmembrane regions are designated as TM1, TM2, TM3, TM4, TM5, TM6, and TM7. TM3, in particular, has been implicated in GPC receptor-mediated signal transduction.

In GPC receptors, the ligand binding sites are hydrophilic pockets formed by the transmembrane domains. The hydrophilic sides of GPC receptor transmembrane helices are believed to face inward to create a polar ligand-binding sites. In several GPC receptors, the TM3 domain contains a ligand-binding site, and a aspartate residue that is critical for binding. Additionally, the TM5 domain (serine residue), TM6 domain (asparagine, phenylalanine, or tyrosine residue), and TM7 domain (phenylalanine or tyrosine) are involved in ligand binding. G-protein binding is controlled by phosphorylation and lipidation (palmitylation or farnesylation) of the GPC receptor. Phosphorylation of multiple serine residues in the third cytoplasmic loop and the carboxy-terminal tail of GPC receptors prevents interaction with G-proteins. For several GPC receptors (e.g., β-adrenergic receptor), phosphorylation by protein kinase A and/or specific receptor kinases causes receptor desensitization. Sensitivity is subsequently restored by removal of the attached phosphates by a phosphatase.

The malfunction of GPC receptors has been implicated in diseases such as Alzheimer's, Parkinson, diabetes, dwarfism, color blindness, retinal pigmentosa, and asthma. GPC receptors are also implicated in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). The receptors have also been shown to play a role in HIV infection (Y. Feng et al., Science, 272: 872-877 (1996)). Therefore, binders and modulators (e.g., ligands, agonists, antagonists, inhibitors, or activators) of GPC receptors are useful for both therapeutic and diagnostic applications.

### SUMMARY OF THE INVENTION

HGPRBMY3 has recently been identified as a human GPC receptor that may be involved in cellular growth processes in immune-, testes-, colon-, breast-, and ovarian-related tissues based on its abundance in these tissues (U.S. Ser. No. 10/268,332, filed Oct. 10, 2002; which is a continuation-in-part of U.S. Ser. No. 09/964,821, filed Sep. 26, 2001, which claims benefit to provisional application U.S. Ser. No. 60/235,713, filed Sep. 27, 2000; provisional application U.S. Ser. No. 60/261,783, filed Jan. 16, 2001; provisional application U.S. Ser. No. 60/305,085, filed Jul. 13, 2001; and provisional application U.S. Ser. No. 60/313,171, filed Aug. 17, 2001, which are hereby incorporated by reference in their entirety). HGPRBMY3 is expressed in a variety of human cancer cell lines, particularly leukemia, cervical cancer, melanoma, and ovarian cancer cells. In addition, HGPRBMY3 is over expressed in kidney, breast, and ovarian cancer tissues.

The present invention encompasses isolated peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof) that bind to HGPRBMY3 (e.g., SEQ ID NO:2), and/or a related GPC receptor (e.g., SEQ ID NO:110-SEQ ID NO:112), such as P2Y10, HGPRBMY11 (U.S. Ser. No. 09/991,225, filed Nov. 16, 2001, which is hereby incorporated by reference in its entirety), and HGPRBMY23 (U.S. Ser. No. 10/010,568, filed Dec. 7, 2001, which is hereby incorporated by reference in its entirety). In addition, the invention encompasses polypeptides comprising these peptides.

The invention also encompasses isolated nucleic acids that encode these peptides, polypeptides comprising these peptides, and fragments, variants, and derivatives thereof. Specific examples of isolated nucleic acids include those set forth in SEQ ID NO:47-SEQ ID NO:63, SEQ ID NO:67-SEQ ID NO:71, and SEQ ID NO:100-SEQ ID NO:104, and fragments, variants, and derivative thereof. Also encompassed are vectors that comprise the isolated nucleic acids, and host cells that comprise these vectors.

The invention further encompasses polypeptide complexes comprising HGPRBMY3 (e.g., SEQ ID NO:2) or a related GPC receptor (e.g., SEQ ID NO:110-SEQ ID NO:112) and one or more peptides (e.g., SEQ ID NO:17-SEQ ID NO:26, SEQ ID NO:35-SEQ ID NO:46, and SEQ ID NO:73-SEQ ID NO:77, or fragments, variants, or derivatives thereof) that bind to these receptors. Also encompassed are fragments of these polypeptide complexes.

The invention additionally encompasses antibodies and antibody fragments that specifically bind to the isolated peptides (e.g., SEQ ID NO:17-SEQ ID NO:26, SEQ ID NO:35-SEQ ID NO:46, and SEQ ID NO:73-SEQ ID NO:77), polypeptides or polypeptide complexes comprising these peptides, or fragments variants, or derivatives thereof. Further encompassed are anti-idiotype antibodies that specifically bind to an HGPRBMY3 receptor, or a related GPC receptor.

The present invention encompasses methods of producing the isolated peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), polypeptides comprising these peptides, and the corresponding nucleic acids (e.g., SEQ ID NO:47-SEQ ID NO:63, SEQ ID NO:67-SEQ ID NO:71, and SEQ ID NO:100-SEQ ID NO:104, and fragments, variants, and derivatives thereof). Also encompassed are methods of producing the vectors, host cells, and antibodies of the invention.

In specific aspects, the peptides of the invention (e.g., SEQ ID NO:17-SEQ ID NO:26, SEQ ID NO:35-SEQ ID NO:46, and SEQ ID NO:73-SEQ ID NO:77, and fragments, variants, and derivatives thereof) may be used to detect and/or measure levels of HGPRBMY3 (e.g., SEQ ID NO:2), or a related GPC receptor (e.g., SEQ ID NO:110-SEQ ID NO:112). In addition, certain peptides may be used to detect specific mutation(s) or truncation(s) of HGPRBMY3 or other GPC receptors. The invention therefore encompasses methods for detecting and/or measuring levels of GPC receptors, including HGPRBMY3, using the isolated peptides or polypeptides. Also encompassed are methods of using the isolated peptides or polypeptides of the invention to diagnose or monitor medical conditions (e.g., cancers, neoplasms, and other proliferative disorders) associated with altered levels or activity of HGPRBMY3 or a related GPC receptor; or alteration(s) or deletion(s) of the HGPRBMY3 sequence or a related receptor sequence. The anti-idiotype antibodies of the invention can be used for similar diagnostic and monitoring applications.

In other aspects, the peptides of the invention (e.g., SEQ ID NO:17-SEQ ID NO:26, SEQ ID NO:35-SEQ ID NO:46, and SEQ ID NO:73-SEQ ID NO:77, and fragments, variants, and derivatives thereof), or polypeptides comprising these peptides, may be used to modulate the activity of HGPRBMY3 (e.g., SEQ ID NO:2) or another GPC receptor (e.g., SEQ ID NO:110-SEQ ID NO:112). For example, the isolated peptides may act as agonists, antagonists, inhibitors (competitive or non-competitive), or surrogate ligands of one or more GPC receptors. In addition, certain peptides or combinations of these peptides may be formulated into pharmaceutical compositions.

The invention therefore encompasses methods of using the isolated peptides or polypeptides to prevent, ameliorate, or treat conditions (e.g., cancers, neoplasms, and other proliferative disorders) associated with altered expression or activity of HGPRBMY3 or a related GPC receptor. The nucleic acids, vectors, anti-idiotype antibodies, and host cells of the invention can be used for similar preventative and therapeutic applications.

The invention also encompasses methods of using the isolated peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof) or polypeptides for screening and identification other agents that bind to HGPRBMY3 or a related GPC receptor. The invention further encompasses methods using the isolated peptides (e.g., SEQ ID NO:17-SEQ ID NO:26, SEQ ID NO:35-SEQ ID NO:46, and SEQ ID NO:73-SEQ ID NO:77, or fragments, variants, or derivatives thereof) or polypeptides for screening and identification of other agents that modulate the levels or activity of a GPC receptor, such as HGPRBMY3 (e.g., agonists, antagonists, competitive or non-competitive inhibitors, or surrogate ligands). The anti-idiotype antibodies of the invention can be used in similar screens to identify modulating and/or binding agents of GPC receptors.

Additional aspects and advantages afforded by this invention will be apparent from the detailed description and exemplification herein below.

### BRIEF DESCRIPTION OF THE FIGURES

FIG. 1 shows the full-length nucleotide sequence (SEQ ID NO:1) of the cDNA clone containing HGPRBMY3, a human GPC receptor.

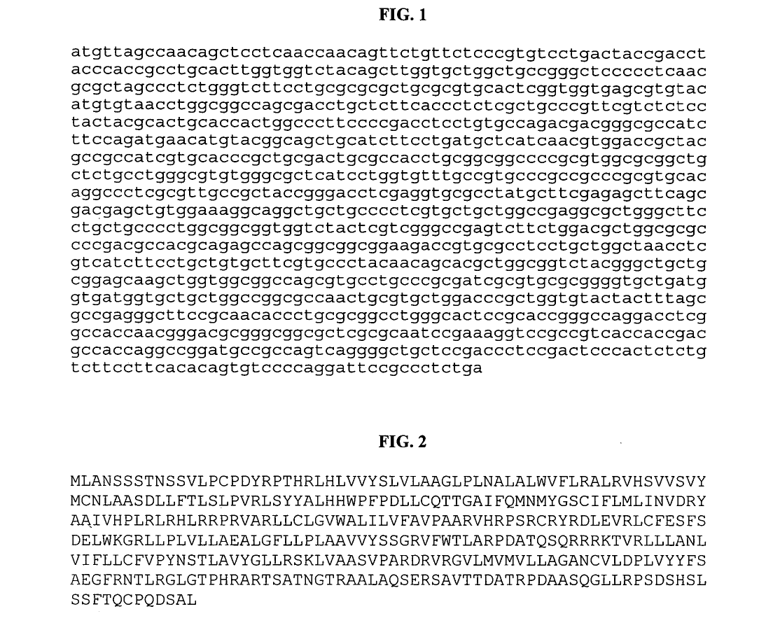

FIG. 2 shows the amino acid sequence (SEQ ID NO:2) from the conceptual translation of the full-length HGPRBMY3 cDNA sequence.

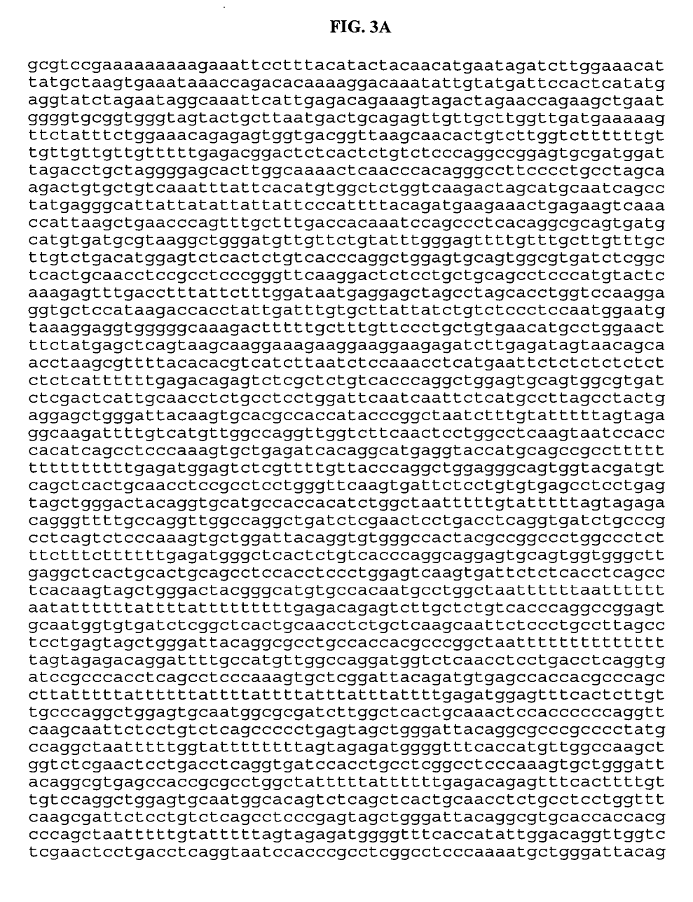

FIGS. 3A-3B show the 5′ untranslated sequence (SEQ ID NO:3) of the HGPRBMY3 gene.

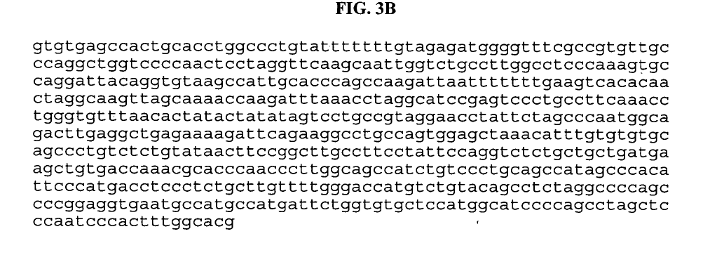

FIG. 4 shows the 3′ untranslated sequence (SEQ ID NO:4) of the HGPRBMY3 gene.

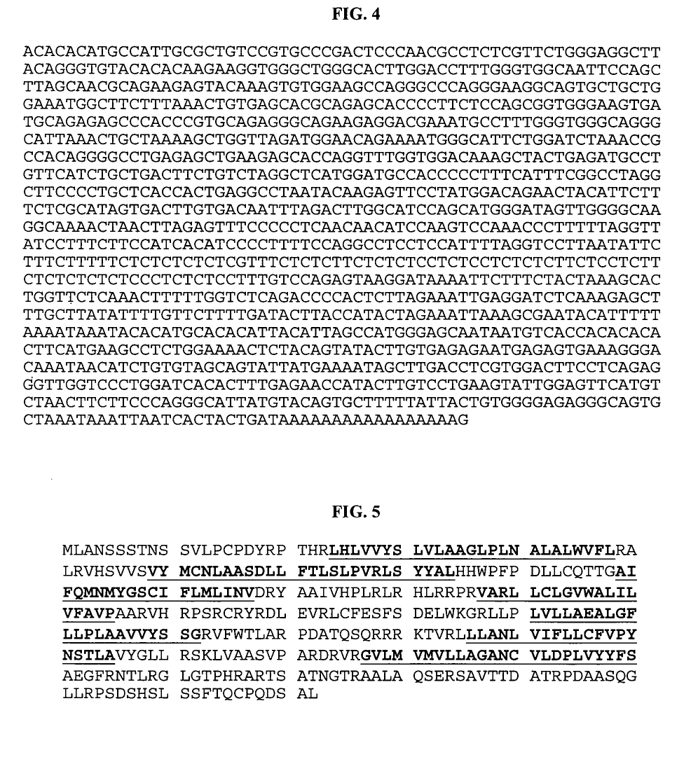

FIG. 5 shows the predicted transmembrane regions of the HGPRBMY3 polypeptide (SEQ ID NO:2). Predicted transmembrane domains corresponding to peaks with scores above 1000 are shown with bold text and underlining.

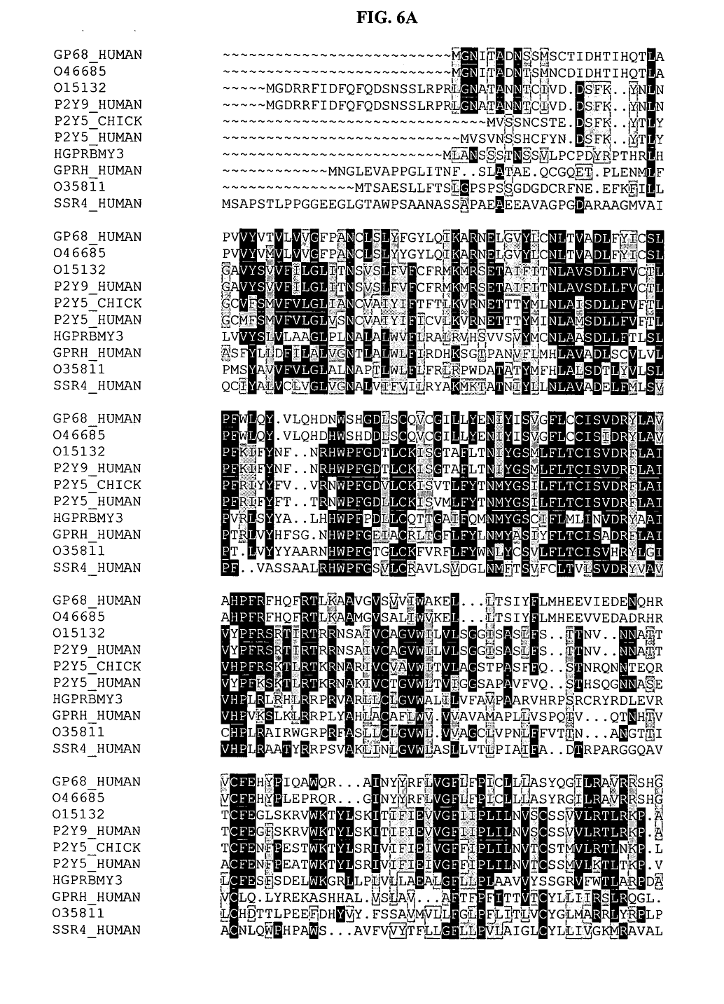

FIGS. 6A-6B show a multiple sequence alignment that includes HGPRBMY3 and other known GPC receptors (purinergic and somatostatin receptors), as described in Example 1. The black shading represents identical residues observed in more than half of the listed sequences. The gray shading represents similar residues observed in the listed sequences.

FIG. 7 shows the expression profiling of HGPRBMY3, as described in Example 3.

FIG. 8 shows the expression profiling of HGPRBMY3, as described in Table 1 and Example 4.

FIG. 9 shows the FACS profile for the untransfected CHO-NFAT/CRE cell line.

FIG. 10 shows the FACS profile for the CHO-NFAT/CRE cell line transfected with a vector overexpressing HGPRBMY3.

FIG. 11 shows the FACS profile for the untransfected CHO-NFAT cell line.

FIG. 12 shows the FACS profile for the CHO-NFAT/Gα15 cell line transfected with a vector overexpressing HGPRBMY3.

FIGS. 13A-13D show the results of indirect immunofluorescence analysis of FLAG®-tagged HGPRBMY3. FIG. 13A: CHO-NFAT/Gα15 control cells (fluorescent signal); FIG. 13B: CHO-NFAT/Gα15 control cells (bright field); FIG. 13C: CHO-NFAT/Gα15 cells transfected with HGPRBMY3-FLAG® (fluorescent signal); FIG. 13D: CHO-NFAT/Gα15 cells transfected with HGPRBMY3-FLAG® (bright field).

FIGS. 14A-14D show CHO-NFAT/CRE cell lines exhibiting intermediate and high beta-lactamase expression levels, which can be used in screens to identify HGPRBMY3 agonists and/or antagonists. FIG. 14A: CHO-NFAT/CRE cells (uninduced; −P/T/F); FIG. 14B: CHO-NFAT/CRE cells (induced; +P/T/F); FIG. 14C: CHO-NFAT/CRE cells transfected with an orphan GPC receptor (intermediate expression of beta-lactamase); FIG. 14D: CHO-NFAT/CRE cells transfected with an orphan GPC receptor (high expression of beta-lactamase).

FIG. 15 shows an expanded expression profile of HGPRBMY3 in various tissue sources, as described in Example 6.

FIG. 16 shows an expanded expression profile of HGPRBMY3 in various normal and tumor tissues, as described in Example 6.

FIG. 17 shows an expanded expression profile of HGPRBMY3 in various cancer cell lines, as described in Example 7. A key is provided in Table 2, below.

FIGS. 18A-18C show the nucleotide and amino acid sequence information for the HGPRBMY3-binding peptides, as described in Example 8. FIG. 18A: Amino acid sequence alignments of HGPRBMY3 binders illustrating shared binding motifs. Identical amino acid residues are shown in bold; similar amino acid residues are underlined. FIGS. 18B-18C: HGPRBMY3 binders and their coding sequences (specific and universal). Coding sequence key: B=A, C, or T; X=G, A, C, or T; O=G or A; Z=C or T.

FIGS. 19A-19B show the nucleotide and amino acid sequence information for peptides that bind to GPC receptors HGPRBMY11, HGPRBMY23, and P2Y10. FIG. 19A: Amino acid sequences of GPCR binders. Triplet sequences RVW, KVW, and KIW are shown by underlining. FIG. 19B: GPCR binders and their coding sequences (specific and universal). Coding sequence key: B=A, C, or T; X=G, A, C, or T; O=G or A; Z=C or T.

FIG. 20 shows the amino acid sequence information for HGPRBMY11 (SEQ ID NO:110), HGPRBMY23 (SEQ ID NO:111), and P2Y10 (GenBank Accession No. AF000545; SEQ ID NO:112).

### DETAILED DESCRIPTION OF THE PRESENT INVENTION

The present invention relates to isolated peptides (e.g., SEQ ID NO:17-SEQ ID NO:26, and SEQ ID NO:35-SEQ ID NO:46, and fragments, variants, and derivatives thereof) that bind to a novel human GPC receptor named HGPRBMY3. The invention also relates to isolated peptides (e.g., SEQ ID NO:73-SEQ ID NO:77, and fragments, variants, and derivatives thereof) that bind to one or more GPC receptors related to HGPRBMY3, such as P2Y10, HGPRBMY11, and HGPRBMY23. HGPRBMY3 shows high expression levels in immune-, testes-, colon-, and breast-related tissues, and moderately high expression levels in ovarian tissues. In addition, HGPRBMY3 is expressed in a variety of cancer cell lines, particularly leukemia, cervical cancer, melanoma, and ovarian cancer cell lines. HGPRBMY3 is also specifically overexpressed in kidney, breast, and ovarian cancer tissues. The expression pattern of HGPRBMY3 suggests that it is involved, directly or indirectly, in the modulation of cellular proliferation, and/or may represent a biomarker for transformed phenotypes. HGPRBMY3 may be a particularly important biomarker for leukemia, cervical cancer, melanoma, ovarian cancer, kidney cancer, and breast cancer.

The peptides of the present invention are useful for a variety of purposes, though most notably for modulating and/or binding to HGPRBMY3 (e.g., SEQ ID NO:2) or related GPC receptors (e.g., SEQ ID NO:110-SEQ ID NO:112). As modulators, the peptides may act as agonists, antagonists, inhibitors (competitive or non-competitive), or surrogate ligands of HGPRBMY3. The peptides may also modulate and/or bind to other GPC receptors of the same subclass (e.g., peptide receptors, adrenergic receptors, purinergic receptors, etc.), and/or other subclasses known in the art. As specific examples, the peptides of the present invention may be useful as agonists of HGPRBMY3 or related GPC receptors. Alternatively, the peptides may be useful as antagonists of HGPRBMY3 or related GPC receptors. In addition, the peptides of the present invention may be useful as competitive inhibitors of the cognate ligand(s) of the GPC receptors, or may be useful as non-competitive inhibitors of the cognate ligand(s). Furthermore, the peptides of the present invention may be used in assays designed to identify the ligand(s) of HGPRBMY3 or related GPC receptors, or to identify other modulatory agents, e.g., agonists or antagonists of the receptors, particularly small molecule modulators. The modulatory effect of the peptides or other agents may be determined by adding an effective amount of each peptide to each functional assay. Representative functional assays are described more specifically herein.

### Definitions

Use of the phrase “SEQ ID NO:17-SEQ ID NO:26, SEQ ID NO:35-SEQ ID NO:46, and SEQ ID NO:73-SEQ ID NO:77” etc., is intended, for convenience, to refer to each individual SEQ ID NO. individually, and is not intended to refer to the sequences collectively. The invention encompasses each sequence individually, as well as any combination thereof.

The term “binders,” e.g., “GPCR binders,” refers to peptides, polypeptides, antibodies, etc. of the invention which bind to HGPRBMY3 or one or more related GPC receptors, such as P2Y10, HGPRBMY11, and HGPRBMY23. The invention encompasses each binder individually, as well as any combination thereof.

The HGPRBMY3, HGPRBMY11, HGPRBMY23, and P2Y10 polypeptide (or protein) refers to the amino acid sequence of substantially purified polypeptide (e.g., SEQ ID NO:2 or SEQ ID NO:110-SEQ ID NO:112), which may be obtained from any species, preferably mammalian, and more preferably, human, and from a variety of sources, including cellular, cell-free, synthetic, semi-synthetic, or recombinant sources. Functional equivalents of a polypeptide (e.g., fragments, variants, and derivatives) are also embraced by the present invention. HGPRBMY3 is also referred to herein as GPCR19 and GPR92.

The disclosed peptides include amino acid sequences (e.g., SEQ ID NO:17-SEQ ID NO:26, SEQ ID NO:35-SEQ ID NO:46, and SEQ ID NO:73-SEQ ID NO:77, and fragments, variants, and derivatives) that bind to HGPRBMY3, a related GPC receptor, or functional equivalents thereof. The peptides may be obtained from synthetic, semi-synthetic, cell-free, or recombinant sources.

The term “agonist” refers to a molecule which, when bound to HGPRBMY3, or a related GPC receptor, or a functional fragment thereof, increases or prolongs the activity of the polypeptide. Agonists may include proteins, nucleic acids, carbohydrates, compounds, materials, agents, drugs, and any other molecules that inhibit, reduce, block, suppress, diminish, decrease, or eliminate GPC receptor activity. The term “antagonist” refers to a molecule which, when bound to HGPRBMY3, or a related GPC receptor, or a functional fragment thereof, decreases the amount or duration of the activity of the polypeptide. Antagonists may include proteins, nucleic acids, carbohydrates, antibodies, compounds, materials, agents, drugs, and any other molecules that agonize, enhance, increase, augment, or amplify HGPRBMY3 activity.

As used herein the term “modulate” refers to an increase or decrease in the levels, stability, or activity of HGPRBMY3 or a related GPC receptor, or the mRNA or DNA coding sequences. The term “modulating agent” is meant to encompass enhancing agents, agonists, inverse agonists, antagonists (e.g., competitive reversible, irreversible, and negative antagonists), and inhibitors (e.g., competitive and non-competitive inhibitors) of HGPRBMY3 or another GPC receptor. The modulating agents of the invention may affect the GPC receptors directly, or may affect downstream components that are regulated by, or which interact with, the receptors in the cell. Modulating agents may include proteins, nucleic acids, carbohydrates, antibodies, compounds, materials, agents, drugs, and any other molecules.

The term “nucleic acid sequence,” as used herein, refers to a nucleotide, oligonucleotide, or polynucleotide sequence, and a fragment or portion thereof. Specifically included are DNA and RNA sequences obtained from cellular, cell-free, or synthetic sources. Further included are genomic and cDNA sequences, unspliced or partly spliced transcripts, and splicing products. Also included are “protein nucleic acids” (PNAs) formed by conjugating bases to an amino acid backbone. The nucleic acid sequences of the invention may be single- or double-stranded (i.e., DNA-DNA, DNA-RNA and RNA-RNA hybrids), and may represent the sense or antisense strand. By way of non-limiting example, nucleic acid fragments are at least 20-60 contiguous nucleotides, preferably at least 70-100 contiguous nucleotides, or at least 1000 contiguous nucleotides or greater in length.

“Variant” nucleic acid sequences include nucleic acid sequences containing one or more deletions, insertions and/or substitutions of different nucleotides resulting in a polynucleotide that encodes the same or a functionally-equivalent GPC receptor or binder. Functional equivalents, preferably, retain at least one activity (e.g., coupling, signaling, binding, modulatory, or antigenic activity) of the original polypeptide or peptide.

“Derivative” nucleic acid sequences include nucleic acid sequences subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acid sequences may also contain labels, including radionucleotides, enzymes, fluorescent agents, chemiluminescent agents, chromogenic agents, substrates, cofactors, inhibitors, magnetic particles, and the like. Preferably, a nucleic acid derivative encodes a functional equivalent of HGPRBMY3, or a related GPC receptor, or binder, which retains at least one activity (e.g., coupling, signaling, binding, modulatory, or antigenic activity) of the original polypeptide or peptide.

“Oligonucleotides” or “oligomers” refer to a nucleic acid sequence, preferably comprising contiguous nucleotides, of at least about 6 nucleotides to about 60 nucleotides, preferably at least about 8 to 10 nucleotides in length, more preferably at least about 12 nucleotides in length e.g., about 15 to 35 nucleotides, or about 15 to 25 nucleotides, or about 20 to 35 nucleotides, which can be typically used in PCR amplification assays, hybridization assays, or in microarrays. It will be understood that the term “oligonucleotide” is substantially equivalent to the terms primer, probe, or amplimer, as commonly defined in the art. It will also be appreciated by those skilled in the pertinent art that a longer oligonucleotide probe, or mixtures of probes, e.g., degenerate probes, can be used to detect longer, or more complex, nucleic acid sequences, for example, genomic DNA. In such cases, the probe may comprise at least 20-200 nucleotides, preferably, at least 30-100 nucleotides, more preferably, 50-100 nucleotides.

“Amplification” refers to the production of additional copies of a nucleic acid sequence and is generally carried out using polymerase chain reaction (PCR) technologies, which are well known and practiced in the art (see, D. W. Dieffenbach and G. S. Dveksler, 1995, PCR Primer, a Laboratory Manual, Cold Spring Harbor Press, Plainview, N.Y.).

“Microarray” is an array of distinct polynucleotides or oligonucleotides synthesized on a substrate, such as paper, nylon, or other type of membrane; filter; chip; glass slide; or any other type of suitable solid support.

The term “consensus” refers to the sequence that reflects the most common choice of base or amino acid at each position among a series of related DNA, RNA or protein sequences. Areas of particularly good agreement often represent conserved functional domains.

The phrase “amino acid sequence,” as used herein, refers to an oligopeptide, peptide, polypeptide, and protein sequence, and a fragment or portion thereof. Amino acid sequences include naturally occurring, recombinant, and synthetic molecules. By way of non-limiting example, amino acid sequence fragments are at least 5-30, preferably 5-15 amino acids in length, and retain at least one activity (e.g., coupling, signaling, binding, or antigenic activity) of the full-length GPC receptor (e.g., SEQ ID NO:2 or SEQ ID NO:110-SEQ ID NO:112) or binder. The recitation of the terms “amino acid sequence,” “polypeptide,” and “protein” is not meant to limit the amino acid sequence to the full-length amino acid sequence associated with the recited protein or peptide molecule. The terms “polypeptide” and “HGPRBMY3 protein” are used interchangeably herein to refer to the encoded product of the HGPRBMY3 nucleic acid sequence of the present invention.

A “variant” of a GPC receptor or a GPCR-binding peptide refers to an amino acid sequence that contains one or more deletions, insertions and/or substitutions of different residues. The variant may have “conservative” changes, wherein a substituted amino acid has similar structural or chemical properties, e.g., replacement of leucine with isoleucine. More rarely, a variant may have “non-conservative” changes, e.g., replacement of a glycine with a tryptophan. Minor variations may also include amino acid deletions or insertions, or both. Deliberate amino acid substitutions may be made on the basis of similarity in polarity, charge, solubility, hydrophobicity, hydrophilicity, and/or the amphipathic nature of the residues, as long as at least one biological activity (e.g., coupling, signaling, binding, modulatory, or antigenic activity) of a GPC receptor or GPCR-binding peptide is retained.

As non-limiting examples, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine. Guidance in determining which amino acid residues may be substituted, inserted, or deleted without abolishing functional biological or immunological activity may be found using computer programs well known in the art, for example, DNASTAR software, Vector NTI, and, specifically, the application of the PAM250 or BLOSUM62 matrix.

A “derivative” amino acid sequence is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label. Preferably, the derivative amino acid sequence retains at least one biological activity (e.g., coupling, signaling, binding, modulatory, or antigenic activity) of the original sequence.

The term “biologically active”, i.e., functional, refers to a polypeptide, peptide or fragment thereof having structural, regulatory, antigenic, or biochemical activities of the original molecule.

The term “substantially purified” refers to nucleic acid sequences or amino acid sequences that are isolated or separated, and are at least 60% free, preferably 75% to 85% free, and most preferably 90% or greater free from other components with which they are associated with in a cell, cell extract, or cell-free system.

The term “sample” is meant to be interpreted in its broadest sense. A “sample” refers to a biological sample, such as, for example, cells, tissues, or fluids (including, without limitation, plasma, serum, cerebrospinal fluid, lymph, tears, saliva, milk, pus, and tissue exudates and secretions) isolated from an individual or from in vitro cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “mimetic” refers to a molecule, the structure of which is developed from knowledge of the structure of a GPC receptor or GPCR-binding peptide, or portions thereof, and as such, is able to effect some or all of the actions of the polypeptide or peptide.

The term “portion” with regard to a polypeptide or peptide refers to fragments or segments of that amino acid sequence. The fragments may range in size from four or five amino acid residues to the entire amino acid sequence minus one amino acid. Thus, a peptide “comprising at least a portion of the amino acid sequence of SEQ ID NO:17-SEQ ID NO:26, SEQ ID NO:35-SEQ ID NO:46, or SEQ ID NO:73-SEQ ID NO:77” encompasses the full-length peptide, and fragments thereof.

The term “antibody” refers to intact molecules as well as fragments thereof, such as Fab, F(ab′)₂, Fv, which are capable of binding an epitopic or antigenic determinant. Antibodies that bind to GPC receptors or GPCR-binding peptides can be prepared using intact polypeptides or peptides, or fragments containing sequences of interest or prepared recombinantly for use as the immunizing antigen. The polypeptide, oligopeptide, or peptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g., a mouse, a rat, or a rabbit).

The term “humanized” antibody refers to antibody molecules in which amino acids have been replaced in the non-antigen binding regions in order to more closely resemble a human antibody, while still retaining the original binding capability, e.g., as described in U.S. Pat. No. 5,585,089 to C. L. Queen et al.

The term “antigenic determinant” refers to that portion of a molecule that makes contact with a particular antibody (i.e., an epitope). When a polypeptide, peptide, or fragment thereof is used to immunize a host animal, numerous regions of the sequence may induce the production of antibodies which bind specifically to a given region or three-dimensional structure on the sequence; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a polypeptide or peptide and a binding molecule, such as an agonist, an antagonist, or an antibody. The interaction is dependent upon the presence of a particular structure (i.e., an antigenic determinant or epitope) of the protein that is recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such methodologies include the following: Rees, S., Brown, S., Stables, 1999, J. “Reporter gene systems for the study of G Protein Coupled Receptor signaling in mammalian cells” In Milligan G. (ed.), Signal Transduction: A practical approach. Oxford: Oxford University Press, 171-221; Alam, J., Cook, J. L., 1990, “Reporter Genes: Application to the study of mammalian gene transcription”. Anal. Biochem. 188:245-254; Selbie, L. A. and Hill, S. J., 1998, “G protein-coupled receptor cross-talk: The fine-tuning of multiple receptor-signaling pathways” TiPs 19:87-93; and Boss, V., Talpade, D. J., and Murphy, T. J., 1996, “Induction of NFAT mediated transcription by Gq-coupled Receptors in lympoid and non-lymphoid cells” JBC 271:10429-10432.

Additional publications include: George, S. E., Bungay, B. J., and Naylor, L. H., 1997, “Functional coupling of endogenous serotonin (5-HT1B) and calcitonin (C1a) receptors in CHO cells to a cyclic AMP-responsive luciferase reporter gene” J. Neurochem. 69:1278-1285; Suto, C M, Igna D M, 1997, “Selection of an optimal reporter for cell-based high-throughput screening assays” J. Biomol. Screening 2:7-12; Zlokarnik, G., Negulescu, P. A., Knapp, T. E., More, L., Burres, N., Feng, L., Whitney, M., Roemer, K., and Tsien, R. Y., 1998, “Quantitation of transcription and clonal selection of single living cells with a B-Lactamase Reporter” Science 279:84-88; S. Fiering et. al., 1990, Genes Dev. 4:1823; J. Kattunen and N. Shastri, 1991, PNAS 88:3972; Hawes, B. E., Luttrell. L. M., van Biesen, T., and Lefkowitz, R. J., 1996, JBC271:12133-12136; Gilman, A. G., 1987, Annul. Rev. Biochem. 56: 615-649; and Maniatis et al., 1989, Cold Spring Harbor Press.

Further publications include: Salcedo, R., Ponce, M. L., Young, H. A., Wasserman, K., Ward, J. M., Kleinman, H. K., Oppenheim, J. J., Murphy, W. J., 2000, “Human endothelial cells express CCF2 and respond to MCP-1: direct role of MCP-1 in angiogenesis and tumor progression” Blood 96(1):34-40; Sica, A., Saccani, A., Bottazzi, B., Bernasconi, S., Allavena, P., Gaetano, B., LaRossa, G., Scotton, C., Balkwill F., Mantovani, A, 2000, “Defective expression of the monocyte chemotactic protein 1 receptor CCF2 in macrophages associated with human ovarian carcinoma” J. Immunology 164:733-8; Kypson, A., Hendrickson, S., Akhter, S., Wilson, K., McDonald, P., Lilly, R., Dolber, P., Glower, D., Lefkowitz, R., Koch, W., 1999, “Adenovirus-mediated gene transfer of the B2 AR to donor hearts enhances cardiac function” Gene Therapy 6:1298-304; Dorn, G. W., Tepe, N. M., Lorenz, J. N., Kock, W. J., Ligget, S. B., 1999, “Low and high level transgenic expression of B2AR differentially affect cardiac hypertrophy and function in Gαq-overexpressing mice” PNAS 96:6400-5; J. Wess, 1997, “G protein coupled receptor: molecular mechanisms involved in receptor activation and selectivity of G-protein recognition” FASEB 11:346-354.

Also included are: Whitney, M, Rockenstein, E, Cantin, G., Knapp, T., Zlokarnik, G., Sanders, P., Durick, K., Craig, F. F., and Negulescu, P. A., 1998, “A genome-wide functional assay of signal transduction in living mammalian cells” Nature Biotech. 16:1329-1333; BD Biosciences, FACS Vantage SE Training Manual Part Number 11-11020-00 Rev. A. August, 1999; Chen, G., Jaywickreme, C., Way, J., Armour S., Queen K., Watson., C., Ignar, D., Chen, W. J., Kenakin, T., 1999, “Constitutive Receptor systems for drug discovery”. J. Pharmacol. Toxicol. Methods 42:199-206; Blahos, J., Fischer, T., Brabet, I., Stauffer, D., Rovelli, G., Bockaert, J., and Pin, J.-P., 2001, “A novel Site on the G alpha-protein that Recognized Heptahelical Receptors” J. Biol. Chem. 275(5):3262-69; Offermanns, S. & Simon, M. I., 1995, “G alpha 15 and G alpha 16 Couple a Wide Variety of Receptors to Phospholipase C”. J. Biol. Chem. 270(25):15175-80.

Information relating to G-protein coupled receptors may be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11:1-20(1992); Savarese T. M., Fraser C. M., Biochem. J. 283:1-9(1992); Branchek T., Curr. Biol. 3:315-317(1993); Stiles G. L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Garnier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); hypertext transfer protocol://world wide web.gcrdb.uthscsa.edu/; and hypertext transfer protocol://swift.embl-heidelberg.de/7tm/.

All of these publications are incorporated by reference herein in their entirety.

### Nucleic Acid Sequences

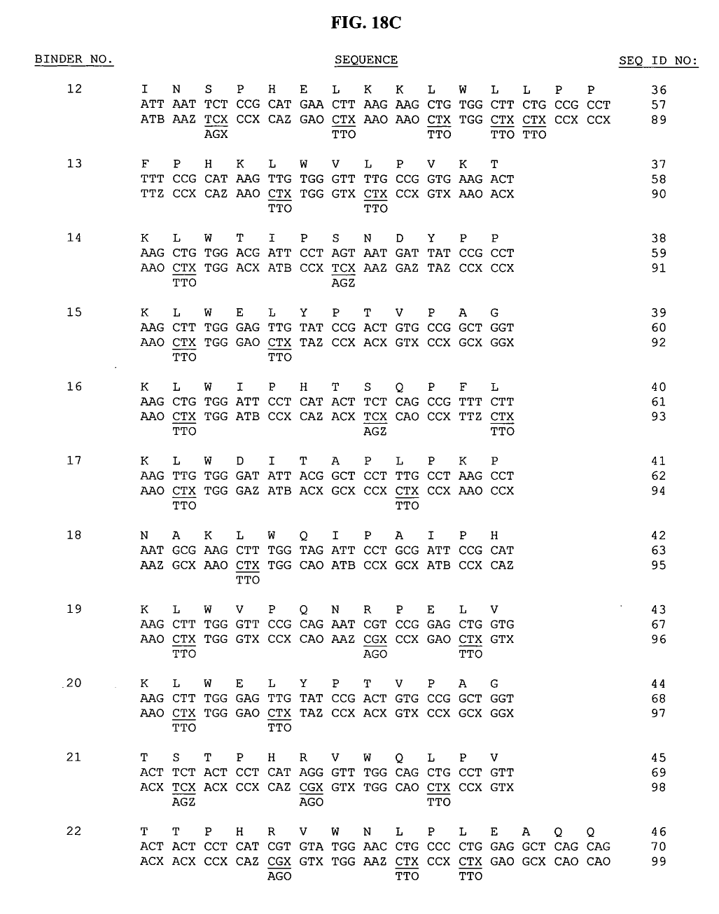

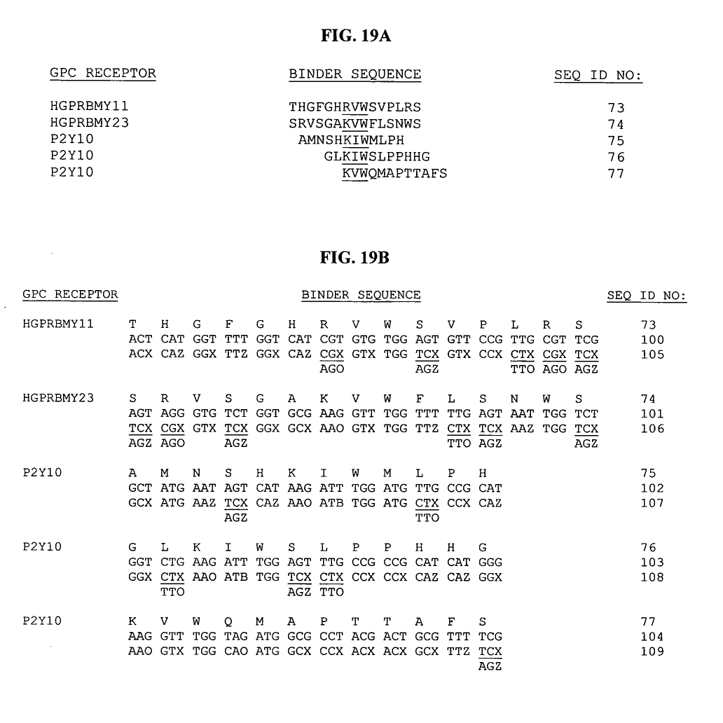

The present invention encompasses nucleic acid sequences that encode peptides (e.g., SEQ ID NO:17-SEQ ID NO:26, SEQ ID NO:35-SEQ ID NO:46, and SEQ ID NO:73-SEQ ID NO:77, or fragments, variants, or derivatives thereof) that bind to HGPRBMY3 or a related GPC receptor, such as HGPRBMY11, HGPRBMY23, and P2Y10. Any nucleic acid sequence that encodes an amino acid sequence of an HGPRBMY3-binding peptide, a polypeptide comprising this peptide, or related fragments or variants is included in the invention. In a specific aspect, the present invention encompasses nucleic acid sequences comprising one or more of SEQ ID NO:47-SEQ ID NO:63, SEQ ID NO:67-SEQ ID NO:71, and SEQ ID NO:100-SEQ ID NO:104, or fragments, variants, or derivatives thereof. Nucleic acid variants of the present invention include, but are not limited to, variants that share at least 40%, 50%, 60%, 70%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% nucleotide sequence identity with any one of SEQ ID NO:47-SEQ ID NO:63, SEQ ID NO:67-SEQ ID NO:71, and SEQ ID NO:100-SEQ ID NO:104. Preferred are variants that share at least 45%, 51%, or 56% identity with any one of SEQ ID NO:47-SEQ ID NO:63, SEQ ID NO:67-SEQ ID NO:71, and SEQ ID NO:100-SEQ ID NO:104. In another aspect, the invention encompasses nucleic acid sequence fragments, which include, but are not limited to, fragments comprising at least 8, 10, 12, 15, 18, 20, 25, 30, 35, 36, 40, 45 contiguous nucleotides of any one of SEQ ID NO:47-SEQ ID NO:63, SEQ ID NO:67-SEQ ID NO:71, and SEQ ID NO:100-SEQ ID NO:104. Preferably, fragments comprise at least 18, 19, 20, or 21 contiguous nucleotides of any one of SEQ ID NO:47-SEQ ID NO:63, SEQ ID NO:67-SEQ ID NO:71, and SEQ ID NO:100-SEQ ID NO:104. More preferably, the fragments encode one or more binding motifs, including KIW, KVW, KLW, RVW, and TPHRVWXLP (SEQ ID NO:27), wherein X is Q or N (see FIGS. 18A and 19A).

Also encompassed by the invention are nucleic acid sequences that are capable of hybridizing to the disclosed nucleotide sequences (e.g., SEQ ID NO:47-SEQ ID NO:63, SEQ ID NO:67-SEQ ID NO:71, and SEQ ID NO:100-SEQ ID NO:104) under various conditions of stringency. Hybridization conditions are typically based on the melting temperature (Tm) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to any one of SEQ ID NO:47-SEQ ID NO:63, SEQ ID NO:67-SEQ ID NO:71, and SEQ ID NO:100-SEQ ID NO:104, and other sequences that are degenerate to those which encode a GPC receptor-binding peptide. As a non-limiting example, moderate stringency conditions comprise a prewashing solution of 2×SSC, 0.5% SDS, 1.0 mM EDTA, pH 8.0, and hybridization conditions of 50° C., 5×SSC, and overnight incubation.

As will be appreciated by the skilled practitioner in the art, the degeneracy of the genetic code results in the production of a multitude of nucleotide sequences encoding a GPCR-binding peptide (e.g., SEQ ID NO:89-SEQ ID NO:99 and SEQ ID NO:105-SEQ ID NO:104). Specific and universal coding sequences are provided in FIGS. 18B, 18C, and 19B. For the universal coding sequences shown in these figures, alternative codons are indicated by underlining. As an example, for HGPRBMY3 binder no. 1, the leucine at position 1 is encoded by universal codons CTX or TTO, wherein X is G, A, T, or C, and O is G or A (FIG. 18B). Some of the degenerate sequences may bear minimal homology to the nucleotide sequences of the originally identified GPCR-binders (e.g., SEQ ID NO:47-SEQ ID NO:63, SEQ ID NO:67-SEQ ID NO:71, and SEQ ID NO:73-SEQ ID NO:77). Accordingly, the present invention contemplates each and every possible variation of nucleotide sequence that could be made by selecting combinations based on possible codon choices. These combinations are made in accordance with the standard triplet genetic code as applied to the nucleotide sequence of the originally identified GPCR binders, and all such variations are to be considered as being specifically disclosed.

For some purposes, it may be advantageous to produce nucleotide sequences encoding a GPCR-binding peptide, or its fragments, variants, or derivatives, which possess a substantially different codon usage. Codons may be selected to increase the rate at which expression of the peptide/polypeptide occurs in a particular prokaryotic or eukaryotic host in accordance with the frequency with which particular codons are utilized by the host. Alterations in codon usage can also be used to produce RNA transcripts having more desirable properties, such as a greater half-life, than transcripts produced from the naturally occurring sequence. In particular, RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

The nucleic acid sequences of the invention (e.g., SEQ ID NO:47-SEQ ID NO:63, SEQ ID NO:67-SEQ ID NO:71, and SEQ ID NO:100-SEQ ID NO:104, and fragments thereof) can be engineered using methods generally known in the art in order to alter the sequences for a variety of reasons, including, but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and the like.

Also encompassed by the invention derivatives of the nucleic acid sequences (e.g., SEQ ID NO:47-SEQ ID NO:63, SEQ ID NO:67-SEQ ID NO:71, and SEQ ID NO:100-SEQ ID NO:104, and fragments thereof), which comprise one or more chemical modification, for example, replacement of hydrogen by an alkyl, acyl, or amino group. Alternatively, derivative nucleic acid sequences may comprise non-naturally-occurring portions, such as altered sugar moieties or inter-sugar linkages. Exemplary among these are phosphorothioate and other sulfur containing species which are known in the art. Derivative nucleic acid sequences may also contain detection labels, including radionucleotides (e.g., ³²P, 3H, and ³⁵S), enzymes, fluorescent (e.g., rhodamine, fluorescein, and Cy™3, Cy™5), chemiluminescent, or chromogenic, and other labels (e.g., DNP, digoxigenin, and biotin) such as substrates, cofactors, inhibitors, magnetic particles, and the like.

A wide variety of labels and conjugation techniques are known and employed by those skilled in the art. Nucleic acid labeling can be achieved by oligo-labeling, nick translation, end-labeling, or PCR amplification using a labeled primer. Alternatively, nucleic acid sequences, or any portions or fragments thereof, may be cloned into a vector for the production of labeled mRNA sequences. Such vectors are known in the art, are commercially available, and may be used to synthesize labeled RNA in vitro by addition of an appropriate RNA polymerase, such as T7, T3, or SP(6) and labeled nucleotides. These procedures may be conducted using a variety of commercially available kits (e.g., from Amersham-Pharmacia; Promega Corp.; and U.S. Biochemical Corp., Cleveland, Ohio).

The present invention also encompasses the production of nucleic acid sequences, or portions thereof, which encode a GPCR-binding peptide, and its fragments, and derivatives, entirely by synthetic chemistry (see, for example, M. H. Caruthers et al., 1980, Nucl. Acids Res. Symp. Ser., 215-223 and T. Horn et al., 1980, Nucl. Acids Res. Symp. Ser., 225-232). After production, the synthetic sequence may be inserted into any of the many available expression vectors and cell systems using reagents that are well known to those in the art. Moreover, synthetic chemistry may be used to introduce mutations into a sequence encoding a GPCR-binding peptide, or any fragment, variant, or derivative thereof. Alternatively, the nucleic acid sequences of the invention can be produced by PCR amplification of the cloned sequences. In addition, the nucleic acid sequences may be produced by recombinant systems, including cell-based and cell-free systems.

Nucleic acid sequences which encode a GPCR-binding peptide, or fragments, variants, or derivatives thereof, may be used in recombinant DNA molecules to direct the expression of a GPCR binder, or fragments or functional equivalents thereof, in appropriate host cells. Because of the inherent degeneracy of the genetic code, other DNA sequences, which encode substantially the same or a functionally equivalent amino acid sequence, may be produced and these sequences may be used to clone and express a GPCR-binding peptide. For expression in recombinant systems, a start and stop codons may be added to the nucleic acid sequence of a GPCR-binding peptide. In addition, nucleotide sequences encoding epitopes or protein tags can be added to the nucleic acid sequence of the GPCR binder, as described in detail herein.

### Sequence Variants

Over the years, there have been a number of methods developed to introduce mutations into macromolecules. Some of these methods include, random mutagenesis, error-prone PCR, chemical mutagenesis, site-directed mutagenesis, and other methods well known in the art. For a comprehensive listing of current mutagenesis methods, see Maniatis, Molecular Cloning: A Laboratory Manual, Cold Spring Harbor Press, Cold Spring, N.Y. (1982). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of error-prone PCR (as described in Moore, J., et al., Nature Biotechnology 14:458, (1996), or through the application of randomized synthetic oligonucleotides corresponding to specific regions of interest (as described by Derbyshire, K. M. et al., Gene, 46:145-152, (1986), and Hill, D E, et al., Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of a protein are fairly rare. In fact, using too high a level of mutagenesis may counter or inhibit the desired benefit of a useful mutation.

While both of the aforementioned methods are effective for creating randomized pools of macromolecule variants, a third method, termed DNA shuffling (WPC, Stemmer, PNAS, 91:10747, (1994)), has recently been elucidated. DNA shuffling has also been referred to as directed molecular evolution, exon-shuffling, directed enzyme evolution, in vitro evolution, and artificial evolution. Such reference terms are known in the art and are encompassed by the invention. This new, preferred, method apparently overcomes the limitations of the previous methods in that it not only propagates positive traits, but also eliminates negative traits in the resulting progeny.

DNA shuffling accomplishes this task by combining the principal of in vitro recombination, along with the method of error-prone PCR. In effect, a randomly digested pool of small fragments of the gene of interest, created by Dnase I digestion, is introduced into an “error-prone” PCR assembly reaction. During the PCR reaction, the randomly sized DNA fragments not only hybridize to their cognate strand, but also may hybridize to other DNA fragments corresponding to different regions of the polynucleotide of interest, i.e., regions not typically accessible via hybridization of the entire polynucleotide. Moreover, since the PCR assembly reaction utilizes error-prone PCR reaction conditions, random mutations are introduced during the DNA synthesis step of the PCR reaction for all of the fragments. This acts to further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly, the DNA substrate is prepared for the reaction. Preparation may involve purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example. Once the DNA substrate has been purified, it is subjected to Dnase I digestion. About 2-4 μg of the DNA substrate(s) is digested with 0.0015 units of Dnase I (Sigma) per μl in 100 μl of 50 mM Tris-HCL, pH 7.4/1 mM MgCl₂. The reaction is continued for 10⁻²⁰ min. at room temperature. The resulting fragments of 10⁻⁵⁰ bp are then separated by electrophoresis on a 2% low-melting point agarose gel. This is followed by purification using DE81 ion-exchange paper (Whatman), Microcon concentrators (Amicon) of the appropriate molecular weight cut-off, oligonucleotide purification columns (Qiagen), or other methods known in the art. For DE81 ion-exchange paper, the 10⁻⁵⁰ bp fragments are eluted using 1 M NaCl, followed by ethanol precipitation.

The resulting purified fragments are then be subjected to a PCR assembly reaction by re-suspension in a PCR mixture containing 2 mM of each dNTP, 2.2 mM MgCl₂, 50 mM KCl, 10 mM Tris●HCL, pH 9.0, and 0.1% Triton X-100, at a final fragment concentration of 10⁻³⁰ ng/μl. No primers are added at this point. Taq DNA polymerase (Promega) is used at 2.5 units per 100 μl of reaction mixture. A PCR program includes incubation at 94 C for 60 sec; followed by incubation at 94 C for 30 sec, 50-55 C for 30 sec, 72 C for 30 sec for 30-45 cycles; followed by incubation at 72 C for 5 min. For the reaction, a MJ Research (Cambridge, Mass.) PTC-150 thermocycler is used.

After the assembly reaction is completed, a 1:40 dilution of the resulting primerless product is introduced into a PCR mixture, using the same buffer mixture used for the assembly reaction, which contains 0.8 μm of each primer. This mixture is subjected to 15 cycles of PCR, with incubation at 94 C for 30 sec, 50 C for 30 sec, and 72 C for 30 sec. The preferred primers are those corresponding to the nucleic acid sequences of the polynucleotide(s) utilized in the shuffling reaction. The primers may be synthesized to contain modified nucleic acid base pairs using methods known in the art and referred to else where herein, or to contain additional sequences (i.e., for adding restriction sites, mutating specific base-pairs, etc.). The resulting shuffled, assembled, and amplified product can be purified using methods well known in the art (e.g., Qiagen PCR purification kits) and then subsequently cloned using appropriate restriction enzymes. Although a number of variations of DNA shuffling have been published to date, such variations would be known to the skilled artisan and are encompassed by the invention. The DNA shuffling method can also be tailored to the desired level of mutagenesis using the methods described by Zhao, et al. (Nucl Acid Res., 25(6):1307-1308, (1997).

As described above, once the randomized pool has been created, it is subjected to a specific screen to identify the variant possessing the desired characteristic(s). Once the variant is identified, DNA corresponding to the variant is then used as the DNA substrate for initiating another round of DNA shuffling. This cycle of shuffling, selecting the optimized variant of interest, and then re-shuffling, is repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages, and can be used to 1) use recombination simultaneously with point mutagenesis; 2) identify beneficial mutations; 3) remove deleterious mutations; and 4) enable parallel processing. DNA shuffling can also be applied to the nucleic acid sequences of the invention (e.g., SEQ ID NO:47-SEQ ID NO:63, SEQ ID NO:67-SEQ ID NO:71, and SEQ ID NO:100-SEQ ID NO:104, or fragments or variants thereof) to decrease the immunogenicity of one or more GPCR-binding peptides in a specified host. For example, a GPCR-binding peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof) or polypeptide may have all of the desired characteristics, but may be highly immunogenic in a host due to its intrinsic structure. Such a limitation can be overcome, for example, by including a copy of the coding sequence of the GPCR-binding peptide or polypeptide with a copy of a xenobiotic coding sequence in one or more cycles of DNA shuffling. Ideally, the resulting hybrid would contain at least some of the coding sequence that enabled the xenobiotic sequence to evade the host immune system, and additionally, the coding sequence of the original binder that provided the desired characteristics.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Vectors and Host Cells

The present invention also encompasses vectors encoding the GPCR-binding peptides (e.g., SEQ ID NO:17-SEQ ID NO:26, SEQ ID NO:35-SEQ ID NO:46, and SEQ ID NO:73-SEQ ID NO:77 or fragments, variants, or derivatives thereof) and host cells comprising these vectors. To express a biologically active GPCR-binding peptide, or its fragments, variants, or derivatives, the nucleotide sequences encoding the amino acid sequence (e.g., SEQ ID NO:47-SEQ ID NO:63, SEQ ID NO:67-SEQ ID NO:71, and SEQ ID NO:100-SEQ ID NO:104), may be inserted into an appropriate expression vector, i.e., a vector, which contains the necessary elements for the transcription and translation of the inserted coding sequence. Methods, which are well known to those skilled in the art, may be used to construct expression vectors containing coding sequences and appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector and host systems may be utilized to contain and express sequences encoding a GPCR-binding peptide (e.g., any one of SEQ ID NO:17-SEQ ID NO:26, SEQ ID NO:35-SEQ ID NO:46, and SEQ ID NO:73-SEQ ID NO:77 or a fragment, variant, or derivative thereof). Such expression systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); and animal cell systems. The present invention is not limited to a specific vector or host system.

“Control elements” or “regulatory sequences” include those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene; La Jolla, Calif.) or PSPORT1 plasmid (Life Technologies), and the like, may be used. The baculovirus polyhedrin promoter may be used in insect cells. Promoters or enhancers derived from the genomes of plant cells (e.g., heat shock, RUBISCO; and storage protein genes), or from plant viruses (e.g., viral promoters or leader sequences), may be cloned into the vector. In mammalian cell systems, promoters from mammalian genes or from mammalian viruses are preferred. If it is necessary to generate a cell line that contains multiple copies of the sequence encoding a GPCR-binding peptide, vectors based on SV40 or EBV may be used with an appropriate selectable marker.

In bacterial systems, a number of expression vectors may be selected, depending upon the use intended for the expressed GPCR binder. For example, when large quantities of expressed protein are needed for the induction of antibodies, vectors, which direct high level expression of fusion proteins that are readily purified, may be used. Such vectors include, but are not limited to, the multifunctional E. coli cloning and expression vectors such as BLUESCRIPT (Stratagene), pIN vectors (see, G. Van Heeke and S. M. Schuster, 1989, J. Biol. Chem., 264:5503-5509); and the like. For a BLUESCRIPT vector, a sequence encoding a GPCR binder (e.g., any one of SEQ ID NO:17-SEQ ID NO:26, SEQ ID NO:35-SEQ ID NO:46, and SEQ ID NO:73-SEQ ID NO:77 or a fragment, variant, or derivative thereof) may be ligated into the vector in-frame with sequences for the amino-terminal methionine and the subsequent 7 residues of β-galactosidase, so that a fusion protein is produced. In addition, pGEX vectors (Promega; Madison, Wis.) may be used to express polypeptides or peptides as glutathione S-transferase-(GST-) fusion proteins. In general, such fusion proteins are soluble and can be easily purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety as desired.

In the yeast, Saccharomyces cerevisiae, a number of vectors containing constitutive or inducible promoters such as alpha factor, alcohol oxidase, and PGH may be used. (For reviews, see F. M. Ausubel et al., supra, and Grant et al., 1987, Methods Enzymol., 153:516-544). Should plant expression vectors be desired and used, the expression of sequences encoding a GPCR-binding peptide may be driven by any of a number of promoters. For example, viral promoters such as the 35S and 19S promoters of CaMV may be used alone or in combination with the omega leader sequence from TMV (N. Takamatsu, 1987, EMBO J, 6:307-311). Alternatively, plant promoters such as the small subunit of RUBISCO, or heat shock promoters, may be used (G. Coruzzi et al., 1984, EMBO J, 3:1671-1680; R. Broglie et al., 1984, Science, 224:838-843; and J. Winter et al., 1991, Results Probl. Cell Differ. 17:85-105). These constructs can be introduced into plant cells by direct DNA transformation or pathogen-mediated transfection. Such techniques are described in a number of generally available reviews (see, for example, S. Hobbs or L. E. Murry, In: McGraw Hill Yearbook of Science and Technology (1992) McGraw Hill, New York, N.Y.; pp. 191-196).

An insect system may also be used to express the GPCR-binding peptides (e.g., SEQ ID NO:17-SEQ ID NO:26, SEQ ID NO:35-SEQ ID NO:46, and SEQ ID NO:73-SEQ ID NO:77 and fragments, variants, and derivatives thereof). For example, Autographa californica nuclear polyhedrosis virus (AcNPV) may be used as a vector to express foreign genes in Spodoptera frugiperda cells or in Trichoplusia larvae. The sequences encoding a GPCR-binding peptide may be cloned into a non-essential region of the virus, e.g., the polyhedrin gene, and placed under control of the polyhedrin promoter. Successful insertion of the foreign coding sequence will render the polyhedrin gene inactive and produce recombinant virus lacking coat protein. The recombinant viruses may then be used to infect, for example, S. frugiperda cells or Trichoplusia larvae (E. K. Engelhard et al., 1994, Proc. Nat. Acad. Sci., 91:3224-3227), and thereby express the GPCR binder.

In mammalian host cells, a number of viral-based expression systems may be utilized. In cases where an adenovirus is used as an expression vector, sequences encoding GPCR-binding peptide may be ligated into an adenovirus transcription/translation complex containing the late promoter and tripartite leader sequence. The coding sequence of a GPCR binder can be inserted into a non-essential E1 or E3 region of the viral genome to yield a viable virus which is capable of expressing the peptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding the GPCR-binding peptides (e.g., SEQ ID NO:47-SEQ ID NO:63, SEQ ID NO:67-SEQ ID NO:71, and SEQ ID NO:100-SEQ ID NO:104, and fragments, variants, and derivatives thereof). In cases where only coding sequence, or a fragment thereof, is inserted, exogenous translational control signals, including a start codon (ATG), should be provided. Furthermore, the start codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system that is used, such as those described in the literature (D. Scharf et al., 1994, Results Probl. Cell Differ., 20:125-162).

To obtain expression in eukaryotic cells, terminator sequences, polyadenylation sequences, and enhancer sequences that modulate gene expression may be required. Sequences that cause amplification of the gene may also be desirable. These sequences are well known in the art. Furthermore, sequences that facilitate secretion of the recombinant product from cells, including, but not limited to, bacteria, yeast, and animal cells, such as secretory signal sequences and/or preprotein or proprotein sequences, may also be included. Such sequences are well described in the art.

Non-limiting examples of suitable host cells include bacteria, archea, insect, fungi (e.g., yeast), plant, and animal cells (e.g., mammalian, especially human). Of particular interest are Escherichia coli, Bacillus subtilis, Saccharomyces cerevisiae, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although it will be appreciated by the skilled practitioner that other cell lines may be used, e.g., to provide higher expression desirable glycosylation patterns, or other features.

Suitable cell-free expression systems for use with this invention include, without limitation, rabbit reticulocyte lysate, wheat germ extract, canine pancreatic microsomal membranes, E. coli S30 extract, and coupled transcription/translation systems (Promega Corp., Madison, Wis.). These systems allow the expression of recombinant polypeptides or peptides upon the addition of cloning vectors, DNA fragments, or RNA sequences containing protein-coding regions and appropriate promoter elements.

Host cells can be transformed, transfected, or infected as appropriate by any suitable method including electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Alternatively, vectors containing the nucleic acids of interest can be transcribed in vitro, and the resulting RNA introduced into the host cell by well-known methods, e.g., by injection (see, Kubo et al., 1988, FEBS Letts. 241:119). The cells into which have been introduced nucleic acids described above are meant to also include the progeny of such cells.

For use with the invention, a host cell strain may be chosen for its ability to modulate the expression of the inserted sequences or to process a GPCR-binding peptide in the desired fashion. Such modifications of the polypeptide include, but are not limited to, acetylation, carboxylation, glycosylation, phosphorylation, lipidation, and acylation. Post-translational processing which cleaves a prepro form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), American Type Culture Collection (ATCC), 10801 University Boulevard, Manassas, Va. 20110-2209, and may be chosen to ensure the correct modification and processing of the amino acid sequence.

For long-term, high-yield production of recombinant GPCR-binding peptides (e.g., SEQ ID NO:17-SEQ ID NO:26, SEQ ID NO:35-SEQ ID NO:46, and SEQ ID NO:73-SEQ ID NO:77 and fragments, variants, and derivatives thereof), stable expression is preferred. For example, cell lines which stably express a GPCR-binding peptide may be transformed using expression vectors, which may contain viral origins of replication and/or endogenous expression elements, and a selectable marker gene on the same, or on a separate, vector. Following the introduction of the vector, cells may be allowed to grow for 1-2 days in an enriched cell culture medium before they are switched to selective medium. The purpose of the selectable marker is to confer resistance to selection, and its presence allows the growth and recovery of cells, which successfully express the introduced sequences. Resistant clones of stably transformed cells may be proliferated using tissue culture techniques appropriate to the cell type.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if a nucleic acid sequence encoding a GPCR-binding peptide is inserted within a marker gene, recombinant cells containing the sequence can be identified by the absence of marker gene function. Alternatively, a marker gene can be placed in tandem with a sequence encoding a GPCR-binding peptide under the control of a single promoter. Expression of the marker gene in response to induction or selection usually indicates co-expression of the tandem gene.

Alternatively, host cells, which contain a nucleic acid sequence encoding a GPCR-binding peptide may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or amino acid sequences. Hybridization assays utilize probes comprising portions or fragments of the nucleic acid sequences (e.g., SEQ ID NO:47-SEQ ID NO:63, SEQ ID NO:67-SEQ ID NO:71, and SEQ ID NO:100-SEQ ID NO:104, and variant sequences). Nucleic acid amplification based assays involve the use of primers, based on the nucleic acid sequences (e.g., SEQ ID NO:47-SEQ ID NO:63, SEQ ID NO:67-SEQ ID NO:71, and SEQ ID NO:100-SEQ ID NO:104, and fragments, variants, and derivatives thereof).

Host cells transformed with nucleotide sequences encoding a GPCR-binding peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof) may be cultured under conditions suitable for the expression and recovery of the amino acid sequence from cell culture. The amino acid sequence may be secreted or contained intracellularly depending on the sequence and/or the vector used. As will be understood by those having skill in the art, expression vectors containing nucleic acid sequences which encode GPCR-binding peptides may be designed to contain signal sequences which direct secretion of the peptides through a prokaryotic or eukaryotic cell membrane. Other constructions may be used to fuse the coding sequences of GPCR binders to epitope or protein tags, which will facilitate purification. Such tags include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp.; Seattle, Wash.). The inclusion of cleavable linker sequences such as those specific for Factor XA or enterokinase (Invitrogen; San Diego, Calif.) between the purification domain and a GPCR-binding peptide may be used to facilitate purification. One such expression vector provides for expression of a fusion protein containing a GPCR-binding peptide and a nucleic acid encoding 6 histidine residues preceding a thioredoxin or an enterokinase cleavage site. The histidine residues facilitate purification on IMAC (immobilized metal ion affinity chromatography) as described by J. Porath et al. (1992, Prot. Exp. Purif, 3:263-281), while the enterokinase cleavage site provides a means for removing the histidine tag sequence. For a discussion of suitable vectors for fusion protein production, see D. J. Kroll et al., 1993; DNA Cell Biol., 12:441-453.

The embodiments of the present invention can be practiced using methods for DNA sequencing which are well known and generally available in the art. The methods may employ such enzymes as the Klenow fragment of DNA polymerase I, SEQUENASE (US Biochemical Corp.; Cleveland, Ohio), Taq polymerase (PE Biosystems), thermostable T7 polymerase (Amersham Pharmacia Biotech; Piscataway, N.J.), or combinations of recombinant polymerases and proofreading exonucleases such as the ELONGASE Amplification System marketed by Life Technologies (Gaithersburg, Md.). Preferably, sequencing is automated with machines such as the Hamilton Micro Lab 2200 (Hamilton, Reno, Nev.), Peltier Thermal Cycler (PTC200; MJ Research; Watertown, Mass.) and the ABI Catalyst and 373 and 377 DNA sequencers (PE Biosystems).

Commercially available capillary electrophoresis systems may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g., GENOTYPER and SEQUENCE NAVIGATOR, PE Biosystems) and the entire process, from loading of samples to computer analysis and electronic data display, may be computer-controlled. Capillary electrophoresis is especially preferable for the sequencing of small pieces of DNA, which may be present in limited amounts in a particular sample.

### Amino Acid Sequences

The present invention encompasses peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof) that bind to HGPRBMY3 and/or other GPC receptors, as well as oligopeptides, and polypeptides comprising these peptides. In accordance with the invention, amino acid sequence fragments can range in size from 5 amino acid residues to all but one residue of the entire sequence. Accordingly, amino acid sequence fragments include, but are not limited to, fragments comprising at least 5, 6, 7, 8, 9, 10, 11, 12, 13, 14, or 15 contiguous amino acids of any one of SEQ ID NO:17-SEQ ID NO:26, SEQ ID NO:35-SEQ ID NO:46, and SEQ ID NO:73-SEQ ID NO:77. Preferably, fragments comprise at least 7, 8, or 9 contiguous amino acids of any one of SEQ ID NO:17-SEQ ID NO:26, SEQ ID NO:35-SEQ ID NO:46, and SEQ ID NO:73-SEQ ID NO:77. More preferably, the fragments comprise one or more binding motifs, including KIW, KVW, KLW, RVW, and TPHRVWXLP, wherein X is Q or N.

The amino acid sequences of the invention also encompass peptide, oligopeptide, and polypeptide sequences that comprise at least one, at least two, at least three, or at least four P2Y binding motifs selected from the group consisting of: KIW, KVW, KLW, and RVW. One or more of these motifs may be contiguously linked together (e.g., KIWKVMKLW, etc.) in any order to form a single peptide or polypeptide sequence. Alternatively, one or more of these P2Y binding motifs may be linked together by one or more linker sequences of varying length. Such linker sequences may be synthetic linkers, or even amino acid sequences of variable length and sequence (e.g., KIWXₙKVM, wherein “X” equals any naturally occurring amino acid and “n” equals the number of amino acids, etc.). The number of intervening amino acids is variable, but preferably may be at least 1, 2, 3, 4, 5, 6, 7, 8, 9, 10, 15, 20, 25, 30, 35, 40, or at least 50 amino acids between each motif terminus. Additional examples of linkers encompassed by the present invention are described in U.S. Pat. No. 5,073,627, hereby incorporated by reference.

In one aspect, a peptide or polypeptide of the invention comprises the sequence X(n) NNN X(y−n), where n=number of residues preceding the three residues of the tripeptide motif, y=number of residues in peptide minus 3, X=any codon, and the tripeptide NNN is encoded by any of the codons shown in the table, below. In this table, B=A, C, or T; X=G, A, C, or T; and O=G or A.

| Triplet NNN | Codon 1 | Codon 2 | Codon 3 |
|---|---|---|---|
| RVW | CGX/AGO | GTX | UGG |
| KVW | AAO | GTX | UGG |
| KLW | AAO | CTX/TTO | UGG |
| KIW | AAO | ATB | UGG |

Peptides, oligopeptides, and polypeptides comprising more than one P2Y binding motif may be generated using chemical techniques known in the art. For example, amino acid sequences comprising more than one P2Y binding motif (referred to herein as a multimer) may be created by chemically cross-linking two or more peptides (referred to herein as subunits) comprising one or more P2Y binding motifs, using linker molecules and linker molecule length optimization techniques known in the art. See, e.g., U.S. Pat. No. 5,478,925, which is herein incorporated by reference in its entirety. Additionally, multimers of the invention may be generated using techniques known in the art to form one or more inter-molecule cross-links between the cysteine residues of the subunits to be included in the multimer (see, e.g., U.S. Pat. No. 5,478,925, which is herein incorporated by reference in its entirety). Further, a peptide and polypeptide of the invention may be routinely modified by the addition of cysteine or biotin to the C-terminus or N-terminus of the sequences. Following this, techniques known in the art may be used to generate multimers containing one or more of these modified sequences. See, e.g., U.S. Pat. No. 5,478,925, which is herein incorporated by reference in its entirety. Additionally, techniques known in the art may be used to generate liposomes containing the subunits to be included in the multimers (see, e.g., U.S. Pat. No. 5,478,925, which is herein incorporated by reference in its entirety).

Another method for preparing multimers of the invention involves use of the peptides or polypeptides of the invention fused to a leucine zipper or isoleucine zipper domain. Leucine zipper and isoleucine zipper domains are sequences that promote multimerization of the polypeptides in which they are found. Leucine zippers were originally identified in several DNA-binding proteins (Landschulz et al., Science 240:1759, (1988)), but have since been found in a variety of proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Peptides, oligopeptides, and polypeptides comprising one or more P2Y-binding motifs may also be linked to other proteins, or even short marker peptides (e.g., epitope tags, HA, GST, thioredoxin, maltose binding protein, etc.). Components that may be linked to the subunits or multimers of the invention include immunoglobulins, Fc fragments, therapeutic proteins, cytotoxic proteins, small molecules, polysaccarides, lipids, proteins containing a binding motif, etc. For example, a P2Y-binding motif containing amino acid sequence may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Non-limiting examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response. The therapeutic agent or drug moiety is not to be construed as limited to classical chemical therapeutic agents. For example, the drug moiety may be a peptide or polypeptide possessing a desired biological activity. Such components may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (see, International Publication No. WO 97/34911), Fas ligand (Takahashi et al., Int. Immunol., 6:1567-1574 (1994)), VEGI (see, International Publication No. WO 99/23105), a thrombotic agent and an anti-angiogenic agent, e.g., angiostatin or endostatin; and, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (“G-CSF”), and other growth factors.

Also provided by the invention are chemically modified derivatives of the peptides and polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The invention further encompasses chemical derivitization of the polypeptides of the present invention, preferably where the chemical is a hydrophilic polymer residue. Exemplary hydrophilic polymers, including derivatives, may be those that include polymers in which the repeating units contain one or more hydroxy groups (polyhydroxy polymers), including, for example, poly(vinyl alcohol); polymers in which the repeating units contain one or more amino groups (polyamine polymers), including, for example, peptides, polypeptides, proteins and lipoproteins, such as albumin and natural lipoproteins; polymers in which the repeating units contain one or more carboxy groups (polycarboxy polymers), including, for example, carboxymethylcellulose, alginic acid and salts thereof, such as sodium and calcium alginate, glycosaminoglycans and salts thereof, also including salts of hyaluronic acid, phosphorylated and sulfonated derivatives of carbohydrates, genetic material, such as interleukin-2 and interferon, and phosphorothioate oligomers; and polymers in which the repeating units contain one or more saccharide moieties (polysaccharide polymers), including, for example, carbohydrates.

The molecular weight of the hydrophilic polymers may vary, and is generally about 50 to about 5,000,000, with polymers having a molecular weight of about 100 to about 50,000 being preferred. The polymers may be branched or unbranched. More preferred polymers have a molecular weight of about 150 to about 10,000, with molecular weights of 200 to about 8,000 being even more preferred. For polyethylene glycol, the preferred molecular weight is between about 1 kDa and about 100 kDa for ease in handling and manufacturing. The term “about” indicates that in preparations of polyethylene glycol, some molecules will weigh more, some less, than the stated molecular weight. Other sizes may be used, depending on the desired therapeutic profile, e.g., the duration of sustained release desired, the effects, if any on biological activity, the ease in handling, the degree or lack of antigenicity and other known effects of the polyethylene glycol to a therapeutic protein or analog.

Additional preferred polymers that may be used to derivatize polypeptides of the invention, include, for example, poly(ethylene glycol) (PEG), poly(vinylpyrrolidine), polyoxomers, polysorbate and poly(vinyl alcohol), with PEG polymers being particularly preferred. Preferred among the PEG polymers are PEG polymers having a molecular weight of from about 100 to about 10,000. More preferably, the PEG polymers have a molecular weight of from about 200 to about 8,000, with PEG 2,000, PEG 5,000 and PEG 8,000, which have molecular weights of 2,000, 5,000 and 8,000, respectively, being even more preferred. Other suitable hydrophilic polymers, in addition to those exemplified above, will be readily apparent to one skilled in the art based on the present disclosure. Generally, the polymers used may include polymers that can be attached to the polypeptides of the invention via alkylation or acylation reactions.

The polyethylene glycol molecules (or other chemical moieties) should be attached to the protein with consideration of effects on functional or antigenic domains of the protein. There are a number of attachment methods available to those skilled in the art, for example, PEG coupling (see, e.g., EP 0 401 384, herein incorporated by reference), and pegylation of using tresyl chloride (see, also Malik et al., Exp. Hematol. 20:1028-1035 (1992)). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and N-terminal residues. Those having a free carboxyl group may include aspartic acid residues, glutamic acid residues, and the C-terminal residues. Sulfhydryl groups may also be used as a reactive group for attaching the polyethylene glycol molecules. Preferred for therapeutic purposes is attachment at an amino group, such as attachment at the N-terminus or a lysine group.

For some purposes, peptides and polypeptides can be chemically modified at the N-terminus. Using polyethylene glycol as an illustration of the present composition, one may select from a variety of polyethylene glycol molecules (by molecular weight, branching, etc.), the proportion of polyethylene glycol molecules to peptide or polypeptide molecules in the reaction mix, the type of pegylation reaction to be performed, and the method of obtaining the selected N-terminally pegylated product. The method of obtaining the N-terminally pegylated preparation (i.e., separating this moiety from other monopegylated moieties if necessary) may be by purification of the N-terminally pegylated material from a population of pegylated products. Selective modification at the N-terminus may be accomplished by reductive alkylation which exploits differential reactivity of different types of primary amino groups (lysine versus the N-terminus) available for derivatization in a particular peptide or polypeptide. Under the appropriate reaction conditions, substantially selective derivatization of the protein at the N-terminus with a carbonyl group containing polymer is achieved.

As with the various polymers exemplified above, it is contemplated that the polymeric residues may contain functional groups in addition, for example, to those typically involved in linking the polymeric residues to the polypeptides of the present invention. Such functionalities include, for example, carboxyl, amine, hydroxy, and thiol groups. Functional groups on the polymeric residues can be further reacted, if desired, with materials that are generally reactive with such functional groups and which can assist in targeting specific tissues in the body including, for example, diseased tissue. Exemplary materials that can be reacted with the additional functional groups include, for example, proteins, and antibodies, carbohydrates, peptides, glycopeptides, glycolipids, lectins, and nucleosides.

In addition to residues of hydrophilic polymers, the chemical used to derivatize the polypeptides of the present invention can be a saccharide residue. Exemplary saccharides which can be derived include, for example, monosaccharides or sugar alcohols, such as erythrose, threose, ribose, arabinose, xylose, lyxose, fructose, sorbitol, mannitol and sedoheptulose, with preferred monosaccharides being fructose, mannose, xylose, arabinose, mannitol and sorbitol; and disaccharides, such as lactose, sucrose, maltose and cellobiose. Other saccharides include, for example, inositol and ganglioside head groups. Other suitable saccharides, in addition to those exemplified above, will be readily apparent to one skilled in the art based on the present disclosure. Generally, saccharides that may be used for derivitization include saccharides that can be attached to the polypeptides of the invention via alkylation or acylation reactions.

Moreover, the invention also encompasses derivitization of the peptides and polypeptides of the present invention, for example, with lipids (including cationic, anionic, polymerized, charged, synthetic, saturated, unsaturated, and any combination of the above, etc.). The invention further encompasses the derivitization of the peptides and polypeptides of the present invention, for example, with compounds that may serve a stabilizing function, e.g., to increase the polypeptides half-life in solution, to make the polypeptides more water soluble, to increase the polypeptides hydrophilic or hydrophobic character, etc. Polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof.

Suitable polymers include, but are not limited to, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, and polyethylenes (such as, for example, polyethylene glycol (including for example, the class of compounds referred to as Pluronics.RTM., commercially available from BASF, Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof.

Methods for the preparation of derivatized peptides and polypeptides of the invention which employ polymers as stabilizing compounds will be readily apparent to one skilled in the art, in view of the present disclosure and information known in the art, such as that described and referred to in Unger, U.S. Pat. No. 5,205,290, which is hereby incorporated by reference herein in its entirety. The invention encompasses additional modifications of the polypeptides of the present invention. Such additional modifications are known in the art, and are specifically provided, in addition to methods of derivitization, etc., in U.S. Pat. No. 6,028,066, which is hereby incorporated in its entirety herein.

In addition, amino acid sequence variants of the present invention include, but are not limited to, variants that share at least 40%, 50%, 60%, 61%, 67%, 70%, 74%, 76%, 80%, 81%, 84%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% nucleotide sequence identity with any one of SEQ ID NO:17-SEQ ID NO:26, SEQ ID NO:35-SEQ ID NO:46, and SEQ ID NO:73-SEQ ID NO:77. For variants that are functional equivalents, the percent amino acid sequence identity is at least 61% or 67%. More preferably, the percent amino acid sequence identity is at least 74% or 76%, still more preferably, at least 81% or 84%, and even more preferably, at least 90% to any one of SEQ ID NO:17-SEQ ID NO:26, SEQ ID NO:35-SEQ ID NO:46, and SEQ ID NO:73-SEQ ID NO:77. However, as will be appreciated by the skilled practitioner, biological function per se need not be maintained where an amino acid fragment comprises an antigenic epitope.

Polypeptide and peptide variants include variants differing by the addition, deletion, or substitution of one or more amino acid residues. For example, for screening for GPCR modulating agents, it may be useful to encode a tagged GPCR-binding peptide or polypeptide that can be recognized by a commercially available antibody. For example, a peptide or polypeptide can be fused or linked to epitope tags (e.g., FLAG, HA, GST, thioredoxin, maltose binding protein, etc.), or affinity tags such as biotin and/or streptavidin. The peptides or polypeptides of the invention can be covalently attached to chemical moieties via the amino acid backbone. For these purposes, the peptides or polypeptides may be modified by N- or C-terminal processing of the sequences (e.g., proteolytic processing), deletion of the N-terminal methionine residue, etc. A peptide or polypeptide tagged with an epitope or protein may also be engineered to contain a cleavage site located between the binder coding sequence and the tag coding sequence. This can be used to remove the tag, and isolate the GPCR-binding peptide or polypeptide. The polypeptides may also be modified with a detectable label, such as an enzymatic, fluorescent, isotopic or affinity label to allow for detection and isolation of the protein, as described in detail herein.

Also included are modified polypeptides and peptides in which one or more residues are modified, and mutants comprising one or more modified residues. The peptides and polypeptides may be differentially modified during or after translation, e.g., by derivatization with known protecting/blocking groups, proteolytic cleavage, linkage to an antibody molecule or other cellular ligand, etc. Useful modifications may include glycosylation, amidation, phosphorylation, sulfation, reduction/alkylation (Tarr, 1986, Methods of Protein Microcharacterization, J. E. Silver, Ed., Humana Press, Clifton, N. J., pp. 155-194); acylation (Tarr, supra); chemical coupling (Mishell and Shiigi (Eds), 1980, Selected Methods in Cellular Immunology, W H Freeman, San Francisco, Calif.; U.S. Pat. No. 4,939,239); and mild formalin treatment (Marsh, 1971, Int. Arch. of Allergy and Appl. Immunol. 41:199-215). Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc. Additional post-translational modifications encompassed by the invention include, for example, e.g., attachment of N-linked or O-linked carbohydrate chains, processing of N-terminal or C-terminal ends, chemical modifications of N-linked or O-linked carbohydrate chains, and addition or deletion of an N-terminal methionine residue as a result of prokaryotic host cell expression.

Additionally, D-amino acids, non-natural amino acids, or non-amino acid analogs can be substituted or added to produce a modified polypeptide. Furthermore, the polypeptides disclosed herein can be modified using polyethylene glycol (PEG) according to known methods (S. I. Wie et al., 1981, Int. Arch. Allergy Appl. Immunol. 64(1):84-99) to produce a protein conjugated with PEG. In addition, PEG can be added during chemical synthesis of the protein. Modifications or sequence variations may occur at the amino- or carboxy-terminal positions of the reference polypeptide sequence or anywhere between those terminal positions, interspersed either individually among the amino acids in the reference sequence or in one or more contiguous groups within the reference sequence. The polypeptides and peptides of this invention can be isolated, synthetic, or recombinant. The amino acid sequences may be obtained as individual polypeptides or peptides, or part of a complex. In various aspects, a complex may comprise one or more GPCR-binding peptides in association with one or more molecules of HGPRBMY3 or a related GPC receptor (e.g., multiple copies of the same peptide or single copies of different peptides).

Polypeptides or peptides may also be modified with a label capable of providing a detectable signal, either directly or indirectly, including, but not limited to, radioisotope, fluorescent, and enzyme labels. Fluorescent labels include, for example, Cy™3, Cy™5, Alexa, BODIPY, fluorescein (e.g., Fluor X, DTAF, and FITC), rhodamine (e.g., TRITC), auramine, Texas Red, AMCA blue, and Lucifer Yellow. Preferred isotope labels include ³H, ¹⁴ C, ³² P, ³⁵ S. ³⁶ Cl, ⁵¹ Cr, ⁵⁷ Co, ⁵⁸ Co, ⁵⁹ Fe, ⁹⁰ Y, ¹²⁵ I, ¹³¹ I, and ¹⁸⁶ Re. Preferred enzyme labels include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, Tyramide Signal Amplification (TSA™), are known in the art, and are commercially available (see, e.g., ABC kit, Vector Laboratories, Inc., Burlingame, Calif.; NEN® Life Science Products, Inc., Boston, Mass.).

GPCR-binding peptides (e.g., SEQ ID NO:17-SEQ ID NO:26, SEQ ID NO:35-SEQ ID NO:46, and SEQ ID NO:73-SEQ ID NO:77 and fragments, variants, and derivatives thereof) may be produced by direct peptide synthesis using solid-phase techniques (J. Merrifield, 1963, J. Am. Chem. Soc., 85:2149-2154; J. Y. Roberge et al., 1995, Science, 269:202-204). Protein or peptide synthesis may be performed using manual techniques or by automation. Automated synthesis may be achieved, for example, using ABI 431 A Peptide Synthesizer (PE Biosystems). Various fragments of a GPCR-binding polypeptide or peptide can be chemically synthesized separately and then combined using chemical methods to produce the full-length molecule. The newly synthesized peptide can be substantially purified by preparative high performance liquid chromatography (e.g., T. Creighton, 1983, Proteins, Structures and Molecular Principles, W. H. Freeman and Co., New York, N. Y.), by reversed-phase high performance liquid chromatography, or other purification methods as are known in the art. The composition of the synthetic peptides may be confirmed by amino acid analysis or sequencing (e.g., the Edman degradation procedure; Creighton, supra). In addition, the amino acid sequence of GPCR-binding peptide or polypeptide or any portion thereof, may be altered during direct synthesis and/or combined using chemical methods with sequences from other proteins, or any part thereof, to produce a variant peptide or polypeptide.

Peptides and polypeptides may possess characteristics that make them undesirable for transgenic, therapeutic, pharmaceutical, and/or industrial applications. For example, some peptides and polypeptides possess a short physiological half-life. This problem may present either at the level of the protein, or the level of the mRNA. The ability to extend the half-life, for example, is particularly important for using proteins in gene therapy, transgenic animal production, the bioprocess production and purification of the peptide or polypeptide, and as a chemical modulator among others. Therefore, there is a need to identify novel variants of the peptides (e.g., SEQ ID NO:17-SEQ ID NO:26, SEQ ID NO:35-SEQ ID NO:46, and SEQ ID NO:73-SEQ ID NO:77 or derivatives or fragments thereof), or polypeptides comprising these peptides, which possess characteristics that enhance their use in diagnostic, therapeutic, and industrial applications.

In one aspect, peptides and polypeptides may be enhanced by directed molecular evolution (also called DNA shuffling), as described in detail herein. As examples, alterations may be made to enhance or optimize solubility, structure, or codon usage, specific biological activity, including any associated enzymatic activity, enzyme kinetics, Ki, Kcat, Km, Vmax, Kd, protein-protein or protein-peptide activity, binding activity, antagonist/inhibitory activity (including direct or indirect interaction), agonist activity (including direct or indirect interaction), antigenicity (e.g., where it would be desirable to either increase or decrease the antigenic potential), immunogenicity, formation of dimers, trimers, or multimers, and efficacy, including its subsequent use as a preventative or treatment for diseases or disease states, or as an effector for targeting disease agents. Moreover, the specific characteristics of a peptide or polypeptide may also be changed to produce an activity completely unrelated to its initially characterized activity. Other desirable alterations of a peptide or polypeptide would be specific to each individual amino acid sequence, and could be achieved using well-known methods in the art

In one aspect, a GPCR-binding peptide or polypeptide could be altered to cause constitutive activation of HGPRBMY3 or another GPC receptor. Upon constitutive activation, the receptor may be capable of being activated in the absence of its cognate ligand, or with less than all of the regulatory factors and/or conditions typically required for GPC receptor activation (e.g., ligand binding, phosphorylation, conformational changes, etc.). Activated HGPRBMY3 or another GPC receptor could be used in screens to identify modulators, among other uses described herein. Alternatively, a GPCR-binding peptide or polypeptide could be altered to inactivate HGPRBMY3 or another GPC receptor.

In one embodiment of this invention, one or more GPCR-binding peptides (e.g., SEQ ID NO:17-SEQ ID NO:26, SEQ ID NO:35-SEQ ID NO:46, and SEQ ID NO:73-SEQ ID NO:77 or variants or fragments thereof), polypeptides, and anti-idiotype antibodies may be used as reagents to isolate, identify, quantify, or localize human HGPRBMY3, or other GPC receptors. For example, GPCR-binding peptides, polypeptides, or antibodies can be used to form GPCR/binder complexes, and thereby isolate or purify the cognate GPC receptors. In particular, GPC receptors such as HGPRBMY3 can be precipitated by labeled binder peptides, polypeptides, or antibodies. Alternatively, GPC receptors (e.g., HGPRBMY3) can be isolated by binder peptides, polypeptides, or antibodies affixed to a solid surface, for example, resin, beads, microtiter plates, etc. As another example, labeled binder peptides, polypeptides, or antibodies can be used in protein blotting experiments to determine the presence, absence, or expression levels of HGPRBMY3 or another GPC receptor. In addition, labeled binders can be used in histological experiments, to localize HGPRBMY3 or other GPC receptors in cells or tissues.

In other embodiments, one or more GPCR-binding peptides (e.g., SEQ ID NO:17-SEQ ID NO:26, SEQ ID NO:35-SEQ ID NO:46, and SEQ ID NO:73-SEQ ID NO:77 or fragments, variants, or derivatives thereof), polypeptides, and anti-idiotype antibodies may be useful as diagnostic, prophylactic, and therapeutic agents for immunological disorders, immunodeficiency diseases, immune reactions to transplanted organs and tissues, autoimmunity disorders, hypersensitivities, proliferative disorders, neurological disorders, dyskinesias, and infection susceptibility. Proliferative diseases and/or disorders include proliferative conditions within immune cells and tissues, which include but are not limited to proliferative conditions of the lymph node, spleen, hematopoietic system; proliferative conditions of the renal system, which include but are not limited to proliferative conditions of the kidney; proliferative conditions of reproductive cells and tissues, which include but are not limited to proliferative conditions of the breast, ovary, cervix, uterus, and prostate. Also included are proliferative disorders of the respiratory system, including proliferative conditions of the lungs; proliferative disorders of the glandular system, including proliferative conditions of the adrenal, thyroid, and salivary glands; proliferative conditions of the gastrointestinal system, including proliferative conditions of the stomach and colon; and proliferative disorders of the skin or connective tissues. Specifically included are leukemia and cancers of the breast, ovary, uterus, cervix, prostate, lung, skin, and colon, as well as particular disorders such as melanoma, squamous cell carcinoma, and fibrosarcoma.

Other diseases and disorders include, but are not limited to, arthritis, rheumatoid arthritis, asthma, leukemia, granulomatous disease, inflammatory bowel disease, sepsis, allergies, acne, neutropenia, neutrophilia, psoriasis, AIDS, T-cell mediated cytotoxicity; host-versus-graft and graft-versus-host diseases, autoimmune infertility, lense tissue injury, demyelination, systemic lupus erythematosis, drug-induced hemolytic anemia, Sjogren's disease, scleroderma, multiple myeloma, B-cell neoplasms, T-cell neoplasms, cerebral neoplasms, Hodgkin's disease, lymphoma, follicular lymphoma, splenic marginal zone lymphoma, nodal marginal zone lymphoma, mantle cell lymphoma, hairy cell leukemia, prolymphocytic leukemia (B cell or T cell), lymphoplasmacytic lymphoma, Sézary syndrome, smoldering adult T cell leukemia/lymphoma, Burkitt's lymphoma, post-organ transplant lymphoma, Castleman's disease, Rosai-Dorfinan's disease, lymphomatoid papulosis, non-Hodgkin's lymphoma, and benign prostatic hypertrophy.

Additional non-limiting examples of diseases and disorders include anorexia, bulimia, acute heart failure, hypotension, hypertension, urinary retention, osteoporosis, angina pectoris, myocardial infarction, ulcers, tardive dyskinesia, dystonias, epilepsy, akathesia, multiple sclerosis, amyotrophic lateral sclerosis,. Parkinson's disease, anxiety, bipolar disorder, catatonia, schizophrenia, manic depression, delirium, dementia, paranoid psychoses, severe mental retardation, Down's syndrome, Alzheimer's disease, amnesia, Huntington's disease, Gilles dela Tourett's syndrome, increased susceptibility to EPV infection, increased susceptibility to HIV (e.g., HIV-1 or HIV-2) infection, increased susceptibility to herpes viral infections, and increased susceptibility to H. pylori infections. The GPCR-binding peptides or polypeptides may also be useful for modulating cytokine production, antigen presentation, or other processes, such as for boosting immune responses, etc.

In various aspects, the peptides (e.g., SEQ ID NO:17-SEQ ID NO:26, SEQ ID NO:35-SEQ ID NO:46, and SEQ ID NO:73-SEQ ID NO:77 and fragments, variants, and derivatives thereof), polypeptides, and anti-idiotype antibodies of the invention may specifically modulate and/or bind to HGPRBMY3. Alternatively, the peptides, polypeptides, and antibodies may also modulate and/or bind to other GPC receptors, for example, purinergic, P2Y receptors, P2Y-like receptors, and somatostatin receptors, including, but not limited to, human GPR68 (SEQ ID NO:8); human BRGR1B receptor (SEQ ID NO:9); human P2Y5-like receptor (SEQ ID NO:10); human P2Y9 receptor (SEQ ID NO:11); chicken P2Y5 receptor (SEQ ID NO:12); human P2Y5 receptor (SEQ ID NO:13); human GPR17 receptor (SEQ ID NO:14); a rat orphan GPC receptor (SEQ ID NO:15); and human SSR4 receptor (SEQ ID NO:16). The skilled artisan would recognize the potential for the GPCR-binding peptides of the present invention to modulate the activity of other P2Y receptors known in the art.

### Sequence Analysis

Those having skill in the art will know how to determine percent identity between or among sequences using, for example, algorithms such as those based on the CLUSTALW computer program (J. D. Thompson et al., 1994, Nucleic Acids Res., 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the percent identity. CLUSTALW, however, does take sequence gaps into account in its identity calculations.

As a practical matter, whether any particular nucleic acid molecule or polypeptide is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleic acid sequence of the present invention (e.g., SEQ ID NO:47-SEQ ID NO:63, SEQ ID NO:67-SEQ ID NO:71, and SEQ ID NO:100-SEQ ID NO:104) can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992).

In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k−tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multiple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched, the final percent identity would be 90%. In another example, a 90 base subject sequence is compared with a 100 base query sequence. This time, the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

As a practical matter, whether any particular polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to an amino acid sequence of the invention (e.g., SEQ ID NO:17-SEQ ID NO:26, SEQ ID NO:35-SEQ ID NO:46, and SEQ ID NO:73-SEQ ID NO:77) can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992).

In a sequence alignment the query and subject sequences are both amino acid sequences. The result of said global sequence alignment is in percent identity. Preferred parameters used in a CLUSTALW alignment of polypeptide sequences to calculate percent identity via pairwise alignments are: Matrix=BLOSUM, k−tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty-10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multiple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global amino acid sequence alignments are often preferred since they reflect the percent identity between the peptide or polypeptide molecules as a whole (i.e., including any sequence overhangs, not just overlapping regions), as opposed to, only local matching polypeptides. Manual corrections for global percent identity determinations are required since the CLUSTALW program does not account for N- and C-terminal truncations of the subject sequence when calculating percent identity.

For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more nucleic acid or amino acid sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modified CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

Where a peptide or polypeptide has an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a subject amino acid sequence having at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

### Peptide Libraries

Peptide libraries (e.g., secondary libraries) produced and screened according to this invention are useful for identifying new ligands for HGPRBMY3 or related GPC receptors. In particular, GPCR-binding peptides (e.g., SEQ ID NO:17-SEQ ID NO:26, SEQ ID NO:35-SEQ ID NO:46, and SEQ ID NO:73-SEQ ID NO:77), nucleic acids encoding these peptides, or variants or fragments thereof, can be used to generate peptide libraries in accordance with known techniques. Peptide libraries can be designed and panned according to methods described in detail herein, and methods generally available to those in the art (see, e.g., U.S. Pat. No. 5,723,286 issued Mar. 3, 1998 to Dower et al.). In one aspect, commercially available phage display libraries can be used (e.g., Ph.D. C7C Disulfide Constrained Peptide Library, NEB). In another aspect, an oligonucleotide library can be prepared according to methods known in the art, and inserted into an appropriate vector for peptide expression. For example, vectors encoding a bacteriophage structural protein, preferably an accessible phage protein, such as a bacteriophage coat protein, can be used. Although one skilled in the art will appreciate that a variety of bacteriophage may be employed in this invention, in preferred embodiments the vector is, or is derived from, a filamentous bacteriophage, such as, for example, fl, fd, Pfl, M13, etc. In particular, the fd-tet vector has been extensively described in the literature (see, e.g., Zacher et al., 1980, Gene 9:127-140; Smith et al., 1985, Science 228:1315-1317; Parmley and Smith, 1988, Gene 73:305-318).

The phage vector is chosen to contain or is constructed to contain a cloning site located in the 5′ region of the gene encoding the bacteriophage structural protein, so that the peptide is accessible to receptors in an affinity enrichment procedure as described herein below. The structural phage protein is preferably a coat protein. An example of an appropriate coat protein is pIII. A suitable vector may allow oriented cloning of the oligonucleotide sequences that encode the peptide so that the peptide is expressed at or within a distance of about 100 amino acid residues of the N-terminus of the mature coat protein. The coat protein is typically expressed as a preprotein, having a leader sequence.

Thus, desirably the oligonucleotide library is inserted so that the N-terminus of the processed bacteriophage outer protein is the first residue of the peptide, i.e., between the 3′-terminus of the sequence encoding the leader protein and the 5′-terminus of the sequence encoding the mature protein or a portion of the 5′ terminus. The library is constructed by cloning an oligonucleotide which contains the variable region of library members (and any spacers, as discussed below) into the selected cloning site. Using known recombinant DNA techniques (see generally, Sambrook et al., 1989, Molecular Cloning, A Laboratory Manual, 2d ed., Cold Spring Harbor Laboratory Press, Cold Spring Harbor, N.Y., 1989), an oligonucleotide may be constructed which, inter alia; 1) removes unwanted restriction sites and adds desired ones; 2) reconstructs the correct portions of any sequences which have been removed (such as a correct signal peptidase site, for example); 3) inserts the spacer residues, if any; and/or 4) corrects the translation frame (if necessary) to produce active, infective phage.

The central portion of the oligonucleotide will generally contain one or more GPCR-binding sequences and, optionally, spacer sequences. The sequences are ultimately expressed as peptides (with or without spacers) fused to or in the N-terminus of the mature coat protein on the outer, accessible surface of the assembled bacteriophage particles. The size of the library will vary according to the number of variable codons, and hence the size of the peptides, which are desired. Generally the library will be at least about 10⁶ members, usually at least 10⁷, and typically 10⁸ or more members. To generate the collection of oligonucleotides which forms a series of codons encoding a random collection of amino acids and which is ultimately cloned into the vector, a codon motif is used, such as (NNK)ₓ, where N may be A, C, G, or T (nominally equimolar), K is G or T (nominally equimolar), and x is typically up to about 5, 6, 7, 8, or more, thereby producing libraries of penta-, hexa-, hepta-, and octa-peptides or larger. The third position may also be G or C, designated “S”. Thus, NNK or NNS 1) code for all the amino acids; 2) code for only one stop codon; and 3) reduce the range of codon bias from 6:1 to 3:1.

It should be understood that, with longer peptides, the size of the library that is generated may become a constraint in the cloning process. The expression of peptides from randomly generated mixtures of oligonucleotides in appropriate recombinant vectors is known in the art (see, e.g., Oliphant et al., Gene 44:177-183). For example, the codon motif (NNK)₆ produces 32 codons, one for each of 12 amino acids, two for each of five amino acids, three for each of three amino acids and one (amber) stop codon. Although this motif produces a codon distribution as equitable as available with standard methods of oligonucleotide synthesis, it results in a bias against peptides containing one-codon residues. In particular, a complete collection of hexacodons contains one sequence encoding each peptide made up of only one-codon amino acids, but contains 729 (3⁶) sequences encoding each peptide with only three-codon amino acids.

An alternative approach to minimize the bias against one-codon residues involves the synthesis of 20 activated trinucleotides, each representing the codon for one of the 20 genetically encoded amino acids. These are synthesized by conventional means, removed from the support while maintaining the base and 5-OH-protecting groups, and activated by the addition of 3′O-phosphoramidite (and phosphate protection with b-cyanoethyl groups) by the method used for the activation of mononucleosides (see, generally, McBride and Caruthers, 1983, Tetrahedron Letters 22:245). Degenerate oligocodons are prepared using these trimers as building blocks. The trimers are mixed at the desired molar ratios and installed in the synthesizer. The ratios will usually be approximately equimolar, but may be a controlled unequal ratio to obtain the over- to under-representation of certain amino acids coded for by the degenerate oligonucleotide collection. The condensation of the trimers to form the oligocodons is done essentially as described for conventional synthesis employing activated mononucleosides as building blocks (see, e.g., Atkinson and Smith, 1984, Oligonucleotide Synthesis, M. J. Gait, Ed., p. 35-82). This procedure generates a population of oligonucleotides for cloning that is capable of encoding an equal distribution (or a controlled unequal distribution) of the possible peptide sequences. Advantageously, this approach may be employed in generating longer peptide sequences, since the range of bias produced by the (NNK)₆ motif increases by three-fold with each additional amino acid residue.

When the codon motif is (NNK)ₓ, as defined above, and when x equals 8, there are 2.6.×10¹⁰ possible octa-peptides. A library containing most of the octa-peptides may be difficult to produce. Thus, a sampling of the octa-peptides may be accomplished by constructing a subset library using up to about 10% of the possible sequences, which subset of recombinant bacteriophage particles is then screened. If desired, to extend the diversity of a subset library, the recovered phage subset may be subjected to mutagenesis and then subjected to subsequent rounds of screening. This mutagenesis step may be accomplished in two general ways: the variable region of the recovered phage may be mutagenized, or additional variable amino acids may be added to the regions adjoining the initial variable sequences.

To diversify around active peptides (i.e., GPCR binders) found in early rounds of panning, the positive phage can be sequenced to determine the identity of the active peptides. Oligonucleotides can then be synthesized based on these peptide sequences. The syntheses are done with a low level of all bases incorporated at each step to produce slight variations of the primary oligonucleotide sequences. This mixture of (slightly) degenerate oligonucleotides can then be cloned into the affinity phage by methods known to those in the art. This method produces systematic, controlled variations of the starting peptide sequences as part of a secondary library. It requires, however, that individual positive phage be sequenced before mutagenesis, and thus is useful for expanding the diversity of small numbers of recovered phage.

An alternate approach to diversify the selected phage allows the mutagenesis of a pool, or subset, of recovered phage. In accordance with this approach, phage recovered from panning are pooled and single stranded DNA is isolated. The DNA is mutagenized by treatment with, e.g., nitrous acid, formic acid, or hydrazine. These treatments have different damaging effects on the DNA. The damaged DNA is then copied with reverse transcriptase, which misincorporates bases when it encounters a site of damage. The segment containing the sequence encoding the receptor-binding peptide is then isolated by cutting with restriction nuclease(s) specific for sites flanking the peptide coding sequence. This mutagenized segment is then recloned into undamaged vector DNA, the DNA is transformed into cells, and a secondary library according to known methods. General mutagenesis methods are known in the art (see Myers et al., 1985, Nucl. Acids Res. 13:3131-3145; Myers et al., 1985, Science 229:242-246; Myers, 1989, Current Protocols in Molecular Biology Vol I, 8.3.1-8.3.6, F. Ausubel et al., eds, J. Wiley and Sons, New York).

In another general approach, the addition of amino acids to a peptide or peptides found to be active, can be carried out using various methods. In one, the sequences of peptides selected in early panning are determined individually and new oligonucleotides, incorporating the determined sequence and an adjoining degenerate sequence, are synthesized. These are then cloned to produce a secondary library. Alternatively, methods can be used to add a second GPCR-binding sequence to a pool of peptide-bearing phage. In accordance with one method, a restriction site is installed next to the first GPCR-binding sequence. Preferably, the enzyme should cut outside of its recognition sequence. The recognition site may be placed several bases from the first binding sequence. To insert a second GPCR-binding sequence, the pool of phage DNA is digested and blunt-ended by filling in the overhang with Klenow fragment. Double-stranded, blunt-ended, degenerately synthesized oligonucleotides are then ligated into this site to produce a second binding sequence juxtaposed to the first binding sequence. This secondary library is then amplified and screened as before.

While in some instances it may be appropriate to synthesize longer peptides to bind certain receptors, in other cases it may be desirable to provide peptides having two or more GPCR-binding sequences separated by spacer (e.g., linker) residues. For example, the binding sequences may be separated by spacers that allow the regions of the peptides to be presented to the receptor in different ways. The distance between binding regions may be as little as 1 residue, or at least 2-20 residues, or up to at least 100 residues. Preferred spacers are 3, 6, 9, 12, 15, or 18 residues in length. For probing large binding sites or tandem binding sites, the binding regions may be separated by a spacer of residues of up to 20 to 30 amino acids. The number of spacer residues when present will typically be at least 2 residues, and often will be less than 20 residues.

The oligonucleotide library may have binding sequences which are separated by spacers (e.g., linkers), and thus may be represented by the formula: (NNK)y−(abc)ₙ−(NNK)_{z} where N and K are as defined previously (note that S as defined previously may be substituted for K), and y+z is equal to about 5, 6, 7, 8, or more, a, b and c represent the same or different nucleotides comprising a codon encoding spacer amino acids, n is up to about 3, 6, 9, or 12 amino acids, or more. The spacer residues may be somewhat flexible, comprising oligo-glycine, or oligo-glycine-glycine-serine, for example, to provide the diversity domains of the library with the ability to interact with sites in a large binding site relatively unconstrained by attachment to the phage protein. Rigid spacers, such as, e.g., oligo-proline, may also be inserted separately or in combination with other spacers, including glycine spacers. It may be desired to have the GPCR-binding sequences close to one another and use a spacer to orient the binding sequences with respect to each other, such as by employing a turn between the two sequences, as might be provided by a spacer of the sequence glycine-proline-glycine, for example. To add stability to such a turn, it may be desirable or necessary to add cysteine residues at either or both ends of each variable region. The cysteine residues would then form disulfide bridges to hold the variable regions together in a loop, and in this fashion may also serve to mimic a cyclic peptide. Of course, those skilled in the art will appreciate that various other types of covalent linkages for cyclization may also be used.

Spacer residues as described above may also be situated on either or both ends of the GPCR-binding sequences. For instance, a cyclic peptide may be designed without an intervening spacer, by having a cysteine residue on both ends of the peptide. As described above, flexible spacers, e.g., oligo-glycine, may facilitate interaction of the peptide with the selected receptors. Alternatively, rigid spacers may allow the peptide to be presented as if on the end of a rigid arm, where the number of residues, e.g., proline residues, determines not only the length of the arm but also the direction for the arm in which the peptide is oriented. Hydrophilic spacers, made up of charged and/or uncharged hydrophilic amino acids, (e.g., Thr, His, Asn, Gln, Arg, Glu, Asp, Met, Lys, etc.), or hydrophobic spacers of hydrophobic amino acids (e.g., Phe, Leu, Ile, Gly, Val, Ala, etc.) may be used to present the peptides to receptor binding sites with a variety of local environments.

Notably, some peptides, because of their size and/or sequence, may cause severe defects in the infectivity of their carrier phage. This causes a loss of phage from the population during reinfection and amplification following each cycle of panning. To minimize problems associated with defective infectivity, DNA prepared from the eluted phage can be transformed into appropriate host cells, such as, e.g., E. coli, preferably by electroporation (see, e.g., Dower et al., Nucl. Acids Res. 16:6127-6145), or well known chemical means. The cells are cultivated for a period of time sufficient for marker expression, and selection is applied as typically done for DNA transformation. The colonies are amplified, and phage harvested for affinity enrichment in accordance with established methods. Phage identified in the affinity enrichment may be re-amplified by infection into the host cells. The successful transformants are selected by growth in an appropriate antibiotic(s), e.g., tetracycline or ampicillin. This may be done on solid or in liquid growth medium.

For growth on solid medium, the cells are grown at a high density (about 10⁸ to 10⁹ transformants per m²) on a large surface of, for example, L-agar containing the selective antibiotic to form essentially a confluent lawn. The cells and extruded phage are scraped from the surface and phage are prepared for the first round of panning (see, e.g., Parmley and Smith, 1988, Gene 73:305-318). For growth in liquid culture, cells may be grown in L-broth and antibiotic through about 10 or more doublings. The phage are harvested by standard procedures (see Sambrook et al., 1989, Molecular Cloning, 2^{nd} ed.). Growth in liquid culture may be more convenient because of the size of the libraries, while growth on solid media likely provides less chance of bias during the amplification process.

For affinity enrichment of desired clones, generally about 10³ to 10⁴ library equivalents (a library equivalent is one of each recombinant; 10⁴ equivalents of a library of 10⁹ members is 10⁹×10⁴=10¹³ phage), but typically at least 10² library equivalents, up to about 10⁵ to 10⁶, are incubated with a receptor (or portion thereof) to which the desired peptide is sought. The receptor is in one of several forms appropriate for affinity enrichment schemes. In one example the receptor is immobilized on a surface or particle, and the library of phage bearing peptides is then panned on the immobilized receptor generally according to procedures known in the art. In an alternate scheme, a receptor is attached to a recognizable ligand (which may be attached via a tether). A specific example of such a ligand is biotin. The receptor, so modified, is incubated with the library of phage and binding occurs with both reactants in solution. The resulting complexes are then bound to streptavidin (or avidin) through the biotin moiety. The streptavidin may be immobilized on a surface such as a plastic plate or on particles, in which case the complexes (phage/peptide/receptor/biotin/streptavidin) are physically retained; or the streptavidin may be labeled, with a fluorophor, for example, to tag the active phage/peptide for detection and/or isolation by sorting procedures, e.g., on a fluorescence-activated cell sorter.

Phage that associate with HGPRBMY3 or another GPC receptor via non-specific interactions are removed by washing. The degree and stringency of washing required will be determined for each receptor/peptide of interest. A certain degree of control can be exerted over the binding characteristics of the peptides recovered by adjusting the conditions of the binding incubation and the subsequent washing. The temperature, pH, ionic strength, divalent cation concentration, and the volume and duration of the washing will select for peptides within particular ranges of affinity for the receptor. Selection based on slow dissociation rate, which is usually predictive of high affinity, is the most practical route. This may be done either by continued incubation in the presence of a saturating amount of free ligand, or by increasing the volume, number, and length of the washes. In each case, the rebinding of dissociated peptide-phage is prevented, and with increasing time, peptide-phage of higher and higher affinity are recovered. Additional modifications of the binding and washing procedures may be applied to find peptides that bind receptors under special conditions. In a preferred embodiment of the invention, the disclosed cell-based panning protocol (see below) can be used to screen secondary libraries for peptides that bind to HGPRBMY3 or a related GPC receptor. Once a peptide sequence that imparts some affinity and specificity for the receptor molecule is known, the diversity around this binding motif may be embellished. For instance, variable peptide regions may be placed on one or both ends of the identified sequence. The known sequence may be identified from the literature, or may be derived from early rounds of panning in the context of this invention.

In particular aspects of the invention, secondary libraries may be produced using polynucleotides encoding the GPCR binders of the invention, or nucleic acids comprising these polynucleotides. For example, secondary libraries may be produced using the coding sequences (e.g., SEQ ID NO:47-SEQ ID NO:63, SEQ ID NO:67-SEQ ID NO:71, and SEQ ID NO:100-SEQ ID NO:104, or fragments or variants thereof) for GPCR-binding peptides, as described in detail herein. Peptides identified from secondary libraries are particularly useful for illustrating amino acid residues that are important for binding to HGPRBMY3 or other GPC receptors. Secondary library peptides can be used to determine consensus sequences (e.g., binding motifs) for GPCR binders, as well as the conserved residues and variable residues that comprise the consensus sequences. In certain cases, peptides identified from secondary libraries may have altered binding characteristics, for example, increased association rates or decreased dissociation rates, compared with the primary peptide sequence.

### Antibodies

The invention encompasses antibodies specific for HGPRBMY3 or GPCR-binding peptides (e.g., SEQ ID NO:17-SEQ ID NO:26, SEQ ID NO:35-SEQ ID NO:46, and SEQ ID NO:73-SEQ ID NO:77 or fragments, variants, or derivatives thereof) or polypeptides. Such antibodies can be generated using methods that have long been known and conventionally practiced in the art. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotype antibodies (including, e.g., anti-idiotype antibodies produced from the anti-binder antibodies of the invention), intracellularly made antibodies (i.e., intrabodies), and epitope-binding fragments of any of the above.

In one embodiment of the invention, the antibodies directed to GPCR-binding peptides or polypeptides can be utilized to generate anti-idiotypic antibodies that mimic the GPCR binders of the invention using techniques well known to those skilled in the art. See, e.g., Greenspan and Bona (1989) FASEB J. 7(5):437-444 and Nissinoff (1991) J. Immunol. 147(8):2429-2438. For example, antibodies directed to GPCR-binding peptides that competitively inhibit GPC receptor coupling or activation can be used to generate anti-idiotype antibodies. Preferably, peptides or peptide fragments that comprise one or more binding motifs (e.g., KIW, KVW, KLW, RVW, and TPHRVWXLP, wherein X is Q or N) are used to produce anti-idiotype antibodies. The anti-idiotype antibodies, or Fab fragments of such anti-idiotypes, can be used to mimic the peptides and, as a consequence, bind to and neutralize HGPRBMY3, or another GPC receptor, e.g., in therapeutic regimens. The neutralizing anti-idiotype antibodies or Fab fragments can thereby be used as antagonist or inhibitory agents, which can be formulated into pharmaceutical compositions, as described in detail herein.

The antibodies of the invention include immunoglobulin molecules and immunologically active portions or fragments of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class or subclass (e.g., IgG1, IgG2, IgG3, IgG4, IgA1 and IgA2) of immunoglobulin molecule. In a preferred embodiment, the immunoglobulin is an IgG1 isotype. In another preferred embodiment, the immunoglobulin is an IgG2 isotype. In another preferred embodiment, the immunoglobulin is an IgG4 isotype. Immunoglobulins may have both a heavy and a light chain. An array of IgG, IgE, IgM, IgD, IgA, and IgY heavy chains can be paired with a light chain of the kappa or lambda types. Most preferably, the antibodies of the present invention are human antigen-binding antibodies and antibody fragments and include, but are not limited to, Fab, Fab° F.(ab′) 2, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of the following: hinge region, and CH1, CH2, and CH3 domains. Also included in connection with the invention are antigen-binding fragments also comprising any combination of variable region(s) with a hinge region, and CH1, CH2, and CH3 domains.

The antibodies of the invention may be from any animal origin including birds and mammals. Preferably, the antibodies are of human, murine (e.g., mouse and rat), donkey, sheep, rabbit, goat, guinea pig, camel, horse, or chicken origin. As used herein, humanized antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example, in U.S. Pat. No. 5,939,598. The antibodies of the present invention can be monospecific, bispecific, trispecific, or of greater multispecificity. In one aspect, multispecific antibodies can be specific for different epitopes of a GPCR-binding peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof) or polypeptide, or can be specific for both a binder, and a heterologous epitope, such as a heterologous protein or solid support material. See, e.g., PCT publications WO 93/17715; WO 92/08802; WO 91/00360; WO 92/05793; Tutt et al. (1991) J. Immunol. 147:60-69; U.S. Pat. Nos. 4,474,893; 4,714,681; 4,925,648; 5,573,920; 5,601,819; and Kostelny et al. (1992) J. Immunol. 148:1547-1553.

Antibodies of the present invention can also be described or specified in terms of their binding affinity to a GPCR-binding peptide or polypeptide of the present invention. Preferred binding affinities include those with a dissociation constant or Kd of less than 5×10⁻² M, 1×10⁻² M, 5×10⁻³ M, 1×10⁻³ M, 5×10⁻⁴ M, or 1×10⁻⁴ M. More preferred binding affinities include those with a dissociation constant or Kd less than 5×10⁻⁵ M, 1×10⁻⁵ M, 5×10⁻⁶ M, 1×10′ M, 5×10⁻⁷ M, 1×10⁻⁷ M, 5×10-8 M, or 1×10⁻⁸ M. Even more preferred antibody binding affinities include those with a dissociation constant or Kd of less than 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻³ M, 5×10⁻¹⁴ M, 1×10⁻¹⁴ M, 5×10⁻¹⁵ M, or 1×10⁻¹⁵ M. The present invention also provides antibodies that competitively inhibit the binding of an antibody to an epitope of the invention as determined by any method known in the art for determining competitive binding, for example, the immunoassays as described herein. In preferred embodiments, the antibody competitively inhibits binding to an epitope by at least 95%, at least 90%, at least 85%, at least 80%, at least 75%, at least 70%, at least 60%, or at least 50%.

For the production of antibodies, various hosts including goats, rabbits, sheep, rats, mice, humans, and others, can be immunized by injection with GPCR-binding peptide or polypeptide that has immunogenic properties. Depending on the host species, various adjuvants may be used to increase the immunological response. Non-limiting examples of suitable adjuvants include Freund's (incomplete), mineral gels such as aluminum hydroxide or silica, and surface-active substances such as lysolecithin, pluronic polyols, polyanions, peptides, oil emulsions, KLH, and dinitrophenol. Adjuvants typically used in humans include BCG (bacilli Calmette Guerin) and Corynebacterium parvumn. Preferably, the peptides, fragments, or oligopeptides (i.e., immunogens) used to induce antibodies to GPCR binders comprise at least 5 contiguous amino acids, and more preferably, at least 7-10 amino acids of SEQ ID NO:17-SEQ ID NO:26, SEQ ID NO:35-SEQ ID NO:46, SEQ ID NO:73-SEQ ID NO:77. It is also preferable that the immunogens are identical to a portion of the original amino acid sequence. The immunogens may also contain the entire amino acid sequence of a small, naturally occurring molecule. The peptides, fragments or oligopeptides may comprise a single epitope or antigenic determinant or multiple epitopes. Short stretches of amino acids may be fused with those of another protein, such as KLH, and antibodies are produced against the chimeric molecule.

Monoclonal antibodies to GPCR-binding peptides, polypeptides, or immunogenic fragments thereof, may be prepared using any technique which provides for the production of antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-.42; R. J. Cote et al., 1983, Proc. Natl. Acad. Sci. USA, 80:2026-2030; and S. P. Cole et al., 1984, Mol. Cell Biol., 62:109-120). The production of monoclonal antibodies is well known and routinely used in the art.

In addition, established methods may be used to produce chimeric antibodies. Such methods involve the splicing of mouse antibody genes to human antibody genes to obtain a molecule with appropriate antigen specificity and biological activity (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques for producing single chain antibodies may be adapted, using methods known in the art, to produce binder-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci USA, 86:3833-3837 and G. Winter et al., 1991, Nature, 349:293-299).

Antibody fragments, which contain specific binding sites for a GPCR-binding peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof) or polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., 1989, Science, 254.1275-1281). Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between a GPCR-binding peptide or polypeptide and its specific antibody. A two-site, monoclonal-based immunoassay utilizing monoclonal antibodies reactive with two non-interfering GPCR binder epitopes is preferred, but a competitive binding assay may also be employed (Maddox, supra).

The GPCR-binding peptides (e.g., SEQ ID NO:17-SEQ ID NO:26, SEQ ID NO:35-SEQ ID NO:46, and SEQ ID NO:73-SEQ ID NO:77, or fragments, variants, or derivatives thereof) or polypeptides of the invention may also be synthesized as multiple antigen peptides (MAPs), first described by J. P. Tam et al. (1995) Biomed. Pept, Proteins, Nucleic Acids, 199, 1(3):123-32; and Calvo, et al. (1993) J. Immunol., 150(4):1403-12), which are hereby incorporated herein by reference in their entirety. MAPs contain multiple copies of a specific peptide attached to a non-immunogenic lysine core. MAP peptides usually contain four or eight copies of the peptide, which are often referred to as MAP4 or MAP8 peptides. By way of non-limiting example, MAPs can be synthesized onto a lysine core matrix attached to a polyethylene glycol-polystyrene (PEG-PS) support. The peptide of interest is synthesized onto the lysine residues using 9-fluorenylmethoxycarbonyl (Fmoc) chemistry. For example, Applied Biosystems (Foster City, Calif.) offers commercially available MAP resins, such as, for example, the Fmoc Resin 4 Branch and the Fmoc Resin 8 Branch, which can be used to synthesize MAPs. Cleavage of MAPs from the resin is performed with standard trifloroacetic acid (TFA)-based cocktails known in the art. Purification of MAPs, except for desalting, is not generally necessary. MAP peptides can be used in immunizing vaccines which elicit antibodies that recognize both the MAP and the native protein from which the peptide was derived.

The GPCR-binding peptides (e.g., SEQ ID NO:17-SEQ ID NO:26, SEQ ID NO:35-SEQ ID NO:46, and SEQ ID NO:73-SEQ ID NO:77 or fragments, variants, or derivatives thereof) or polypeptides of the invention can also be incorporated into a coat protein of a virus, which can then be used as an immunogen or a vaccine with which to immunize animals, including humans, in order stimulate the production of anti-epitope antibodies. For example, the V3 loop of the gp120 glycoprotein of the human immunodeficiency virus type 1 (HIV-1) has been engineered to be expressed on the surface of rhinovirus. Immunization with rhinovirus displaying the V3 loop peptide yielded apparently effective mimics of the HIV-1 immunogens (as measured by their ability to be neutralized by anti-HIV-1 antibodies as well as by their ability to elicit the production of antibodies capable of neutralizing HIV-1 in cell culture). This techniques of using engineered viral particles as immunogens is described in more detail in Smith et al. (1997) Behring Inst Mitt Feb, 98:229-39; Smith et al. (1998) J. Virol. 72:651-659; and Zhang et al. (1999) Biol. Chem. 380:365-74), which are hereby incorporated by reference herein in their entireties.

For the production of antibodies in vivo, host animals, such as rabbits, rats, mice, sheep, or goats, are immunized with either free or carrier-coupled peptides or MAP peptides, for example, by intraperitoneal and/or intradermal injection. Injection material is typically an emulsion containing about 100 μg of peptide or carrier protein and Freund's adjuvant, or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal can be increased by selection of anti-peptide antibodies, e.g., by adsorption of the peptide onto a solid support and elution of the selected antibodies according to methods well known in the art.

In one aspect of the invention, XenoMouse Technology Antibodies may be prepared in accordance with the known techniques. Such techniques involve the utilization of a transgenic mouse that has a substantial portion of the human antibody producing genome inserted, but that is rendered deficient in the production of endogenous murine antibodies (e.g., XenoMouse strains available from Abgenix Inc., Fremont, Calif.). These mice are capable of producing human immunoglobulin molecules and antibodies and are virtually deficient in the production of murine immunoglobulin molecules and antibodies. Technologies utilized for achieving the same are disclosed in the patents, applications, and references disclosed herein.

As one having skill in the art will appreciate, and as discussed above, the GPCR-binding peptides (e.g., SEQ ID NO:17-SEQ ID NO:26, SEQ ID NO:35-SEQ ID NO:46, and SEQ ID NO:73-SEQ ID NO:77 or fragments, variants, or derivatives thereof) or polypeptides of the present invention, which comprise an immunogenic or antigenic epitope, can be fused to other polypeptide sequences. Such fusion proteins may facilitate purification and may increase half-life in vivo. For example, the polypeptides of the present invention can be fused with the constant domain of immunoglobulins (IgA, IgE, IgG, IgD, or IgM), or portions thereof, e.g., CH1, CH2, CH3, or any combination thereof, and portions thereof, or with albumin (including, but not limited to, recombinant human albumin, or fragments or variants thereof (see, e.g., U.S. Pat. No. 5,876,969; EP Patent No. 0 413 622; and U.S. Pat. No. 5,766,883, incorporated by reference in their entirety herein), This has been shown for chimeric proteins containing the first two domains of the human CD4-polypeptide and various domains of the constant regions of the heavy or light chains of mammalian immunoglobulins. See, e.g., Traunecker et al. (1988) Nature 331:84-86). Enhanced delivery of an antigen across the epithelial barrier to the immune system has been demonstrated for antigens (e.g., insulin) conjugated to an FcRn binding partner, such as IgG or Fc fragments (see, e.g., PCT Publications WO 96/22024 and WO 99/04813). IgG fusion proteins that have a disulfide-linked dimeric structure due to the IgG portion disulfide bonds have also been found to be more efficient in binding and neutralizing other molecules than are monomeric polypeptides, or fragments thereof, alone. See, e.g., Fountoulakis et al. (1995) J. Biochem. 270:3958-3964.

In another aspect of the invention, established methods can be used to prepare intrabodies. Intrabodies are antibodies, often scFvs, that are expressed from a recombinant nucleic acid molecule and are engineered to be retained intracellularly (e.g., retained in the cytoplasm, endoplasmic reticulum, or periplasm of the host cells). Intrabodies (e.g., anti-idiotype intrabodies) can be used, for example, to ablate or inhibit the function of a GPC receptor to which the intrabody binds. The expression of intrabodies can also be regulated through the use of inducible promoters in the nucleic acid expression vector comprising nucleic acid encoding the intrabody. Intrabodies of the invention can be produced using methods known in the art, such as those disclosed and reviewed in Chen et al. (1994) Hum. Gene Ther. 5:595-601; W. A. Marasco (1997) Gene Ther. 4:11-15; Rondon and Marasco (1997) Annu. Rev. Microbiol. 51:257-283; Proba et al. (1998) J. Mol. Biol. 275:245-253; Cohen et al. (1998) Oncogene, 17:2445-2456; Ohage and Steipe (1999) J. Mol. Biol. 291:1119-1128; Ohage et al. (1999) J. Mol. Biol. 291:1129-1134; Wirtz and Steipe (1999) Protein Sci. 8:2245-2250; Zhu et al. (1999) J. Immunol. Methods, 231:207-222.

The present invention further encompasses antibodies or fragments thereof conjugated to a diagnostic or therapeutic agent. The antibodies can be used diagnostically to, for example, monitor the development or progression of a tumor as part of a clinical testing procedure, for example, to determine the efficacy of a given treatment regimen. Detection can be facilitated by coupling the antibody to a detectable substance. Nonlimiting examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody (or fragment thereof) or indirectly, through an intermediate (such as, for example, a linker known in the art) using techniques known in the art. (See, for example, U.S. Pat. No. 4,741,900 for metal ions that can be conjugated to antibodies for use as diagnostics according to the present invention).

Nonlimiting examples of suitable enzymes include horseradish peroxidase, alkaline phosphatase, beta-galactosidase, or acetylcholinesterase; nonlimiting examples of suitable prosthetic group complexes include streptavidin/biotin and avidin/biotin; nonlimiting examples of suitable fluorescent materials include umbelliferone, fluorescein, fluorescein isothiocyanate, rhodamine, dichlorotriazinylamine fluorescein, dansyl chloride or phycoerythrin; a nonlimiting example of a luminescent material includes luminol; nonlimiting examples of bioluminescent materials include luciferase, luciferin, and aequorin; and nonlimiting examples of suitable radioactive material include iodine (¹²⁵I, ¹³¹I), carbon (¹⁴C), sulfur (³⁵S), tritium (³H), indium (¹¹¹In and other radioactive isotopes of inidium), technetium (⁹⁹Tc, ^{99m}Tc), thallium (20′Ti), gallium (⁶⁸Ga, ⁶⁷Ga), palladium (¹⁰³Pd), molybdenum (⁹⁹Mo), xenon (¹³³Xe), fluorine (¹⁹F),¹⁵³Sm, ¹⁷⁷Lu, Gd, radioactive Pm, radioactive La, radioactive Yb, ¹⁶⁶Ho, ⁹⁰Y, radioactive Sc, radioactive Re, radioactive Re, ¹⁴²Pr, ¹⁰⁵Rh, and ⁹⁷Ru.

In specific embodiments, the antibodies of the invention are attached to macrocyclic chelators useful for conjugating radiometal ions, including, but not limited to, ¹¹¹In, ¹⁷⁷Lu, ⁹⁰Y, ¹⁶⁶Ho, and ¹⁵³Sm, to polypeptides. In a preferred embodiment, the radiometal ion associated with the macrocyclic chelators attached to the B7-related polypeptides of the invention is ¹¹¹In. In another preferred embodiment, the radiometal ion associated with the macrocyclic chelator attached to the antibodies of the invention is ⁹⁰Y. In specific embodiments, the macrocyclic chelator is 1, 4, 7, 10-tetraazacyclododecane-N, N′, N″, N′″-tetraacetic acid (DOTA). In other specific embodiments, the DOTA is attached to the antibodies of the invention via a linker molecule.

Examples of linker molecules useful for conjugating DOTA to a polypeptide are commonly known in the art. (See, for example, DeNardo et al. (1998) Clin. Cancer Res. 4(10):2483-90; Peterson et al. (1999) Bioconjug. Chem. 10(4):553-557; and Zimmerman et al. (1999) Nucl. Med. Biol. 26(8): 943-950, which are hereby incorporated by reference in their entirety. In addition, U.S. Pat. Nos. 5,652,361 and 5,756,065, which disclose chelating agents that can be conjugated to antibodies and methods for making and using them, are hereby incorporated by reference in their entireties. Though U.S. Pat. Nos. 5,652,361 and 5,756,065 focus on conjugating chelating agents to antibodies, one skilled in the art can readily adapt the methods disclosed therein in order to conjugate chelating agents to other polypeptides. Antibodies can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating therapeutic moieties to antibodies are well known, see, e.g., Arnon et al. (1985) “Monoclonal Antibodies For Immunotargeting Of Drugs In Cancer Therapy”, Monoclonal Antibodies And Cancer Therapy, Reisfeld et al., eds., Alan R. Liss, Inc., pp. 243-56; Hellstrom et al. (1987) “Antibodies For Drug Delivery”, Controlled Drug Delivery, 2nd Ed., Robinson et al. (eds.), Marcel Deldcer, Inc., pp. 623-53; Thorpe, (1985) “Antibody Carriers Of Cytotoxic Agents In Cancer Therapy: A Review”, Monoclonal Antibodies '84: Biological And Clinical Applications, Pinchera et al., eds., pp. 475-506; Baldwin et al., eds., (1985) “Analysis, Results, And Future Prospective Of The Therapeutic Use Of Radiolabeled Antibody In Cancer Therapy”, Monoclonal Antibodies For Cancer Detection And Therapy, Academic Press, pp. 303-316; and Thorpe et al. (1982) Immunol. Rev. 62:119-158. Alternatively, an antibody can be conjugated to a second antibody to form an antibody heteroconjugate, e.g., as described in U.S. Pat. No. 4,676,980 to Segal, which is incorporated herein by reference in its entirety. An antibody, i.e., an antibody specific for a B7-related polypeptide of this invention, with or without a therapeutic moiety conjugated to it, and administered alone or in combination with cytotoxic factor(s) and/or cytokine(s), can be used as a therapeutic.

Antibodies according to this invention can be assayed for immunospecific binding by any method known in the art. The immunoassays which can be used include, but are not limited to, competitive and non-competitive assay systems using techniques such as BIAcore analysis, FACS (Fluorescence Activated Cell Sorter) analysis, immunofluorescence, immunocytochemistry, Western blots, radioimmunoassays, ELISA (enzyme linked immunosorbent assays), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, to name but a few. Such assays are routine and well known and practiced in the art (see, e.g., Ausubel et al., eds., (1994) Current Protocols in Molecular Biology, Vol. 1, John Wiley & Sons, Inc., New York, which is incorporated by reference herein in its entirety). Nonlimiting, exemplary immunoassays are described briefly below.

The present invention also encompasses the creation of synthetic antibodies directed against the peptides and polypeptides of the present invention. One example of synthetic antibodies is described in Radrizzani, M., et al., Medicina, (Aires), 59(6):753-8, (1999)). A new class of synthetic antibodies has been described and is referred to as molecularly imprinted polymers (MIPs) (Semorex, Inc.). Molecularly imprinted polymers (MIPs) are capable of mimicking the function of antibodies with fewer stability constraints. Such polymers provide high sensitivity and selectivity, while maintaining excellent thermal and mechanical stability. MIPs have the ability to bind to small molecules and to target molecules such as organics and proteins with equal or greater potency than that of natural antibodies. These so-called super MIPs have higher affinities for their target and thus require lower concentrations for efficacious binding.

MIPs that mimic the structure of the peptides or polypeptides of the present invention may be useful in screening for compounds that bind to these peptides or polypeptides. Such MIPs would serve as binder mimetics, and imitate the structure and function of the binders of the invention. The use of MIPs has previously been demonstrated (Ye, L., Yu, Y., Mosbach, K., Analyst., 126(6):760-5, (2001); Dickert, F. L., Hayden, O., Halikias, K. P., Analyst., 126(6):766-71, (2001)). A peptide or polypeptide of the invention may be mimicked in its entirety (e.g., as the entire sequence), or mimicked as a series of short peptides corresponding to the sequence (Rachkov, A., Minoura, N, Biochm, Biophys, Acta., 1544(1-2):255-66, (2001)). Such MIPs may be employed in any one or more of the screening methods described herein.

MIPs have also been shown to be useful in identifying a molecule of interest (Cheng, Z., Wang, E., Yang, X., Biosens, Bioelectron., 16(3):179-85, (2001); Jenkins, A. L., Yin, R., Jensen, J. L., Analyst., 126(6):798-802, (2001); Jenkins, A. L., Yin, R., Jensen, J. L., Analyst., 126(6):798-802, (2001)). For example, a MIP designed using a peptide of the present invention may be used in assays designed to identify, and potentially quantitate, the level of the peptide in a sample. The same MIP may be used as a substitute for any component described in the assays, or kits, provided herein (e.g., ELISA, etc.).

During synthesis, MIPs are imprinted so as to have complementary size, shape, charge and functional groups of the selected target by using the target molecule itself (such as a peptide, polypeptide, antibody, etc.), or a substance having a very similar structure, e.g., its print or template. MIPs can be derivatized with the same reagents used for antibodies. For example, super MIPs conjugated to fluorescent molecules can be coated onto beads or wells for use in highly sensitive separations or assays, or for use in high throughput screening of proteins. A number of methods may be employed to create MIPs to a specific receptor, ligand, polypeptide, peptide, or organic molecule. Several preferred methods are described by Esteban et al. in J. Anal, Chem., 370(7):795-802, (2001), which is hereby incorporated herein by reference in its entirety in addition to any references cited therein. Additional methods are known in the art and are encompassed by the present invention, such as for example, Hart, B, R., Shea, K, J. J. Am. Chem, Soc., 123(9):2072-3, (2001); and Quaglia, M., Chenon, K., Hall, A, J., De, Lorenzi, E., Sellergren, B, J. Am. Chem. Soc., 123(10):2146-54, (2001); which are hereby incorporated by reference in their entirety herein.

### Diagnostics

The present invention also encompasses methods of using the disclosed antibodies, anti-idiotype antibodies, peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), as well as oligopeptides, and polypeptides comprising these peptides, as diagnostic reagents. A variety of protocols exist for detecting and measuring the expression of HGPRBMY3 (e.g., SEQ ID NO:2) or a related GPC receptor (e.g., SEQ ID NO:110-SEQ ID NO:112) using the anti-idiotype antibodies of the invention. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on the HGPRBMY3 polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216).

In various aspects, anti-idiotype antibodies which specifically bind to HGPRBMY3 or a related GPC receptor (e.g., SEQ ID NO:2, SEQ ID NO:110-SEQ ID NO:112, or a fragment thereof) may be used for the diagnosis of conditions or diseases characterized by mutation, truncation, or altered expression of the polypeptide, or in assays to monitor patients being treated with HGPRBMY3, HGPRBMY11, HGPRBMY23, P2Y10, or its agonists, antagonists, or inhibitors. The antibodies useful for diagnostic purposes may be prepared in the same manner as those for use in therapeutic methods. Diagnostic assays for GPC receptors include methods, which utilize the anti-idiotype antibody and a label to detect the protein in human body fluids or extracts of cells or tissues. The antibodies may be used with or without modification, and may be labeled by joining them, either covalently or non-covalently, with a reporter molecule. A wide variety of reporter molecules, which are known in the art, may be used, several of which are described above.

In accordance with the invention, the disclosed peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), and polypeptides comprising these peptides, can also be used in diagnostic assays. For example, GPCR-binding peptides or polypeptides can be used to detect a mutated or truncated form of a GPC receptor, e.g., HGPRBMY3, associated with a disease or susceptibility to a disease. In one aspect of the invention, certain binders may specifically recognize only a mutated or truncated form of HGPRBMY3 or another GPC receptor. Alternatively, certain peptides or polypeptides of the invention may recognize only an unaltered GPC receptor sequence (e.g., SEQ ID NO:2, SEQ ID NO:110-SEQ ID NO:112, or a fragment thereof). These binders can be used side-by-side with binding controls to diagnose or monitor HGPRBMY3- or other GPCR-related disorders. As other examples, GPCR-binding peptides or polypeptides can be used to detect the under-expression, over-expression, or altered expression of a GPC receptor, which results in onset of a disease or susceptibility to a disease.

For diagnosis, a GPC receptor may be present in a biological sample, such as from blood, urine, saliva, tissue biopsy or autopsy material. Deletions and insertions in the GPC receptor can be confirmed by a change in size (e.g., electrophoretic migration) of the gene product in comparison to the normal product. The diagnostic assays offer a process for diagnosing or determining a susceptibility to infections such as bacterial, fungal, protozoan and viral infections, particularly infections caused by HIV-1 or HIV-2 through detection of altered sequence or expression of HGPRBMY3 or another GPC receptor by the methods described. The invention also provides assays for determining for diagnosing the presence of or susceptibility to the medical conditions, diseases, or disorders as described in detail herein.

In one embodiment, the diagnostic method comprises: a) incubating an HGPRBMY3-binding peptide with a biological sample under conditions to allow the peptide to associate with an HGPRBMY3 polypeptide in the sample; and b) measuring levels of peptide-polypeptide complex formed in (a), wherein an alteration in these levels compared to standard levels indicates diagnosis of the medical condition. In another embodiment, the diagnostic method comprises: a) incubating an HGPRBMY3-binding anti-idiotype antibody with a biological sample under conditions to allow the antibody to associate with an HGPRBMY3 polypeptide in the sample; and b) measuring levels of antibody-polypeptide complex formed in (a), wherein an alteration in these levels compared to standard levels indicates diagnosis of the medical condition.

The present invention further relates to a kit for monitoring or diagnosing a disease or condition, particularly those identified herein, which comprises: (a) an HGPRBMY3-binding peptide or polypeptide, preferably comprising an amino acid sequence of any one of SEQ ID NO:17-SEQ ID NO:26, SEQ ID NO:35-SEQ ID NO:46, or a fragment thereof; or (b) an anti-idiotype antibody that recognizes an HGPRBMY3 polypeptide (e.g., SEQ ID NO:2, or a fragment thereof), preferably derived from an antibody to an amino acid sequence of any one of SEQ ID NO:17-SEQ ID NO:26 or SEQ ID NO:35-SEQ ID NO:46. It will be appreciated that in any such kit, (a) or (b) may comprise a substantial component. The kit components may be pre-labeled, or may be supplied with labeling reagents. Optionally, a kit may also include buffers, standards, controls, and/or directions for use.

In a particular aspect, a GPCR-bindng anti-idiotype antibody, peptide, or polypeptide may be useful in assays that detect or monitor activation or induction of various neoplasms or cancers, particularly those mentioned herein. The GPCR-bindng anti-idiotype antibody, peptide or polypeptide may be labeled by standard methods, and added to a fluid or tissue sample from a patient, under conditions suitable for the formation of antibody-GPCR, peptide-GPCR, or polypeptide-GPCR complexes. After a suitable incubation period, the sample is washed and the signal is quantified and compared with a standard value. If the amount of signal in the biopsied or extracted sample is significantly altered from that of a comparable control sample, this indicates the presence of the associated disease. Such assays may also be used to evaluate the efficacy of a particular therapeutic treatment regimen in animal studies, in clinical trials, or in monitoring the treatment of an individual patient.

To provide a basis for the diagnosis of disease associated with expression of a GPC receptor, a normal or standard profile for expression is established. This may be accomplished by combining a biological sample taken from normal subjects, either animal or human, with a sequence, or a fragment thereof, which contains the GPC receptor, under conditions suitable for binding to a peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof), a polypeptide comprising this peptide, or an anti-idiotype antibody that binds to a GPC receptor. Standard binding may be quantified by comparing the values obtained from normal subjects with those from an experiment where a known amount of a substantially purified HGPRBMY3 polypeptide (e.g., SEQ ID NO:2) or other GPC receptor (e.g., SEQ ID NO:110-SEQ ID NO:112) is used. The amount of standard binding (i.e., complex formation) may be quantified by various methods; photometric means are preferred. Standard values obtained from normal samples may be compared with values obtained from samples from patients who are symptomatic or have a family history of disease. Deviation between standard and subject (patient) values is used to establish the presence of disease.

Once disease is established and a treatment protocol is initiated, binding assays may be repeated on a regular basis to evaluate whether the level of expression in the patient begins to approximate that which is observed in a normal individual. The results obtained from successive assays may be used to show the efficacy of treatment over a period ranging from several days to months. With respect to cancer, the presence of an abnormal amount of HGPRBMY3 polypeptide (e.g., SEQ ID NO:2) in blood or biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer. The speed of quantifying multiple samples may be accelerated by running the assay in an ELISA format where the binder is presented in various dilutions and a spectrophotometric or colorimetric response gives rapid quantification.

### Therapeutics

The present invention encompasses methods of using the disclosed antibodies, anti-idiotype antibodies, peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), polypeptides comprising these peptides, and other modulators of the invention, as therapeutic agents. Also useful as therapeutics are the polynucleotides of the invention (e.g., SEQ ID NO:47-SEQ ID NO:63, SEQ ID NO:67-SEQ ID NO:71, and SEQ ID NO:100-SEQ ID NO:104, and fragments, variants, and derivatives thereof), vectors comprising these polynucleotides, and host cells comprising these vectors. As indicated previously, the HGPRBMY3 polypeptide may play a role in cell-cycle regulation, and/or cell signaling, and may be involved in immune, testicular, colonic, breast, and ovarian disorders, and/or neoplastic, proliferative, neurological, and immunological disorders.

For proliferative disorders, antagonist or inhibitory agents of HGPRBMY3 (e.g., SEQ ID NO:2) may be administered to an individual to prevent, treat, or ameliorate these disorders. Specific agents can include one or more of the peptides, polypeptides, anti-idiotype antibodies, and other modulators, as well as one or more of the polynucleotides, vectors, and host cells of the invention. Non-limiting examples of proliferative disorders include adenocarcinoma, leukemia, lymphoma, myeloma, melanoma, squamous cell carcinoma, sarcoma (e.g., fibrosarcoma), and teratocarcinoma, and particularly, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, as well as other disorders listed herein. In a related aspect, an anti-idiotype antibody which specifically binds to HGPRBMY3 may be used directly as an antagonist or indirectly as a targeting or delivery mechanism for bringing a pharmaceutical agent to cells or tissue which express the HGPRBMY3 polypeptide.

In one embodiment, the therapeutic method of the invention comprises: administering to a subject with cancer a pharmaceutical composition comprising an HGPRBMY3-binding peptide or combination of peptides and a physiologically acceptable carrier, excipient, or diluent, in an amount sufficient to treat the cancer. In another embodiment, the method comprises: administering to a subject with cancer a pharmaceutical composition comprising an HGPRBMY3-binding anti-idiotype antibody or combination of antibodies and a physiologically acceptable carrier, excipient, or diluent, in an amount sufficient to treat the cancer. In a further embodiment, the method comprises: administering to a subject with cancer a pharmaceutical composition comprising a vector encoding an HGPRBMY3-binding peptide or combination of peptides and a physiologically acceptable carrier, excipient, or diluent, in an amount sufficient to treat the cancer. In yet another embodiment, the method comprises: administering to a subject with cancer a pharmaceutical composition comprising a host cell expressing an HGPRBMY3-binding peptide or combination of peptides and a physiologically acceptable carrier, excipient, or diluent, in an amount sufficient to treat the cancer.

In various aspects of the present invention, a modulatory agent of an HGPRBMY3 polypeptide (e.g., SEQ ID NO:2) may be administered to an individual to prevent, treat, or ameliorate a neurological or, preferably, an immunological disorder. Specific agents can include one or more of the peptides, polypeptides, antibodies, anti-idiotype antibodies, vectors, and other modulators of the invention. Non-limiting examples of immunological disorders include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, leukemia, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections, and trauma, as well as other disorders described herein. In preferred embodiments, the peptides, polypeptides, anti-idiotype antibodies, and vectors of the invention are useful for modulating intracellular Ca²⁺ levels, modulating Ca²⁺ sensitive signaling pathways, and modulating NFAT element associated signaling pathways.

In another embodiment, the peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), polypeptides, antibodies, anti-idiotype antibodies, vectors, host cells, and other modulators of the present invention can be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors which will express nucleic acid sequences (e.g., SEQ ID NO:47-SEQ ID NO:63, SEQ ID NO:67-SEQ ID NO:71, and SEQ ID NO:100-SEQ ID NO:104, or fragments, variants, or derivatives thereof) encoding a GPCR-binding peptide or polypeptide. These techniques are described both in J. Sambrook et al., supra and in F. M. Ausubel et al., supra. GPCR-binding peptides and polypeptides used in treatment can also be generated endogenously in the subject, in treatment modalities often referred to as gene therapy. Thus for example, cells from a subject may be engineered with a polynucleotide, such as DNA or RNA, to encode a peptide or polypeptide ex vivo, and for example, by the use of a retroviral plasmid vector. The cells can then be introduced into the subject. Many methods for introducing vectors into cells or tissues are available and are equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise one or more peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), polypeptides, antibodies, anti-idiotype antibodies, vectors, host cells, or other modulators of the invention. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers. Any of the therapeutic methods described herein may be applied to any individual in need of such therapy, including, for example, mammals such as dogs, cats, cows, horses, rabbits, monkeys, and most preferably, humans.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means. Because the modulators of the invention (e.g., antibodies, peptides, polypeptides, etc.) may be broken down in the stomach, these agents are preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials, and may be stored in a freeze-dried condition requiring only the addition of the sterile liquid carrier immediately prior to use. The formulation may also include adjuvant systems for enhancing the immunogenicity of the formulation, such as oil-in-water systems and other systems known in the art. The dosage will depend on the specific activity of the antibody, peptide, or polypeptide, and can be readily determined by routine experimentation.

In addition to the active ingredients (e.g., the peptides, polypeptides, antibodies, anti-idiotype antibodies, vectors, or other modulators of the invention), the pharmaceutical compositions may contain suitable pharmaceutically acceptable carriers or excipients comprising auxiliaries which facilitate processing of the active compounds into preparations which can be used pharmaceutically. Further details on techniques for formulation and administration are provided in the latest edition of Remington's Pharmaceutical Sciences (Mack Publishing Co., Easton, Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate. Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers. Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents that increase the solubility of the compounds to allow for the preparation of highly concentrated solutions. For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of a GPCR binder, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, a peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof), polypeptide, antibody, anti-idiotype antibody, vector, host cell, or other modulator of the invention which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, ED₅₀/LD₅₀. Pharmaceutical compositions, which exhibit large therapeutic indices, are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The exact dosage will be determined by the practitioner, who will consider the factors related to the individual requiring treatment. Dosage and administration are adjusted to provide sufficient levels of the active moiety or to maintain the desired effect. Factors, which may be taken into account, include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art. Normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

Agents which are identified according to the methods provided herein, and which modulate or regulate the biological activity or physiology of HGPRBMY3 (e.g., SEQ ID NO:2) or another GPC receptor (e.g., SEQ ID NO:110-SEQ ID NO:112) are encompassed by this invention. It is contemplated that such modulatory agents may be employed in treatment and therapeutic methods for treating a condition that is mediated by the novel HGPRBMY3 polypeptides by administering to an individual in need of such treatment a therapeutically effective amount of the agent identified by the methods described herein. In addition, the present invention provides methods for treating an individual in need of such treatment for a disease, disorder, or condition that is mediated by an HGPRBMY3 polypeptide of the invention, comprising administering to the individual a therapeutically effective amount of the HGPRBMY3-modulating agent identified by a method provided herein.

Peptides, polypeptides, anti-idiotype antibodies and/or other modulators of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. The modulatory agent(s) may be administered prior to an extraterrestrial event, during an extraterrestrial event, or both prior and during the event. This use may result in a number of beneficial changes in the recipient, such as, for example, any one of the following, non-limiting, effects: an increased level of hematopoietic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoietic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid the recipient by decreasing their basal daily energy requirements; (ii) effectively lower the level of oxidative and/or metabolic stress in recipient (i.e., to enable recipient to cope with increased extraterrestrial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Peptides, polypeptides, antibodies, anti-idiotype antibodies, and/or other modulators of the present invention may also be used to increase the efficacy of a pharmaceutical composition, either directly or indirectly. The modulatory agent(s) may be administered in simultaneous conjunction with said pharmaceutical, or separately through either the same or different route of administration (e.g., intravenous for the polynucleotide or polypeptide of the present invention, and orally for the pharmaceutical, among others described herein.).

### Screening Assays

### Test Agents

The invention further encompasses agents that modulate and/or bind to GPC receptors that are identified using the disclosed antibodies, anti-idiotype antibodies, peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), disclosed herein. Essentially any test agent can be employed as a potential binder, modulator, or ligand in the assays according to the present invention. Test agents can include any small chemical agent, or biological component (e.g., protein, sugar, nucleic acid, or lipid). Test agents will typically be small chemical molecules and peptides. Generally, the agents used as potential modulators can be dissolved in aqueous or organic (e.g., DMSO-based) solutions. There are many suppliers of chemical agents, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, agents may be synthesized by methods known in the art.

The preferred assays of the invention are designed to screen large chemical libraries by automating the assay steps and providing agents from any convenient source. Assays are typically run in parallel, for example, in microtiter formats on microtiter plates in robotic assays. High-throughput screening methodologies are particularly envisioned for the detection of modulators and/or binders of the GPC receptors described herein. Such high-throughput screening methods typically involve providing a combinatorial chemical or peptide library containing a large number of potential therapeutic agents (e.g., ligand or modulator agents). Such combinatorial chemical libraries or ligand libraries are then screened in one or more assays to identify those library members (e.g., particular chemical species or subclasses) that display a desired characteristic activity. The agents so identified can serve as conventional lead agents, or can themselves be used as potential or actual therapeutics.

A combinatorial chemical library is a collection of diverse chemical agents generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given agent size (i.e., the number of amino acids in a polypeptide or peptide compound). Millions of chemical agents can be synthesized through such combinatorial mixing of chemical building blocks. The preparation and screening of combinatorial chemical libraries is well known to those having skill in the pertinent art. Combinatorial libraries include, but are not limited to, peptide libraries (e.g. U.S. Pat. No. 5,010,175; Furka, 1991, Int. J. Pept. Prot. Res., 37:487-493; and Houghton et al., 1991, Nature, 354:84-88).

In accordance with the invention, many chemical diversity libraries can be used. Nonlimiting examples include libraries comprising peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for preparing combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are available from commercial sources (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

To purify a GPC receptor (e.g., SEQ ID NO:2, SEQ ID NO:110-SEQ ID NO:112, or fragment thereof) in order to measure a biological binding or ligand binding activity, the source may be a whole cell lysate that can be prepared by successive freeze-thaw cycles (e.g., one to three) in the presence of standard protease inhibitors. The GPC receptor may be partially or completely purified by standard protein purification methods, e.g., affinity chromatography using specific antibody described infra, or by ligands specific for an epitope tag engineered into the recombinant GPC receptor molecule, also as described herein. Binding activity can then be measured as described.

### Binding Assays

Also encompassed are methods of using the disclosed antibodies, anti-idiotype antibodies, peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), and polypeptides to screen for other agents that bind to GPC receptors, such as HGPRBMY3 or related receptors (e.g., SEQ ID NO:2, SEQ ID NO:110-SEQ ID NO:112, or fragments thereof). The anti-idiotype antibodies, peptides, or polypeptides employed in such screening may be pre-incubated with a GPC receptor, or added as competitors to test agents. For screening, the anti-idiotype antibodies, peptides, or polypeptides may be free in solution, affixed to a solid support, borne on a cell surface, or located intracellularly. The formation of binding complexes between a GPC receptor, or portion thereof, and the agents being tested may be measured utilizing techniques commonly practiced in the art. Alternatively, a binding assay can be conducted in a liquid phase, the reaction products separated from unreacted components, and complexes detected; e.g., using an immobilized antibody specific for a GPC receptor or the test agent to anchor any complexes formed in solution, and a labeled antibody specific for the other component of the possible complex to detect anchored complexes.

In one embodiment, the screening method comprises: a) incubating an HGPRBMY3 polypeptide with an HGPRBMY3-binding peptide under conditions to form a complex; b) incubating the complex of (a) with a test agent; and c) screening for disruption of the complex, wherein disruption of the complex indicates identification of HGPRBMY3-binding agent. In another embodiment, the method comprises: a) incubating an HGPRBMY3 polypeptide with a test agent; b) adding an HGPRBMY3-binding peptide to the polypeptide and test agent of (a); and c) screening for formation of a complex between the polypeptide and the peptide, wherein inhibition of formation of the complex indicates identification of an HGPRBMY3-binding agent.

In a further embodiment, the screening method comprises: a) incubating an HGPRBMY3 polypeptide with an HGPRBMY3-binding anti-idiotype antibody under conditions to form a complex; b) incubating the complex of (a) with a test agent; and c) screening for disruption of the complex, wherein disruption of the complex indicates identification of HGPRBMY3-binding agent. In yet another embodiment, the method comprises: a) incubating an HGPRBMY3 polypeptide with a test agent; b) adding an HGPRBMY3-binding anti-idiotype antibody to the polypeptide and test agent of (a); and c) screening for formation of a complex between the polypeptide and the antibody, wherein inhibition of formation of the complex indicates identification of an HGPRBMY3-binding agent.

In vitro systems can be designed to identify agents capable of binding to HGPRBMY3 or a related GPC receptor (e.g., SEQ ID NO:2, SEQ ID NO:110-SEQ ID NO:112, or a fragment thereof). In one embodiment, the in vitro assays involve a high-throughput format, where an HGPRBMY3 polypeptide or peptide, cell or tissue expressing this polypeptide or peptide is attached to a solid phase substrate. In such high-throughput assays, it is possible to screen up to several thousand different modulators or ligands in a single day. In practice, microtiter plates can conveniently be utilized for solid phase screening. The anchored components can be immobilized by non-covalent or covalent attachments. Non-covalent attachment can be accomplished by simply coating the solid surface with a solution of the protein or peptide and drying. Alternatively, an immobilized antibody, preferably a monoclonal antibody (e.g., an anti-idiotype antibody), specific for the HGPRBMY3 polypeptide or peptide can be used to anchor the polypeptide or peptide to the solid surface. The surfaces can be prepared in advance and stored.

In accordance with the invention, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536-well plates are used, then a single plate can easily assay from about 100 to about 1500 different compounds. It is possible to assay several different plates per day; thus, for example, assay screens for up to about 6,000-20,000 different compounds are possible using the described integrated systems.

In one aspect, the binding assays can include preparation of a reaction mixture of HGPRBMY3 (e.g., SEQ ID NO:2), or a related GPC receptor (e.g., SEQ ID NO:110-SEQ ID NO:112), or peptide and the test agent under conditions and for a time sufficient to allow the two components to interact and bind, thus forming a complex. A disclosed antibody, anti-idiotype antibody, peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof), or polypeptide can then be added as a binding competitor, and any remaining HGPRBMY3-test agent complex can be removed and/or detected in the reaction mixture. These assays can be carried out in a variety of ways. As examples, the HGPRBMY3 polypeptide or the test substance can be anchored onto a solid phase, and HGPRBMY3-test agent complexes anchored onto the solid phase can be detected at the end of the reaction. In one specific aspect, an HGPRBMY3 polypeptide can be anchored onto a solid surface, and the test agent, which is not anchored, can be labeled, either directly or indirectly.

In order to conduct the binding assays of the invention, the non-immobilized components are added to the coated surface containing the anchored components. After the reaction is complete, any unbound components are removed, e.g., by washing. Washing conditions are designed allow any complexes to remain immobilized on the solid surface. The detection of complexes anchored on the solid surface can be accomplished in a number of ways. Where the previously immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

One useful technique provides for high-throughput screening of agents having suitable binding affinity to the protein of interest (see, e.g., WO 84/03564 to Venton, et al.). In this method, large numbers of different small test agents are synthesized on a solid substrate, such as plastic pins or some other surface. The test agents are a) reacted with a GPC receptor or fragments thereof; b) competed with a disclosed antibody, anti-idiotype antibody, peptide, or polypeptide; and c) washed. Bound GPC receptor is then detected by methods well known in the art. Purified GPC receptor can also be coated directly onto plates for use in the aforementioned drug screening techniques. Alternatively, non-neutralizing antibodies can be used to capture a GPC receptor and immobilize it on a solid support. In a further embodiment of this invention, competitive drug screening assays can be used in which neutralizing anti-idiotype antibodies, capable of binding to a GPC receptor, specifically compete with a test agent for binding to the receptor. Using this technique, the antibodies can be used to detect the presence of any agent that shares one or more antigenic determinants with the GPC receptor.

Another useful technique provides cell membrane-based scintillation proximity assays (“SPAs”) to identify GPCR binding agents (e.g., ligands). SPA-based assays are well known in the art (see, e.g., U.S. Pat. No. 4,568,649, which is incorporated herein by reference), and are commercially available. For example, kits for [³H] SPA 5-HT₇ Receptor Binding Assays and SPA G-Protein Coupled Receptor Assays are available from Amersham. In some cases, certain modifications of known SPA assays may be required to adapt such assays to use with a GPC receptor, or peptide fragment thereof. Such modifications are also encompassed by the present invention. Generally, the SPAs of the invention use microscopic beads that are coupled to cell membranes and contain scintillant that can be stimulated to emit light. This stimulation event only occurs when radiolabeled binding agents are bound to the surface of the beads. For the assay, SPA beads and membranes are added first, and then radiolabeled binding agents are added. A peptide (e.g., any one of SEQ ID NO:17-SEQ ID NO:26, SEQ ID NO:35-SEQ ID NO:46, and SEQ ID NO:73-SEQ ID NO:77, or a fragment, variant, or derivative thereof), polypeptide, or anti-idiotype antibody is then added as a binding competitor. After an equilibration period of 2-4 h at room temperature, the plates are counted in a scintillation counting machine, and the percent stimulation is calculated. Once identified, unlabeled ligand can be added to assay-ready plates to serve as a positive control.

SPA assays may be based upon a manual, automated, or semi-automated platforms, and encompass 96-, 384-, 1536-well plates or larger formats. Any number of SPA beads may be used as applicable to each assay. Examples of SPA beads include, for example, Leadseeker WGA PS (Amersham cat # RPNQ 0260), and SPA Beads (PVT-PEI-WGA-TypeA; Amersham cat # RPNQ00O3). The membranes for SPAs may be derived from a number of cell line and tissue sources depending upon the expression profile of the respective polypeptide and the adaptability of such a cell line or tissue source to the development of a SPA-based assay. Examples of membrane preparations include, for example, cell lines transformed to express the receptor to be assayed in CHO cells or HEK cells.

Another example of a useful binding assay is the fluorescence-based thermal shift assay (3-Dimensional Pharmaceuticals, Inc., 3DP, Exton, Pa.; as described in U.S. Pat. Nos. 6,020,141 and 6,036,920 to Pantoliano et al.; see also, J. Zimmerman, 2000, Gen. Eng. News, 20(8)). This assay allows the detection of test agents (e.g., drugs, ligands) that bind to the expressed, and preferably purified, GPC receptor, or fragment thereof. Binding is determined by analyzing thermal unfolding curves of protein-drug or protein-ligand complexes.

As described herein, cell-based panning of phage-display peptide libraries can be used to identify agents (e.g., peptides) that bind to a GPC receptor. Typically, panning experiments are optimized so that target-specific phage are amplified more efficiently than phage that bind irrelevant endogenous cell surface epitopes. Common strategies for optimization involve the use of a) competitive ligands; b) alternating cell lines between panning cycles (e.g., alternating between Sf9 and CHO or COS); and c) preadsorbing phage libraries against target-negative parental cell line. However, in some cases, strategies (a) may not be possible (e.g., due lack of competitive ligands for orphan receptors), and strategy (b) may not be efficient (e.g., requiring expression of the target in multiple cell lines).

Therefore, an improved cell-based panning protocol has been disclosed herein based on an effective preadsorption approach. The protocol is highly effective, based on the identification of HGPRBMY3-binding peptides from only ˜100 phage in the final eluate. Target-specificity is indicated by the fact that peptides comprising the disclosed triplet motifs (KIW, KVW, KLW, or RVW; see FIG. 18A) were not recovered from panning experiments using the parental cell line, or cells expressing HGPRBMY2 (U.S. Ser. No. 10/081,810, filed Feb. 2, 2002 which is hereby incorporated by reference in its entirety), HGPRBMY6 (U.S. Ser. No. 09/966,422, filed Sep. 26, 2001, which is hereby incorporated by reference in its entirety), HGPRBMY9 (U.S. Ser. No. 09/964,923, filed Sep. 26, 2001, which is hereby incorporated by reference in its entirety), HGPRBMY14 (U.S. Ser. No. 10/067,649, filed Feb. 5, 2002, which is hereby incorporated by reference in its entirety), HGPRBMY17 (Genbank Accession No. NP_{—}004942).

Importantly, the panning protocol does not make use of any specialized reagents. In principle, therefore, it can be utilized with any targeted cell surface protein. In specific aspects, the panning protocol is preferably used with certain cell lines, such as CHO-K1. Other cell lines, such as HEK293, are not preferred. In addition, multiple (e.g., two) preadsorptions against the parental cell line are preferred. For optimal results, preadsorption against purified integrin can be performed. The integrin preadsorption step is useful for screens using linear peptide libraries, and essential for screens using with cyclic peptide libraries. The cell-based panning protocol is preferably performed using phage-based libraries such as fUSE5-based 15-mer and M13-based 12- and 15-mer libraries, but other linear and cyclic libraries, and monovalent phagemid libraries, can also be used.

For the binding-based identification, panning, or screening assays of the invention, a functional assay is not typically required. Only a target protein is needed, preferably substantially purified, and a library or panel of compounds (e.g., ligands, drugs, small molecules) or biological entities to be screened or assayed for binding to the target. Preferably, most small molecules that bind to the target protein will modulate activity in some manner, due to preferential, higher affinity binding to functional areas or sites on the protein. Thus, the drugs, ligands, or binding agents determined by any of these assays can be further tested, if desired, by methods, such as those described herein, to determine if the molecules affect or modulate function or activity of the target protein. Agents identified by the disclosed methods can be useful, for example, in: a) modulating the activity of wild type and/or mutant GPC receptor; b) elaborating the biological function of the GPC receptor; c) screens for identifying additional agents that disrupt normal GPC receptor interactions, or can in themselves disrupt such interactions.

### Competitive Assays

In accordance with one method, binding agents can be identified by assessing competition with labeled binders (e.g., antibodies, anti-idiotype antibodies, peptides, such as SEQ ID NO:17-SEQ ID NO:26, SEQ ID NO:35-SEQ ID NO:46, SEQ ID NO:73-SEQ ID NO:77, polypeptides, and fragments, variants, and derivatives thereof) using cells which express the receptor on the cell surface, or cell membranes comprising the receptor. For this method, a eukaryotic cell is transfected with DNA encoding an HGPRBMY3 polypeptide (e.g., SEQ ID NO:2) or another GPC receptor (e.g., SEQ ID NO:110-SEQ ID NO:112). The cell expresses the GPC receptor on its surface. The cell is then contacted with a test agent in the presence of a labeled binder, such as an HGPRBMY3-binding peptide, polypeptide, or anti-idiotype antibody. In various embodiments, the binder is labeled, e.g., by radioactivity, fluorescence, or any detectable label commonly known in the art. The amount of labeled binder bound to the receptors is measured, for example, by measuring radioactivity associated with transfected cells or membrane from these cells. If the test agent binds to the receptor, the binding of labeled ligand to the receptor may be decreased.

In another method, mammalian cells, for example, but not limited to, CHO, HEK 293, Xenopus oocytes, RBL-2H3, etc., are transfected and used to express HGPRBMY3. The cells are loaded with an indicator dye that produces a fluorescent signal when bound to calcium, and the cells are contacted with a test substance and a receptor ligand, such as dATP, dAMP, or UTP, or a GPCR-binding peptide, polypeptide, or anti-idiotype antibody. Any change in fluorescent signal is measured over a defined period of time using, for example, a fluorescence spectrophotometer or a fluorescence imaging plate reader. A change in the fluorescence signal pattern generated by the ligand indicates that the agent is a potential antagonist or agonist for the receptor.

In yet another screening method, mammalian cells are transfected to express HGPRBMY3 (e.g., SEQ ID NO:2) or a related GPC receptor (e.g., SEQ ID NO:110-SEQ ID NO:112), and are also transfected with a reporter gene construct that is coupled to activation of the receptor. As non-limiting examples, luciferase and β-galactosidase reporter genes can be used with appropriate promoters. The cells are contacted with a test agent and a receptor ligand, such as dATP, dAMP, or UTP, or a GPCR-binding peptide, polypeptide, or anti-idiotype antibody. The signal produced by the reporter gene is measured after a defined period of time. The signal can be measured using a luminometer, spectrophotometer, fluorimeter, or other such instrument appropriate for the specific reporter construct used. An increase or decrease of the signal generated by the ligand indicates that a agent is a potential agonist or antagonist for the receptor.

Another screening method involves introducing RNA encoding HGPRBMY3 or anther GPC receptor into cells (e.g., CHO, HEK 293, RBL-2H3, etc.) to transiently or stably express the receptor. The cells are then contacted with a receptor ligand, such as dATP, dAMP, or UTP, or a GPCR-binding peptide, polypeptide, or anti-idiotype antibody, and a test agent to be screened. Inhibition or activation of the receptor is then determined by detection of a signal, such as, cAMP, calcium, protons, or other ions.

One useful screening method involves the use of melanophores which are transfected to express HGPRBMY3 or a related GPC receptor (see, e.g., PCT WO 92/01810, published Feb. 6, 1992). The melanophore cells that express the GPC receptor are contacted with both the receptor ligand, such as LPA, or a GPCR-binding peptide, polypeptide, or anti-idiotype antibody, and an agent to be screened. Inhibition of the signal generated by the ligand indicates that an agent is a potential antagonist for the receptor, i.e., inhibits activation of the receptor.

### Functional Assays

Further encompassed are methods of using the disclosed antibodies, anti-idiotype antibodies, peptides (e.g., SEQ ID NO:17-SEQ ID NO:26, SEQ ID NO:35-SEQ ID NO:46, and SEQ ID NO:73-SEQ ID NO:77, and fragments, variants, and derivatives thereof), and polypeptides to screen for other agents that modulate HGPRBMY3 (e.g., SEQ ID NO:2) or a related GPC receptor (e.g., SEQ ID NO:110-SEQ ID NO:112). For example, agents that modulate the receptor can be identified by contacting cells expressing the GPC receptor with agents to be screened and determining whether such agent increases or decreases the signal, i.e., activates or inhibits the receptor. Other modulating agents can be identified using cells which express the GPC receptor (for example, transfected CHO cells) in a system which measures extracellular pH changes caused by receptor activation. To determine inhibition or activation of a GPC receptor, a second messenger response can be measured, e.g., signal transduction or pH changes.

One method for identifying modulators comprises combining a binding agent with HGPRBMY3 (e.g., SEQ ID NO:2), a related GPC receptor (e.g., SEQ ID NO:110-SEQ ID NO:112), or a fragment thereof, and measuring an effect of the agent on the biological activity of the GPC receptor or receptor fragment. Such measurable effects include, for example, physical binding interaction; the ability to cleave a suitable GPC receptor substrate; effects on native and cloned GPCR-expressing cell line; and effects of modulators or other GPC receptor-mediated physiological measures.

Another method for identifying agents that modulate the biological activity of the novel HGPRBMY3 polypeptides of the present invention comprises combining a binding agent with a host cell that expresses HGPRBMY3 (e.g., SEQ ID NO:2), a related GPC receptor, or a fragment thereof, and measuring an effect of the agent on the biological activity of the GPC receptor. The host cell can also be capable of being induced to express the GPC receptor, e.g., via inducible expression. The physiological effects of a modulator on GPC receptor activity can also be measured. Cellular assays for particular GPC receptor modulators may be either direct (e.g., measurement or quantification of the biological activity of the GPC receptor) or indirect (e.g., measurement or quantification of a physiological effect). Such methods preferably employ HGPRBMY3 or a related GPC receptor as described herein, or a recombinant GPC receptor in suitable host cells containing an expression vector as described herein, wherein the GPC receptor is expressed, overexpressed, or undergoes regulated (i.e., inducible) expression.

One specific method for identifying modulatory agents comprises: a) providing a host cell containing an expression vector harboring a nucleic acid sequence encoding HGPRBMY3 polypeptide (e.g., SEQ ID NO:2), or a functional peptide or portion thereof; b) determining the biological activity of the expressed HGPRBMY3 polypeptide in the absence of a test agent; c) contacting the cell with a test agent, and determining the biological activity of the expressed HGPRBMY3 polypeptide in the presence of the modulator agent. In such a method, a difference between the activity of the HGPRBMY3 polypeptide in the presence of the modulator agent and in the absence of the modulator agent indicates a modulating effect of the agent.

In one embodiment of the invention, the screening method comprises: a) identifying an agent that binds to an HGPRBMY3 polypeptide (see above); and b) testing the agent for agonist or antagonist activity, wherein observation of this activity indicates identification of an agonist or antagonist agent. In another embodiment, the screening method comprises: a) incubating an HGPRBMY3 polypeptide with an agonist peptide; b) incubating the polypeptide and peptide of (a) with a test agent; and c) testing for a reversal of the agonist effect of the peptide, wherein observation of this reversal indicates identification of an antagonist agent. In a further embodiment, the screening method comprises: a) incubating an HGPRBMY3 polypeptide with an antagonist peptide; b) incubating the polypeptide and peptide of (a) with a test agent; and c) testing for a reversal of the antagonist effect of the peptide, wherein observation of this reversal indicates identification of an agonist agent. The anti-idiotype antibodies of the invention can be used for similar screens.

Preferably, mammalian cell reporter assays can be used demonstrate functional coupling of HGPRBMY3 or a related GPC receptor in accordance with well-established techniques (Gilman, 1987, Boss et al., 1996; Alam & Cook, 1990; George et al., 1997; Selbie & Hill, 1998; Rees et al., 1999). Such assays have been successfully used to identify novel small molecule agonists or antagonists for GPC receptors, and a class of drug targets (Zlokarnik et al., 1998; George et al., 1997; Boss et al., 1996; Rees et al., 2001). In these assays, a promoter is regulated by activation of specific signal transduction cascades following agonist binding to a GPC receptor (Alam & Cook 1990; Selbie & Hill, 1998; Boss et al., 1996; George et al., 1997; Gilman, 1987). A number of response element-based reporter systems have been developed to study of GPC receptor function. These include cAMP response element (CRE)-based reporter genes for Gαi/o or Gas-coupled GPC receptors, nuclear factor activator of transcription (NFAT)-based reporters for Gαq/11-coupled receptors, and MAP kinase reporter genes for Gαi/o coupled receptors (Selbie & Hill, 1998; Boss et al., 1996; George et al., 1997; Gilman, 1987; Rees et al., 2001).

As demonstrated herein, transcriptional response elements that regulate the expression of beta-lactamase in CHO K1 cells (CHO-NFAT/CRE: Aurora Biosciences) (Zlokarnik et al., 1998) have been used to characterize the function of the HGPRBMY3 polypeptide of the invention. This system allows demonstration of constitutive G-protein coupling to endogenous cellular signaling components upon intracellular overexpression of orphan receptors. Overexpression of GPC receptors has been shown to be physiologically important. For the CCR2 receptor, it has been shown that overexpression occurs in metastatic carcinomas, and defective expression in macrophages is associated with increased incidence of human ovarian carcinoma (Sica, et al., 2000; Salcedo et al., 2000). In addition, overproduction of the beta-2 adrenergic receptor in transgenic mice leads to constitutive activation of the receptor signaling pathway, and increased cardiac output (Kypson et al., 1999; Dorn et al., 1999). These are only a few of the many examples demonstrating constitutive activation of GPC receptors that are likely to be in the active, R*, conformation (J. Wess, 1997).

In preferred embodiments, CHO-NFAT/CRE cells transfected with HGPRBMY3 or a related GPC receptor are useful for the identification of agonists and antagonists of the GPC receptor. Preferably, the cell lines are used in methods comprising: a) combining a candidate modulator agent with a host cell expressing the HGPRBMY3 (e.g., SEQ ID NO:2), or fragment thereof; and b) measuring an effect of the candidate modulator agent on the activity of the expressed HGPRBMY3 polypeptide. Alternatively, the cell lines are used in methods comprising: a) determining the biological activity of the HGPRBMY3 polypeptide or peptide in the absence of a modulator agent; b) contacting a host cell expressing the HGPRBMY3 polypeptide or peptide with the modulator agent; and c) determining the biological activity of the HGPRBMY3 polypeptide or peptide in the presence of the modulator agent; wherein a difference between the activity of the HGPRBMY3 polypeptide or peptide in the presence of the modulator agent and in the absence of the modulator agent indicates a modulating effect of the agent. Representative vectors expressing the HGPRBMY3 polypeptide are referenced herein (e.g., pcDNA3.1 Hygro™), and are otherwise known in the art. Additional uses for these cell lines are described herein and known in the art, and are also encompassed by the present invention.

### Signal Transduction Assays

In various aspects of the invention, the signal transduction activity of HGPRBMY3 or a related GPC receptor may be monitored by measuring levels of intracellular Ca²⁺, cAMP, inositol 1,4,5-triphosphate (IP₃), and/or 1,2-diacylglycerol (DAG). Activation of a GPC receptor triggers the release of Ca²⁺ ions from the mitochondria, endoplasmic reticulum, and other cytoplasmic vesicles into the cytoplasm. Fluorescent dyes, e.g., fura-2, can be used to measure the concentration of free cytoplasmic Ca²⁺. For example, the ester of fura-2, which is lipophilic and can diffuse across the cell membrane, can be added to the media of the host cells expressing a GPC receptor. Once inside the cell, the fura-2 ester is hydrolyzed by cytosolic esterases to its non-lipophilic form, and then the dye cannot diffuse back out of the cell. The non-lipophilic form of fura-2 fluoresces when it binds to free Ca²⁺. The fluorescence can be measured without lysing the cells at an excitation spectrum of 340 nm or 380 nm and at fluorescence spectrum of 500 nm (Sakurai et al., EP 480 381).

Upon activation of a GPC receptor, the increase in free cytosolic Ca²⁺ concentrations is preceded by the hydrolysis of phosphatidylinositol 4,5-bisphosphate. Hydrolysis of this phospholipid by phospholipase C yields 1,2-diacylglycerol (DAG), which remains in the membrane, and water-soluble inositol 1,4,5-triphosphate (IP₃). Binding of ligands or agonists will increase the concentration of DAG and IP₃. To measure the IP₃ concentrations, radioactivity labeled ³H-inositol can be added to the media of host cells expressing GPC receptors. The ³H-inositol is taken up by the cells and incorporated into IP₃. The resulting inositol triphosphate can be separated from the mono and di-phosphate forms and measured (Sakurai et al., EP 480 381). Alternatively, Amersham (Arlington Heights, Ill.) provides an inositol 1,4,5-triphosphate assay system. With this system, Amersham provides tritylated inositol 1,4,5-triphosphate and a receptor capable of distinguishing the radioactive inositol from other inositol phosphates. These reagents can be used in an effective and accurate competition assay to determine the IP₃ levels. Cyclic AMP levels can be measured according to any of the methods known in the art, for example, the methods described in Gilman et al., Proc. Natl. Acad. Sci 67:305-312 (1970). In addition, a kit for assaying levels of cAMP is available from Diagnostic Products Corp. (Los Angeles, Calif.).

One method for identifying modulators involves using eukaryotic cells to express HGPRBMY3 (e.g., SEQ ID NO:2) or a related GPC receptor (e.g., SEQ ID NO:110-SEQ ID NO:112) fused to phospholipase C or D. Representative examples of such cells include, but are not limited to, endothelial cells, smooth muscle cells, and embryonic kidney cells. Screening may be accomplished by detecting activation of the receptor or inhibition of activation of the receptor from the phospholipase second signal, as described in detail herein.

Another method involves screening for modulators by determining the increase or decrease of GPC receptor-mediated cAMP and/or adenylate cyclase production. This method involves transiently or stably transfecting eukaryotic cells with HGPRBMY3 or a related GPC receptor to express the receptor on the cell surface. The cell is then exposed to a test agent in the presence of a receptor ligand, such as a GPCR-binding peptide, polypeptide, or anti-idiotype antibody. The changes in levels of cAMP is then measured over a defined period of time, for example, by radioimmuno or protein binding assays (for example using Flashplates or a scintillation proximity assay). Changes in cAMP levels can also be determined by directly measuring the activity of the enzyme, adenylyl cyclase, in broken cell preparations. If the potential antagonist or agonist binds the receptor, and thus inhibits binding of the GPCR ligand, the levels of GPC receptor-mediated cAMP, or adenylate cyclase activity, will be increased or decreased.

One preferred screening method involves co-transfecting HEK-293 cells with a mammalian expression plasmid encoding a GPC receptor, such as HGPRBMY3, along with a mixture comprised of mammalian expression plasmids cDNAs encoding GUI5 (Wilkie T. M. et al. Proc Natl Acad Sci USA 1991 88: 10049-10053), GU16 (Amatruda T. T. et al. Proc Natl Acad Sci USA 1991 8: 5587-5591), and three chimeric G-proteins referred to as Gqi5, Gqs5, and Gqo5 (Conklin BR et al., Nature 1993 363: 274-276, Conklin B. R. et al Mol Pharmacol 1996 50:885-890). Following a 24 h incubation, the transfected HEK-293 cells are plated into poly-D-lysine coated 96-well black/clear plates (Becton Dickinson, Bedford, Mass.).

Following addition of test agents, the cells are assayed on FLIPR (Fluorescent Imaging Plate Reader, Molecular Devices, Sunnyvale, Calif.) for a calcium mobilization response. Upon identification of a ligand that stimulates calcium mobilization in HEK-293 cells expressing the GPC receptor and the G-protein mixtures, subsequent experiments are performed to determine which, if any, G-protein is required for the functional response. HEK-293 cells are then transfected with a GPC receptor, or co-transfected with the receptor and G015, GD16, GqiS, GqsS, or Gqo5. If the GPC receptor requires the presence of one of the G-proteins for functional expression in HEK-293 cells, all subsequent experiments are performed with HEK-293 cell cotransfected with the GPC receptor and the G-protein that gives the best response. Alternatively, the receptor can be expressed in a different cell line, for example RBL-2H3, without additional G-proteins.

### Pheromone Response Assays

Another screening method for modulators relies on the endogenous pheromone response pathway in the yeast, Saccharomyces cerevisiae. Heterothallic strains of yeast can exist in two mitotically stable haploid mating types, MATα and MATa. Each cell type secretes a small peptide hormone that binds to a yeast GPC receptor on opposite mating type cells. This binding triggers a MAP kinase cascade leading to G1 arrest as a prelude to cell fusion. Heterologous expression and coupling of human GPC receptors and humanized G-protein subunits in yeast cells can be linked to downstream signaling pathways and reporter genes (see, e.g., U.S. Pat. Nos. 5,063,154; 5,482,835; 5,691,188).

Genetic alteration of certain genes in the pheromone response pathway can alter the normal response to pheromone. Such genetic alterations include, but are not limited to, (i) deletion of the STE2 or STE3 gene encoding the endogenous GPC pheromone receptors; (ii) deletion of the FAR1 gene encoding a protein that normally associates with cyclin-dependent kinases leading to cell cycle arrest; and (iii) construction of reporter genes fused to the FUS 1 gene promoter, where FUS 1 encodes a membrane-anchored glycoprotein required for cell fusion. Downstream reporter genes can permit either a positive growth selection (e.g., histidine prototrophy using the FUS1-HIS3 reporter), or a colorimetric, fluorometric or spectrophotometric readout, depending on the specific reporter construct used (e.g., β-galactosidase induction using a FUS 1-LacZ reporter).

The yeast cells can be further engineered to express and secrete small peptides from random peptide libraries, some of which can permit autocrine activation of heterologously expressed human (or mammalian) GPC receptors (Broach, J. R. and Thorner, J., Nature 384: 14-16, 1996; Manfredi et al., Mol. Cell. Biol. 16: 4700-4709,1996). This provides a rapid direct growth selection (e.g., using the FUS1-HIS3 reporter) for surrogate peptide agonists that activate orphan or characterized GPC receptors. Alternatively, yeast cells that functionally express human (or mammalian) GPC receptors linked to a reporter gene readout (e.g., FUS1-LacZ) can be used as a platform for high-throughput screening of known ligands, fractions of biological extracts, and libraries of chemical compounds for either natural or surrogate ligands. Functional agonists of sufficient potency (whether natural or surrogate) can be used as screening tools in yeast cell-based assays for identifying GPC receptor antagonists. For example, agonists, such as GPCR-binding peptides, polypeptides, or anti-idiotype antibodies, will promote growth of a cell with FUS-HIS3 reporter or give positive readout for a cell with FUS1-LacZ. In contrast, an agent that inhibits growth or negates the positive readout induced by the agonist is an antagonist. For this purpose, the yeast system offers advantages over mammalian expression systems due to its ease of utility and its lack of endogenous GPC receptors.

### EMBODIMENTS OF THE INVENTION

The invention encompasses, but is not limited to, the following embodiments:

An isolated nucleic acid encoding an amino acid sequence selected from the group consisting of SEQ ID NO:17-SEQ ID NO:26, SEQ ID NO:35-SEQ ID NO:46, and SEQ ID NO:73-SEQ ID NO:77.

An isolated nucleic acid comprising a nucleotide sequence selected from the group consisting of SEQ ID NO:47-SEQ ID NO:63, SEQ ID NO:67-SEQ ID NO:71, SEQ ID NO:78-SEQ ID NO:99, SEQ ID NO:100-SEQ ID NO:104, and SEQ ID NO:105-SEQ ID NO:109.

An isolated nucleic acid comprising at least 20 contiguous nucleotides of a nucleotide sequence selected from the group consisting of SEQ ID NO:47-SEQ ID NO:63, SEQ ID NO:67-SEQ ID NO:71, SEQ ID NO:78-SEQ ID NO:99, SEQ ID NO:100-SEQ ID NO:104, and SEQ ID NO:105-SEQ ID NO:109.

An isolated nucleic acid comprising a nucleotide sequence that is at least 56% identical to the nucleotide sequence of any one of the preceding embodiments.

An isolated nucleic acid comprising a nucleotide sequence that is complementary to the nucleotide sequence of any one of the preceding embodiments.

A vector comprising the isolated nucleic acid of any one of the preceding embodiments.

A host cell comprising the vector of any one of the preceding embodiments, wherein the host cell is selected from the group consisting of bacterial, yeast, insect, mammalian, and plant cells.

A primer comprising the isolated nucleic acid of any one of the preceding embodiments.

A probe comprising the isolated nucleic acid of any one of the preceding embodiments.

An isolated peptide comprising an amino acid sequence selected from the group consisting of SEQ ID NO:17-SEQ ID NO:26, SEQ ID NO:35-SEQ ID NO:46, and SEQ ID NO:73-SEQ ID NO:77.

An isolated peptide comprising at least 8 contiguous amino acids of an amino acid sequence selected from the group consisting of SEQ ID NO:17-SEQ ID NO:26, SEQ ID NO:35-SEQ ID NO:46, and SEQ ID NO:73-SEQ ID NO:77.

An isolated peptide comprising an amino acid sequence that is at least 84% identical to the amino acid sequence of any one of the preceding embodiments.

An antibody that binds to the isolated peptide of any one of the preceding embodiments. In specific embodiments, the antibody is monoclonal.

An anti-idiotype antibody raised to the monoclonal antibody of any one of the preceding embodiments, which binds to a P2Y-type G-protein coupled receptor.

An anti-idiotype antibody raised to the monoclonal antibody of any one of the preceding embodiments, which binds to a G-protein coupled receptor selected from the group consisting of P2Y10, HGPRBMY3, HGPRBMY11, and HGPRBMY23. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively.

A polypeptide complex comprising a P2Y-type G-protein coupled receptor, and a peptide comprising an amino acid sequence selected from the group consisting of SEQ ID NO:17-SEQ ID NO:26 AND SEQ ID NO:35-SEQ ID NO:46, and SEQ ID NO:73-SEQ ID NO:77.

A polypeptide complex comprising a G-protein coupled receptor selected from the group consisting of P2Y10, HGPRBMY3, HGPRBMY11, and HGPRBMY23, and a peptide comprising an amino acid sequence selected from the group consisting of SEQ ID NO:17-SEQ ID NO:26, SEQ ID NO:35-SEQ ID NO:46, and SEQ ID NO:73-SEQ ID NO:77. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively

An antibody that binds to the polypeptide complex of any one of the preceding embodiments. In specific embodiments, the antibody is monoclonal.

A method of isolating a peptide comprising an amino acid sequence selected from the group consisting of SEQ ID NO:17-SEQ ID NO:26 SEQ ID NO:35-SEQ ID NO:46, and SEQ ID NO:73-SEQ ID NO:77 comprising: incubating the antibody of any one of the preceding embodiments with the peptide, thereby isolating the peptide.

A peptide library generated from the isolated nucleic acid of any one of the preceding embodiments. In specific embodiments, the peptide library is a phage display peptide library.

A method of identifying a binding agent for a P2Y-type G-protein coupled receptor comprising: screening the peptide library of any one of the preceding embodiments for one or more peptides that bind to a G-protein coupled receptor, wherein binding indicates identification of an G-protein coupled receptor-binding agent.

A method of identifying a binding agent for a G-protein coupled receptor selected from the group consisting of P2Y10, HGPRBMY3, HGPRBMY11, and HGPRBMY23, comprising: screening the peptide library of any one of the preceding embodiments for one or more peptides that bind to P2Y10, HGPRBMY3, HGPRBMY11, or HGPRBMY23, wherein binding indicates identification of a binding agent. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively

A method of identifying a P2Y-type G-protein coupled receptor comprising:

1) incubating the isolated peptide or antibody of any one of the preceding embodiments with a biological sample under conditions that allow the peptide or antibody to bind to a G-protein coupled receptor in the sample, and thereby form a complex; and

2) identifying formation of the complex in (1), wherein formation of the complex indicates identification of a G-protein coupled receptor.

A method of identifying a G-protein coupled receptor selected from the group consisting of P2Y10, HGPRBMY3, HGPRBMY11, and HGPRBMY23, comprising:

1) incubating the isolated peptide or antibody of any one of the preceding embodiments with a biological sample under conditions that allow the peptide or antibody to bind to P2Y10, HGPRBMY3, HGPRBMY11, or HGPRBMY23 in the sample, and thereby form a complex; and

2) identifying formation of the complex in (1), wherein formation of the complex indicates identification of P2Y10, HGPRBMY3, HGPRBMY11, or HGPRBMY23. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively

A method of identifying binding agent for a P2Y-type G-protein coupled receptor comprising:

1) incubating the isolated polypeptide complex of any one of the preceding embodiments with a test agent under conditions that allow the test agent to bind to the complex; and

2) screening for disruption of the polypeptide complex, wherein disruption of the complex indicates identification of a binding agent.

A method of identifying a binding agent for a G-protein coupled receptor selected from the group consisting of P2Y10, HGPRBMY3, HGPRBMY11, and HGPRBMY23 comprising:

1) incubating the isolated polypeptide complex of any one of the preceding embodiments with a test agent under conditions that allow the test agent to bind to the complex; and

2) screening for disruption of the polypeptide complex, wherein disruption of the complex indicates identification of a binding agent. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively

A method of identifying binding agent for a P2Y-type G-protein coupled receptor comprising:

1) incubating a G-protein coupled receptor with a test agent;

2) incubating the receptor and test agent with the isolated peptide of any one of the preceding embodiments; and

3) screening for formation of a polypeptide complex between the receptor and the isolated peptide, wherein inhibition of formation of the complex indicates' identification of a binding agent.

A method of identifying binding agent for a G-protein coupled receptor selected from the group consisting of P2Y10, HGPRBMY3, HGPRBMY11, and HGPRBMY23, comprising:

1) incubating a G-protein coupled receptor, P2Y10, HGPRBMY3, HGPRBMY11, or HGPRBMY23, with a test agent;

2) incubating the receptor and test agent with the isolated peptide of any one of the preceding embodiments; and

3) screening for formation of a polypeptide complex between the receptor and the isolated peptide, wherein inhibition of formation of the complex indicates identification of a binding agent. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively A method of identifying a antagonist agent for a P2Y-type G-protein coupled receptor comprising:

1) incubating a G-protein coupled receptor with the isolated peptide or antibody from any one of the preceding embodiments which has agonist activity;

2) incubating the G-protein coupled receptor and the peptide or antibody of (a) with a test agent; and

3) testing for a reversal of the agonist effect of the peptide or antibody, wherein observation of this reversal indicates identification of an antagonist agent.

A method of identifying a antagonist agent for a G-protein coupled receptor selected from the group consisting of P2Y10, HGPRBMY3, HGPRBMY11, and HGPRBMY23, comprising:

1) incubating a G-protein coupled receptor, P2Y10, HGPRBMY3, HGPRBMY11, or HGPRBMY23, with the isolated peptide or antibody from any one of the preceding embodiments which has agonist activity;

2) incubating the G-protein coupled receptor and the peptide or antibody of (a) with a test agent; and

3) testing for a reversal of the agonist effect of the peptide or antibody, wherein observation of this reversal indicates identification of an antagonist agent. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively.

A method of identifying a agonist agent for a P2Y-type G-protein coupled receptor comprising:

1) incubating a G-protein coupled receptor with the isolated peptide or antibody from any one of the preceding embodiments which has antagonist activity;

2) incubating the G-protein coupled receptor and the peptide or antibody of (a) with a test agent; and

3) testing for a reversal of the antagonist effect of the peptide or antibody, wherein observation of this reversal indicates identification of an agonist agent.

A method of identifying a agonist agent for a G-protein coupled receptor selected from the group consisting of P2Y10, HGPRBMY3, HGPRBMY11, and HGPRBMY23, comprising:

1) incubating a G-protein coupled receptor, P2Y10, HGPRBMY3, HGPRBMY11, or HGPRBMY23, with the isolated peptide or antibody from any one of the preceding embodiments which has antagonist activity;

2) incubating the G-protein coupled receptor and the peptide or antibody of (a) with a test agent; and

3) testing for a reversal of the antagonist effect of the peptide or antibody, wherein observation of this reversal indicates identification of an agonist agent. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively.

A kit for detecting a P2Y-type G-protein coupled receptor comprising:

1) the isolated peptide or antibody of any one of the preceding embodiments; and

2) one or more reagents for detecting binding of the receptor and the peptide or antibody.

A kit for detecting a G-protein coupled receptor selected from the group consisting of P2Y10, HGPRBMY3, HGPRBMY11, and HGPRBMY23, comprising:

1) the isolated peptide or antibody of any one of the preceding embodiments; and

2) one or more reagents for detecting binding of the receptor and the peptide or antibody. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively.

A method of diagnosing a proliferative disorder comprising:

1) incubating the peptide or antibody of any one of the preceding embodiments with a biological sample under conditions to allow the peptide or antibody to associate with a P2Y-type G-protein coupled receptor in the sample; and

2) measuring levels of peptide-receptor or antibody-receptor complex formed in (1), wherein an alteration in these levels compared to standard levels indicates diagnosis of the proliferative disorder.

A method of diagnosing a proliferative disorder comprising:

1) incubating the peptide or antibody of any one of the preceding embodiments with a biological sample under conditions to allow the peptide or antibody to associate with a G-protein coupled receptor in the sample, selected from the group consisting of P2Y10, HGPRBMY3, HGPRBMY11, and HGPRBMY23; and

2) measuring levels of peptide-receptor or antibody-receptor complex formed in (1), wherein an alteration in these levels compared to standard levels indicates diagnosis of the proliferative disorder. In specific embodiments, P2Y10, HGPRBMY3, HGPRBMY11, and HGPRBMY23 comprise the amino acid sequences set forth as SEQ ID NO:112, SEQ ID NO:2, SEQ ID NO:110, and SEQ ID NO:111, respectively.

The diagnostic method of any one of the preceding embodiments, wherein the disorder is selected from the group consisting of proliferative disorders of the immune system, hematopoietic system, renal system, glandular system, reproductive system, skin or connective tissues, and gastrointestinal system.

The diagnostic method of any one of the preceding embodiments, wherein the disorder is selected from the group consisting of: leukemia and cancers of the adrenal gland, bladder, bone, bone marrow, brain, breast, cervix, colon, gall bladder, ganglia, heart, kidney, liver, lung, muscle, ovary, pancreas, parathyroid, penis, prostate, salivary gland, skin, spleen, testis, thymus, thyroid, and uterus.

The diagnostic method of any one of the preceding embodiments, wherein the disorder is selected from the group consisting of melanoma, squamous cell carcinoma, and fibrosarcoma.

A pharmaceutical composition comprising the isolated nucleic acid, vector, isolated peptide, or antibody of any one of the preceding embodiments and a physiologically acceptable carrier, excipient, or diluent.

A method of treating a proliferative disorder comprising: administering the pharmaceutical composition of any one of the preceding embodiments in an amount sufficient to treat the disorder.

The treatment method of any one of the preceding embodiments, wherein the disorder is selected from the group consisting of proliferative disorders of the immune system, hematopoietic system, renal system, glandular system, reproductive system, skin or connective tissues, and gastrointestinal system.

The treatment method of any one of the preceding embodiments, wherein the disorder is selected from the group consisting of: leukemia and cancers of the adrenal gland, bladder, bone, bone marrow, brain, breast, cervix, colon, gall bladder, ganglia, heart, kidney, liver, lung, muscle, ovary, pancreas, parathyroid, penis, prostate, salivary gland, skin, spleen, testis, thymus, thyroid, and uterus.

The treatment method of any one of the preceding embodiments, wherein the disorder is selected from the group consisting of melanoma, squamous cell carcinoma, and fibrosarcoma.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publication's, for example, Sambrook, Fritsch, and Maniatis, Molecular Cloning: a Laboratory Manual, 2^{nd} Edition, Cold Spring Harbor Laboratory Press, USA, (1989).

### Example 1

### Bioinformatics Analysis

G-protein coupled receptor sequences were used to search the Incyte and public domain EST databases with the gapped BLAST program (S. F. Altschul, et al., Nuc. Acids Res., 25:3389-4302 (1997)). The top EST hits from the BLAST results were searched back against the non-redundant protein and patent sequence databases. From this analysis, ESTs encoding potential novel GPC receptors were identified based on sequence homology. An Incyte EST (CloneID:3356166) was selected as potential novel GPC receptor candidate, called HGPRBMY3. This EST was sequenced and the full-length clone of this GPC receptor was obtained using the EST sequence information and conventional methods. The complete protein sequence of HGPRBMY3 was analyzed for potential transmembrane domains using the TMPRED program (K. Hofinann and W. Stoffel, Biol. Chem., 347:166 (1993)). The program predicted seven transmembrane domains and the predicted domains matched with the predicated transmembrane domains of related GPC receptors at the amino acid sequence level.

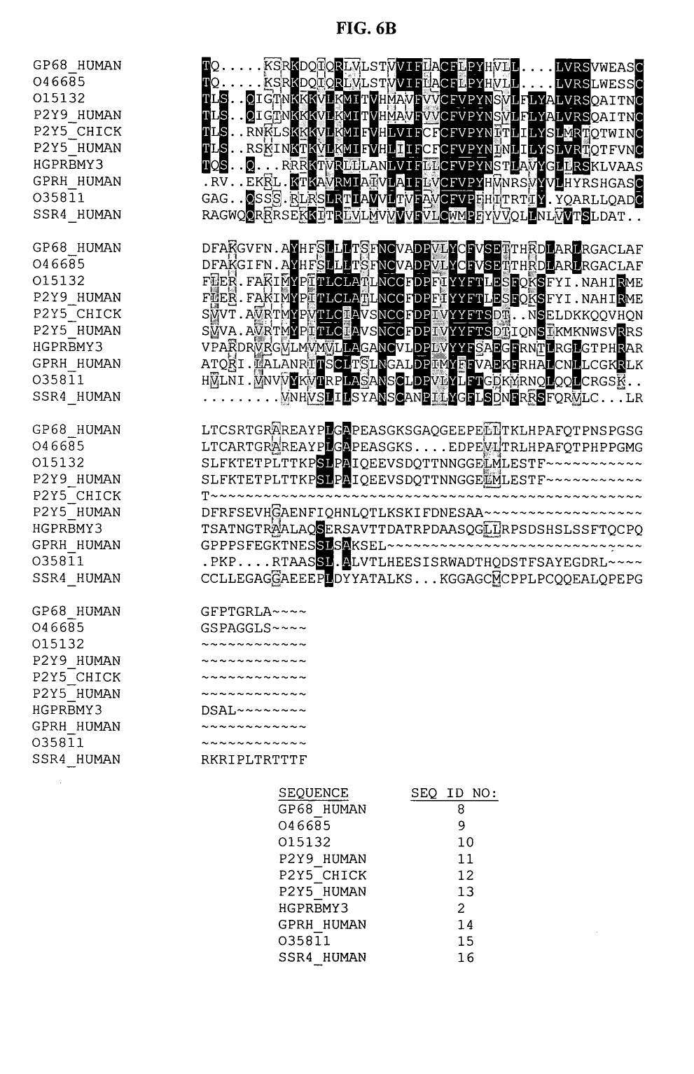

To compare HGPRBMY3 to other purinergic and somatostatin receptors, a multiple sequence alignment was generated using the GCG pileup program with (FIGS. 6A-6B). The alignment included: human HGPRBMY3 (SEQ ID NO:2); human GPR68 (GPR68_HUMAN; SEQ ID NO:8); human BRGR1B receptor (O46685; SEQ ID NO:9); human P2Y5-like receptor (O15132; SEQ ID NO:10); human P2Y9 receptor (P2Y9_HUMAN; SEQ ID NO:11); chicken P2Y5 receptor (P2Y5_CHICK; SEQ ID NO:12); human P2Y5 receptor (P2Y5_HUMAN; SEQ ID NO:13); human GPR17 receptor (GPRH_HUMAN; SEQ ID NO:14); a rat orphan GPC receptor (O35811; SEQ ID NO:15); and human SSR4 receptor (SSR4_HUMAN; SEQ ID NO:16).

Sequence analysis indicated that the HGPRBMY3 polypeptide (372 amino acids) shares 39% identity and 49.5% similarity with 284 amino acids of the chicken P2Y5 receptor (P2Y5_CHICK; GenBank Acc. No. P32250); 31.5% identity and 37.5% similarity with human GP68 receptor (GP68_HUMAN); 32.1% identity and 41.4% similarity with human GPR17 (GPRH_HUMAN; GenBank Acc. No. Q13304); 34.5% identity and 44.7% similarity with human P2Y5-like receptor (GenBank Acc. No. O15132); 35.3% identity and 40.8% similarity with a Rattus norvegicus GPC receptor (GenBank Acc. No.:O35811); 34.2% identity and 39.9% similarity with Bos taurus BRGR1B receptor (GenBank Acc. No. O46685); 35.5% identity and 46.7% similarity with human P2Y5 receptor (P2Y5_HUMAN; Acc. No.:P43657; O15133); 34.8% identity and 45% similarity with human P2Y9 receptor (P2Y9; P2Y9_HUMAN; Acc. No.:Q99677); and 31.4% identity and 38% similarity with human SS4R(SSR4_HUMAN; Acc. No.:P31391). Based on sequence, structure, and known GPC receptor motifs, HGPRBMY3 was designates as a novel, orphan human GPC receptor.

### Example 2

### Cloning of the Novel Human GPC Receptor, HGPRBMY3

Using the EST sequence, an antisense 80 bp oligonucleotide with biotin at the 5′ end (5′-b-cca agc tgt aga cca cca agt gca ggc ggt ggg tag gtc ggt agt cag gac acg gga gaa cag aac tgt tgg ttg agg ag-3′ (SEQ ID NO:5)) was designed to hybridize to the predicted coding region of HGPRBMY3. The biotinylated oligonucleotide was incubated with a mixture of single-stranded covalently closed circular cDNA libraries, which contained DNA corresponding to the sense strand. Hybrids between the biotinylated oligonucleotide and the circular cDNA were captured on streptavidin magnetic beads. Upon thermal release of the cDNA from the biotinylated oligonucleotide, the single stranded cDNA was converted into double strands using a primer homologous to a sequence on the cDNA cloning vector. The double-stranded cDNA was introduced into E. coli by electroporation. The resulting colonies were screened by PCR using a primer pair designed from the EST sequence to identify the proper cDNA. Oligonucleotides used to identify the cDNA by PCR included: HGPRBMY3s (SEQ ID NO:6; 5′-agcccaatgg cagacttgag-3′); and HGPRBMY3a (SEQ ID NO:7; 5′-ggtgcgtttg gtcacagctt-3′). Those cDNA clones that were deemed positive by PCR were analyzed to determine insert size. Two of the largest clones (7.0 Kb and 3.0 Kb) were chosen for DNA sequencing. Both clones were determined to contain identical sequences in common regions.

### Example 3

### Expression Profiling of HGPRBMY3

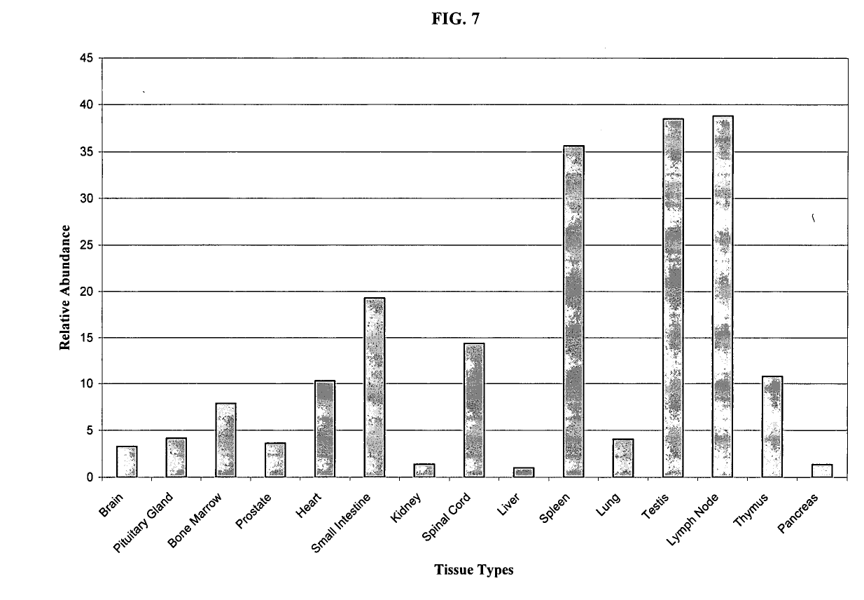

The same PCR primer pair used to identify HGPRBMY3 cDNA clones (HGPRBMY3s (SEQ ID NO:6) and HGPRBMY3a (SEQ ID NO:7)) was used to measure the steady state levels of mRNA by quantitative PCR. Briefly, first strand cDNA was made from commercially available mRNA. The relative amount of cDNA used in each assay was determined by performing a parallel experiment using a primer pair for the cyclophilin gene, which is expressed in equal amounts in all tissues. The cyclophilin primer pair detected small variations in the amount of cDNA in each sample, and these data were used for normalization of the data obtained with the primer pair for HGPRBMY3. The PCR data were converted into a relative assessment of the difference in transcript abundance among the tissues tested and the data are presented in FIG. 7. Transcripts corresponding to the orphan GPCR, HGPRBMY3, were found to be highly expressed in immune- and testes-related tissues.

### Example 4

### PCR Expression Profiling

RNA quantification was performed using the Taqman® real-time-PCR fluorogenic assay. The Taqman® assay is one of the most precise methods for assaying the concentration of nucleic acid templates. All cell lines were grown using standard conditions. Cells were grown in RPMI 1640 supplemented with 10% fetal bovine serum, 100 IU/ml penicillin, 100 mg/ml streptomycin, and 2 mM L-glutamine, 10 mM HEPES (all from Gibco BRL; Rockville, Md.). Cells at 80% confluency were washed twice with phosphate-buffered saline (Gibco BRL) and harvested using 0.25% trypsin (Gibco BRL). RNA was prepared using the RNeasy Maxi Kit from Qiagen (Valencia, Calif.). The cDNA template for real-time PCR was generated using the Superscript™ First Strand Synthesis system for RT-PCR.

SYBR Green real-time PCR reactions were prepared as follows: The reaction mix consisted of 20 ng first strand cDNA; 50 nM Forward Primer; 50 nM Reverse Primer; 0.75×SYBR Green I (Sigma); 1×SYBR Green PCR Buffer (50 mM Tris-HCl pH8.3, 75 mM KCl); 10% DMSO; 3 mM MgCl₂; 300 μM each dATP, dGTP, dTTP, dCTP; 1 U Platinum® Taq DNA Polymerase High Fidelity (Cat# 11304-029; Life Technologies; Rockville, Md.); 1:50 dilution; ROX (Life Technologies). Real-time PCR was performed using an Applied Biosystems 5700 Sequence Detection System. Conditions included incubation at 95° C. for 10 min (denaturation and activation of Platinum® Taq DNA Polymerase), and 40 cycles of incubation at 95° C. for 15 sec and 60° C. for 1 min. PCR products were analyzed for uniform melting using an analysis algorithm built into the 5700 Sequence Detection System. Forward primer: GPCR19-F2: 5′-cctggcttccacactttgtactc-3′ (SEQ ID NO:28); and Reverse primer: GPCR19-R2: 5′-tcccaacgcctctcgttct-3′ (SEQ ID NO:29).

The cDNA quantification used in the normalization of template quantity was performed using Taqman® technology. Taqman® reactions were prepared as follows. The reaction mix included 20 ng first strand cDNA; 25 nM GAPDH-F3, Forward Primer; 250 nM GAPDH-R1 Reverse Primer; 200 nM GAPDH-PVIC Taqman® Probe (fluorescent dye labeled oligonucleotide primer); 1× Buffer A (Applied Biosystems); 5.5 mM MgCl₂; 300 μM dATP, dGTP, dTTP, dCTP; and 1 U Amplitaq Gold (Applied Biosystems). GAPDH, D-glyceraldehyde-3-phosphate dehydrogenase, was used as control to normalize mRNA levels. Real-time PCR was performed using an Applied Biosystems 7700 Sequence Detection System. Conditions included incubation at 95° C. for 10 min. (denaturation and activation of Amplitaq Gold), and 40 cycles of incubation at 95° C. for 15 sec and 60° C. for 1 min. The sequences for the GAPDH oligonucleotides used in the Taqman® reactions included: GAPDH-F3 5′-agccgagccacatcgct-3′ (SEQ ID NO:30); GAPDH-R15′-gtgaccaggcgcccaatac-3′ (SEQ ID NO:31); GAPDH-PVIC Taqman® Probe VIC 5′-caaatccgttgactccgaccttcacctt-3′ TAMRA (SEQ ID NO:32).

The Sequence Detection System generated a Ct (threshold cycle) value that was used to calculate a concentration for each input cDNA template. cDNA levels for each gene of interest were normalized to GAPDH cDNA levels to compensate for variations in total cDNA quantity in the input sample. This was done by generating GAPDH Ct values for each cell line. Ct values for the gene of interest and GAPDH were inserted into a modified version of the δδCt equation (Applied Biosystems Prism® 7700 Sequence Detection System User Bulletin #2), which was used to calculate a GAPDH normalized relative cDNA level for each specific cDNA. The δδCt equation follows: relative quantity of nucleic acid template=2^{δδCt}=2^{(δCta-δCtb)}, where δCta=Ct target—Ct GAPDH, and δCtb=Ct reference—Ct GAPDH. No reference cell line was used for the calculation of relative quantity; δCtb was defined as 21.

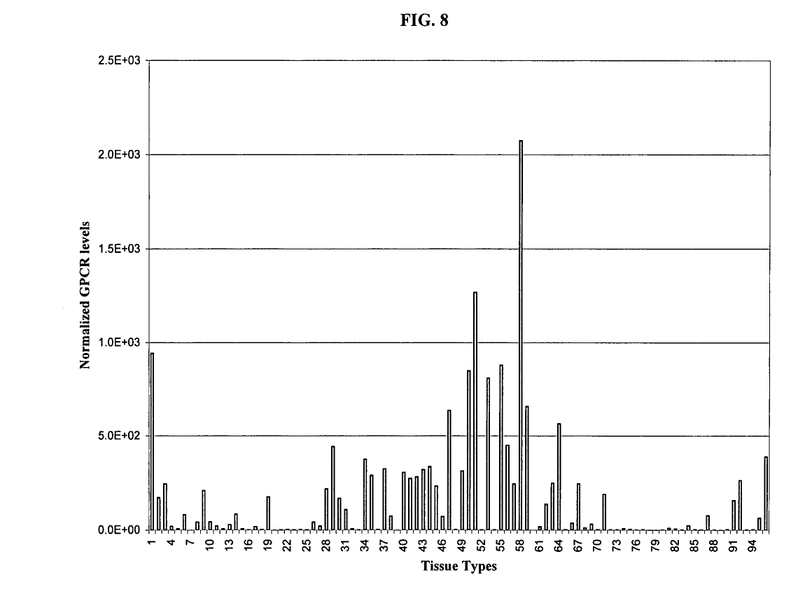

Results are shown in Table 1 and FIG. 8. Table 1 lists the cancer cell lines and transformed cell lines tested. The bar numbers in FIG. 8 are correlated to cell types in Table 1 (see column marked “Graph #”). Interestingly, HGPRBMY3 (also known as GPCR19 or GPR92) was found to be overexpressed 500- to 2000-fold greater in colon carcinoma cell lines than in cancer cell lines in the OCLP-1 (oncology cell line panel). Additionally, in some cases HGPRBMY3 was expressed at moderate to high levels in breast and ovarian tumor cell lines. This expression pattern was similar to that seen with the Lifespan analysis (Lifespan Biosciences, Inc., Seattle, Wash.) of HGPRBMY3 protein in colon, breast and ovarian tumor tissue. The Lifespan data consistently showed high expression of HGPRBMY3 protein in colon tumors and in some cases showed moderate to high expression of HGPRBMY3 in breast and ovarian tumors.

TABLE 1

| | | | | Ct | | | |
|---|---|---|---|---|---|---|---|
| | | | Ct | FPCR19- | | | |
| Graph # | Name | Tissue | GAPDH | 2 | dCt | ddCt | Quant. |
| 1 | AIN 4 | breast | 17.49 | 28.61 | 11.12 | −9.88 | 9.4E+02 |
| 2 | AIN 4T | breast | 17.15 | 30.73 | 13.58 | −7.42 | 1.7E+02 |
| 3 | AIN4/myc | breast | 17.81 | 30.88 | 13.07 | −7.93 | 2.4E+02 |
| 4 | BT-20 | breast | 17.9 | 34.62 | 16.72 | −4.28 | 1.9E+01 |
| 5 | BT-474 | breast | 17.65 | 36.1 | 18.45 | −2.55 | 5.9E+00 |
| 6 | BT-483 | breast | 17.45 | 32.13 | 14.68 | −6.32 | 8.0E+01 |
| 7 | BT-549 | breast | 17.55 | 40 | 22.45 | 1.45 | 0.0E+00 |
| 8 | DU4475 | breast | 18.1 | 33.69 | 15.59 | −5.41 | 4.3E+01 |
| 9 | H3396 | breast | 18.04 | 31.33 | 13.29 | −7.71 | 2.1E+02 |
| 10 | HBL 100 | breast | 17.02 | 32.57 | 15.55 | −5.45 | 4.4E+01 |
| 11 | Her2 MCF-7 | breast | 19.26 | 35.79 | 16.53 | −4.47 | 2.2E+01 |
| 12 | HS 578T | breast | 17.83 | 36.19 | 18.36 | −2.64 | 6.2E+00 |
| 13 | MCF 7 | breast | 17.83 | 33.96 | 16.13 | −4.87 | 2.9E+01 |
| 14 | MCF-7/AdrR | breast | 17.23 | 31.84 | 14.61 | −6.39 | 8.4E+01 |
| 15 | MDAH 2774 | breast | 16.87 | 34.95 | 18.08 | −2.92 | 7.6E+00 |
| 16 | MDA-MB-175-VII | breast | 15.72 | 37.19 | 21.47 | 0.47 | 7.2E−01 |
| 17 | MDA-MB-231 | breast | 17.62 | 34.49 | 16.87 | −4.13 | 1.8E+01 |
| 18 | MDA-MB-453 | breast | 17.9 | 37.53 | 19.63 | −1.37 | 2.6E+00 |
| 19 | MDA-MB-468 | breast | 17.49 | 31.04 | 13.55 | −7.45 | 1.7E+02 |
| 20 | Pat-21 R60 | breast | 35.59 | 40 | 4.41 | −16.59 | ND |
| 21 | SKBR3 | breast | 17.12 | 38.24 | 21.12 | 0.12 | 9.2E−01 |
| 22 | T47D | breast | 18.86 | 38.12 | 19.26 | −1.74 | 3.3E+00 |
| 23 | UACC-812 | breast | 17.06 | 39.23 | 22.17 | 1.17 | 4.4E−01 |
| 24 | AR-75-1 | breast | 15.95 | 35.4 | 19.45 | −1.55 | 2.9E+00 |
| 25 | C-33A | cervical | 17.49 | 38.9 | 21.41 | 0.41 | 7.5E−01 |
| 26 | Ca Ski | cervical | 17.38 | 32.94 | 15.56 | −5.44 | 4.3E+01 |
| 27 | HeLa | cervical | 17.59 | 34.17 | 16.58 | −4.42 | 2.1E+01 |
| 28 | HT-3 | cervical | 17.42 | 30.65 | 13.23 | −7.77 | 2.2E+02 |
| 29 | ME-180 | cervical | 16.86 | 29.07 | 12.21 | −8.79 | 4.4E+02 |
| 30 | SiHa | cervical | 18.07 | 31.68 | 13.61 | −7.39 | 1.7E+02 |
| 31 | SW756 | cervical | 15.59 | 29.83 | 14.24 | −6.76 | 1.1E+02 |
| 32 | CACO-2 | colon | 17.56 | 35.79 | 18.23 | −2.77 | 6.8E+00 |
| 33 | CCD-112Co | colon | 18.03 | 39.19 | 21.16 | 0.16 | 9.0E−01 |
| 34 | CCD-33Co | colon | 17.07 | 29.52 | 12.45 | −8.55 | 3.7E+02 |
| 35 | Colo 205 | colon | 18.02 | 30.85 | 12.83 | −8.17 | 2.9E+02 |
| 36 | Colo 320DM | colon | 17.01 | 36.06 | 19.05 | −1.95 | 3.9E+00 |
| 37 | Colo201 | colon | 17.89 | 30.55 | 12.66 | −8.34 | 3.2E+02 |
| 38 | Cx-1 | colon | 18.79 | 33.59 | 14.8 | −6.2 | 7.4E+01 |
| 39 | ddH2O | colon | 40 | 40 | 0 | −21 | ND |
| 40 | HCT116 | colon | 17.59 | 30.34 | 12.75 | −8.25 | 3.0E+02 |
| 41 | HCT116/epo5 | colon | 17.71 | 30.62 | 12.91 | −8.09 | 2.7E+02 |
| 42 | HCT116/ras | colon | 17.18 | 30.05 | 12.87 | −8.13 | 2.8E+02 |
| 43 | HCT116/TX15CR | colon | 17.36 | 30.04 | 12.68 | −8.32 | 3.2E+02 |
| 44 | HCT116/vivo | colon | 17.7 | 30.31 | 12.61 | −8.39 | 3.4E+02 |
| 45 | HCT116/VM46 | colon | 17.87 | 31.01 | 13.14 | −7.86 | 2.3E+02 |
| 46 | HCT116/VP35 | colon | 17.3 | 32.13 | 14.83 | −6.17 | 67.2E+01 |
| 47 | HCT-8 | colon | 17.44 | 29.13 | 11.69 | −9.31 | 6.3E+02 |
| 48 | HT-29 | colon | 17.9 | 37.17 | 19.27 | −1.73 | 3.3E+00 |
| 49 | LoVo | colon | 17.64 | 30.035 | 12.71 | −8.29 | 3.1E+02 |
| 50 | LS 174T | colon | 17.93 | 29.2 | 11.27 | −9.73 | 8.5E+02 |
| 51 | LS123 | colon | 17.65 | 28.34 | 10.69 | −10.31 | 1.3E+03 |
| 52 | MIP | colon | 16.92 | 37.01 | 20.09 | −0.91 | 1.9E+00 |
| 53 | SK-CO-1 | colon | 17.75 | 29.09 | 11.34 | −9.66 | 8.1E+02 |
| 54 | SW1417 | colon | 17.22 | 37.66 | 20.44 | −0.56 | 1.5E+00 |
| 55 | SW403 | colon | 18.39 | 29.61 | 11.22 | −9.78 | 8.8E+02 |
| 56 | SW480 | colon | 17 | 29.19 | 12.19 | −8.81 | 4.5E+02 |
| 57 | SW620 | colon | 17.16 | 30.23 | 13.07 | −7.93 | 2.4E+02 |
| 58 | SW837 | colon | 18.35 | 28.33 | 9.98 | −11.02 | 2.1E+03 |
| 59 | T84 | colon | 16.44 | 28.08 | 11.64 | −9.36 | 6.6E+02 |
| 60 | CCD-18Co | colon, | 17.19 | 40 | 22.81 | 1.81 | 0.0E+00 |
| | | fibroblast | | | | | |
| 61 | HT-1080 | fibrosarcoma | 17.16 | 34.03 | 16.87 | −4.13 | 1.8E+01 |
| 62 | CCRF-CEM | leukemia | 17.07 | 30.98 | 13.91 | −7.09 | 1.4E+02 |
| 63 | HL-60 | leukemia | 17.54 | 30.59 | 13.05 | −7.95 | 2.5E+02 |
| 64 | K562 | leukemia | 18.42 | 30.28 | 11.86 | −9.14 | 5.6E+02 |
| 65 | A-427 | lung | 18 | 40 | 22 | 1 | 0.0E+00 |
| 66 | A549 | lung | 17.63 | 33.38 | 15.75 | −5.25 | 3.8E+01 |
| 67 | Calu-3 | lung | 18.09 | 31.16 | 13.07 | −7.93 | 2.4E+02 |
| 68 | Calu-6 | lung | 16.62 | 34.01 | 17.39 | −3.61 | 1.2E+01 |
| 69 | ChaGo-K-1 | lung | 17.79 | 33.79 | 16 | −5 | 3.2E+01 |
| 70 | DMS 114 | lung | 18.14 | 37.5 | 19.36 | −1.64 | 3.1E+00 |
| 71 | LX-1 | lung | 18.17 | 31.61 | 13.44 | −7.56 | 1.9E+02 |
| 72 | MRC-5 | lung | 17.3 | 38.87 | 21.57 | 0.57 | 6.7E−01 |
| 73 | MSTO-211H | lung | 16.81 | 38.43 | 21.62 | 0.62 | 6.5E−01 |
| 74 | MCI-H596 | lung | 17.73 | 35.66 | 17.93 | −3.07 | 8.4E+00 |
| 75 | SHP-77 | lung | 18.66 | 37.89 | 19.23 | −1.77 | 3.4E+00 |
| 76 | Sk-LU-1 | lung | 15.81 | 37.95 | 22.14 | 1.14 | 4.5E−01 |
| 77 | SK-MES-1 | lung | 17.1 | 36.54 | 19.44 | −1.56 | 2.9E+00 |
| 78 | SW1721 | lung | 16.45 | 40 | 23.55 | 2.55 | 0.0E+00 |
| 79 | SW1573 | lung | 17.14 | 40 | 22.86 | 1.86 | 0.0E+00 |
| 80 | SW900 | lung | 18.17 | 40 | 21.83 | 0.83 | 0.0E+00 |
| 81 | Hs 294T | melanoma | 17.73 | 35.42 | 17.69 | −3.31 | 9.9E+00 |
| 82 | A2780/DDP-R | ovarian | 21.51 | 40 | 18.49 | −2.51 | 0.0E+00 |
| 83 | A2780/DDP-S | ovarian | 17.89 | 40 | 22.11 | 1.11 | 0.0E+00 |
| 84 | A2780/epo5 | ovarian | 17.54 | 33.94 | 16.4 | −4.6 | 2.4E+01 |
| 85 | A2780/TAX-R | ovarian | 18.4 | 38.49 | 20.09 | −0.91 | 1.9E+00 |
| 86 | A2780/TAX-S | ovarian | 17.83 | 38.67 | 20.84 | −0.16 | 1.1E+00 |
| 87 | Caov-3 | ovarian | 15.5 | 30.23 | 14.73 | −6.27 | 7.7E+01 |
| 88 | ES-2 | ovarian | 17.22 | 38.34 | 21.12 | 0.12 | 9.2E−01 |
| 89 | HOC-76 | ovarian | 34.3 | 40 | 5.7 | −15.3 | 0.0E+00 |
| 90 | OVCAR-3 | ovarian | 17.09 | 37.09 | 20 | −1 | 2.0E+00 |
| 91 | PA-1 | ovarian | 17.33 | 31.05 | 13.72 | −7.28 | 1.6E+02 |
| 92 | SW626 | ovarian | 16.94 | 29.91 | 12.97 | −8.03 | 2.6E+02 |
| 93 | UPN251 | ovarian | 17.69 | 39.07 | 21.38 | 0.38 | 7.7E+01 |
| 94 | LNCAP | prostate | 18.17 | 38.5 | 20.33 | −0.67 | 1.6E+00 |
| 95 | PC-3 | prostate | 17.25 | 32.27 | 15.02 | −5.98 | 6.3E+01 |
| 96 | A431 | squamous | 19.85 | 32.25 | 12.4 | −8.6 | 3.9E+02 |

### Example 5

### Functional Characterization of HGPRBMY3

### DNA Constructs

The HGPRBMY3 cDNA was PCR amplified using PFU™ (Stratagene). The primers used in the PCR reaction were specific to the HGPRBMY3 polynucleotide and were ordered from Gibco BRL. The primers included: 5′ primer (SEQ ID NO:33) 5′-gtccccaagc ttgcaccatg ttagccaaca gctcctcaac caacagttct-3′; and 3′ primer (SEQ ID NO:34) 5′-gtccgcggat ccctacttgt cgtcgtcgtc cttgtagtcc atgagggcgg aatcctgggg acactgtgtgaa-3′. The 5′ primer contained a HindIII site at the 5′ end. The 3′ primer contained a BamHI site at the 5′ end, a Kozak sequence, and a sequence encoding the FLAG™ epitope tag. The product from the PCR reaction was isolated from a 0.8% Agarose gel (Invitrogen) and purified using a Gel Extraction Kit™ (Qiagen). The purified product was then digested overnight along with the pcDNA3.1 Hygro™ mammalian expression vector from Invitrogen using the Hind III and BamHI restriction enzymes (New England Biolabs). The digested products were then purified using the Gel Extraction Kit™ (Qiagen) and subsequently ligated to the pcDNA3.1 Hygro™ expression vector using a DNA molar ratio of 4:1 for insert:vector. All DNA modification enzymes were purchased from NEB. The ligation was incubated overnight at 16° C., after which time, 1 μl of the mix was used to transform DH5-alpha cloning efficiency competent E. coli™ (Gibco BRL). A detailed description of the pcDNA3.1 Hygro™ mammalian expression vector is available at the Invitrogen web site (world wide web.Invitrogen.com). The plasmid DNA from the ampicillin-resistant clones were isolated using the Wizard DNA Miniprep System from Promega. Positive clones were then confirmed and scaled-up for purification using the Qiagen Maxiprep™ plasmid DNA purification kit.

### Cell Line Generation

The pcDNA3.1 Hygro vector containing HGPRBMY3 cDNA was used to transfect CHO-NFAT/CRE or CHO-NFAT/Gα15 (Aurora Biosciences) cells using Lipofectamine 2000™ according to the manufacturer's instructions (Gibco BRL). NFAT is known as the nuclear factor activator of transcription, and CRE is known as the cAMP response element. Two days later, the cells were split 1:3 into selective media (DMEM 11056, 600 μg/ml Hygromycin, 200 μg/ml Zeocin, 10% fetal bovine serum, FBS). All cell culture reagents were purchased from Gibco BRL-Invitrogen. The CHO-NFAT/CRE or CHO-NFAT/Gα15 cell lines, transiently or stably transfected with HGPRBMY3, were analyzed using FACS Vantage SE™ (BD), fluorescence microscopy (Nikon), and LJL Analyst™ (Molecular Devices). This system was used to determine changes in real-time gene expression as a consequence of constitutive G-protein coupling of HGPRBMY3. Changes in gene expression were determined by the fluorescence emission of the transformed cells at 447 nm and 518 nm, and visualized using beta-lactamase as a reporter.

Beta-lactamase hydrolyzes an intracellularly loaded, membrane-permeant ester substrate, cephalosporin-coumarin-fluorescein2/acetoxymethyl (CCF2/AM™; Aurora Biosciences), when induced by an appropriate signaling cascade. The CCF2/AM™ substrate is a 7-hydroxycoumarin cephalosporin with a fluorescein attached through a stable thioether linkage. With this substrate, induced expression of the beta-lactamase enzyme is readily apparent, as each enzyme molecule produced is capable of changing the fluorescence of many CCF2/AM™ substrate molecules. A schematic of this cell based system is shown below. For the intact molecule, excitation of the coumarin at 409 nm results in fluorescence resonance energy transfer (FRET) to the fluorescein that emits green light at 518 nm. Production of active beta-lactamase results in cleavage of the beta-lactam ring, leading to the disruption of FRET, and excitation of the coumarin only. This gives rise to blue fluorescent emission at 447 nm. Additional details relative to the cell loading methods and/or instrument settings may be found by reference to the following publications: Zlokarnik, et al., 1998; Whitney et al., 1998; and BD Biosciences,1999 (see above).

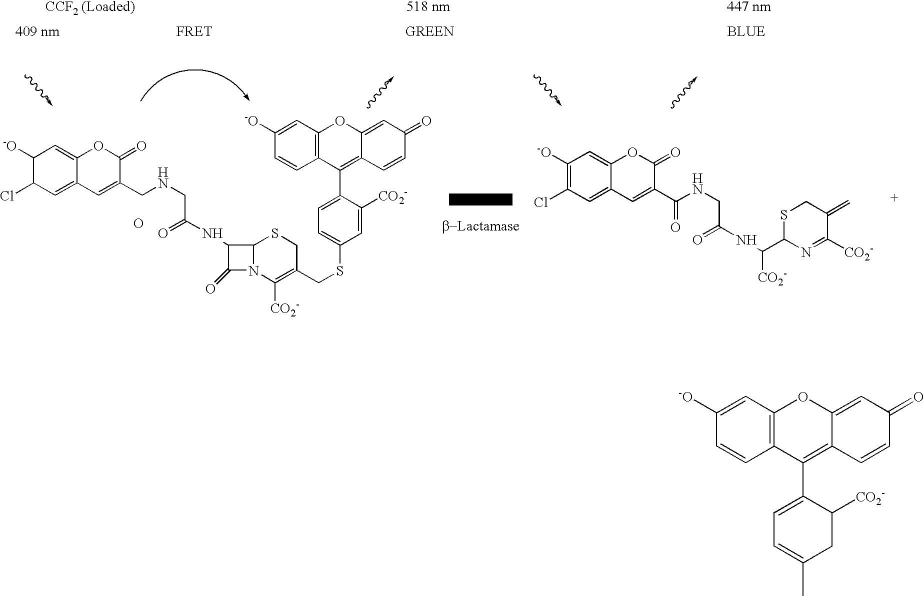

Fluorescence emissions were detected using a Nikon-TE300 microscope equipped with an excitation filter (D405/10X-25), dichroic reflector (430DCLP), and a barrier filter for dual DAPI/FITC (510 nm) to visually capture changes in beta-lactamase expression. The FACS Vantage SE was equipped with a Coherent Enterprise II Argon Laser and a Coherent 302C Krypton laser. The optical filters on the FACS Vantage SE™ were HQ460/50m and HQ535/40m bandpass separated by a 490 dichroic mirror. Prior to analyzing the fluorescent emissions from the cell lines as described above, the cells were loaded with the CCF2/AM^{TM} substrate. A 6×CCF2/AM™ loading buffer was prepared, and 1 mM CCF2/AM™ (Aurora Biosciences) was dissolved in 100% DMSO (Sigma). Stock solution (12 μl) was added to 60 μl of 100 mg/ml Pluronic F127 (Sigma) in DMSO containing 0.1% acetic acid (Sigma). This solution was added while vortexing to 1 ml of Sort Buffer (PBS minus calcium and magnesium-Gibco-25 mM HEPES-Gibco-pH 7.4, 0.1% BSA). Cells were placed in serum-free media and the 6×CCF2/AM was added to a final concentration of 1×. The cells were then loaded at room temperature for 1-2 h, and then subjected to fluorescent emission analysis as described herein.

### Immunocytochemistry and FACS Analysis

There is strong evidence that certain GPC receptors exhibit cDNA concentration-dependent constitutive activity as measured by cAMP response element (CRE) luciferase reporters (Chen et al., 1999). In an effort to demonstrate functional coupling of HGPRBMY3 to known GPC receptor second messenger pathways, the HGPRBMY3 polypeptide was expressed at high constitutive levels in the CHO-NFAT/CRE cell line. To this end, the HGPRBMY3 cDNA was PCR amplified and subcloned into the pcDNA3.1 Hygro™ mammalian expression vector as described herein. Early passage CHO-NFAT/CRE cells were then transfected with the resulting pcDNA3.1 Hygro™/HGPRBMY3 construct.

Transfected CHO-NFAT/CRE cells were analyzed by immunocytochemistry to confirm expression of the FLAG® & tag. The cells were plated at 1×10³ in each well of a glass slide (VWR). The cells were rinsed with PBS, followed by acid fixation for 30 min at room temperature using a mixture of 5% glacial acetic acid and 90% ethanol. The cells were then blocked in 2% BSA and 0.1% Triton in PBS, and incubated for 2 h at room temperature, or overnight at 4° C. A monoclonal anti-FLAG® FITC antibody was diluted to 1:50 in blocking solution, and incubated with the cells for 2 h at room temperature. Cells were then washed three times with 0.1% Triton in PBS for 5 min. The slides were overlaid with mounting media dropwise with Biomedia-Gel Mount™ (Biomedia; containing anti-quenching agent). Cells were examined at 10× magnification using the Nikon TE300 equipped with FITC filter (535 nm).

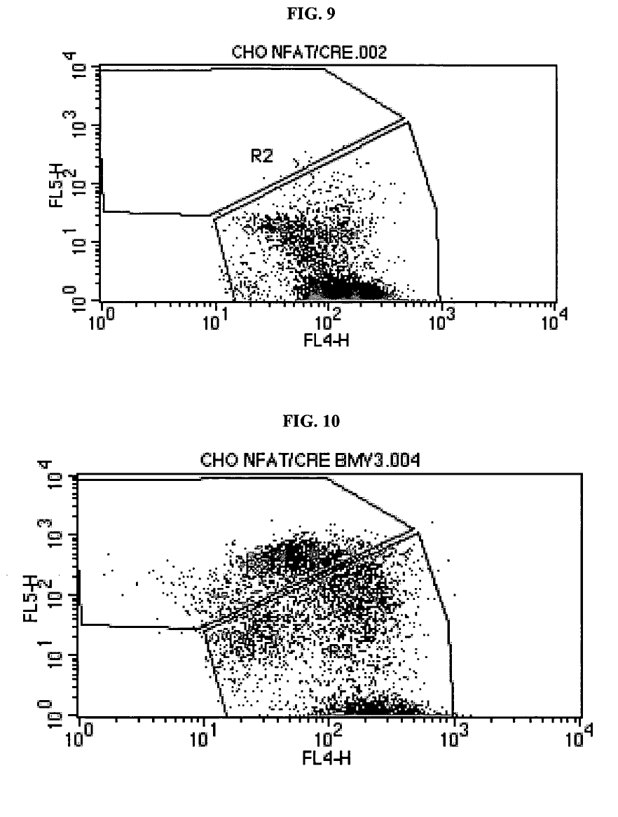

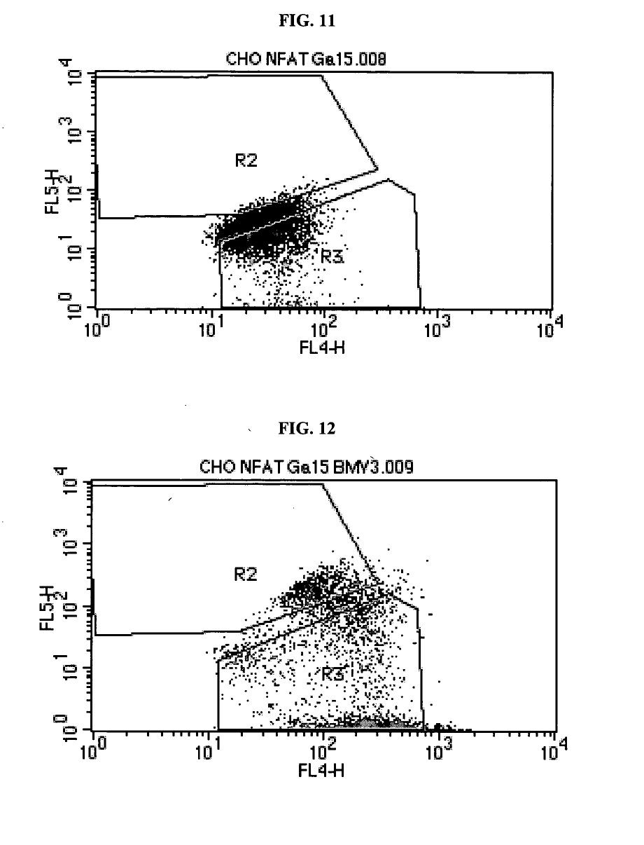

Transfected and non-transfected (control) CHO-NFAT/CRE cells were then analyzed for fluorescence emissions by FACS (fluorescence-activated cell sorting). Cells were loaded with the CCF2 substrate and stimulated with 10 nM PMA, 1 μM thapsigargin (NFAT stimulator), and 10 μM forskolin (CRE stimulator) to fully activate the NFAT/CRE element. Analysis of wavelength emissions at 518 nM (channel R3; green) and 447 nM (channel R2; blue) was performed. A significant number of CHO-NFAT/CRE cells transfected with pcDNA3.1 Hygro™/HGPRBMY3 showed fluorescent emissions at 447 nm (FIG. 10), as compared to the non-transfected controls (FIG. 9). This demonstrated the constitutive activity of HGPRBMY3 in the CHO-NFAT/CRE line, and showed that overexpression of HGPRBMY3 produced functional coupling and subsequent activation of beta-lactamase gene expression. Most of the control cells showed emissions at 518 nm, with few emissions observed at 447 nm (FIG. 9). This was expected since the NFAT/CRE response elements remain dormant in the absence of an activated G-protein dependent signal transduction pathway (e.g., pathways mediated by Gq/11 or Gs receptors).

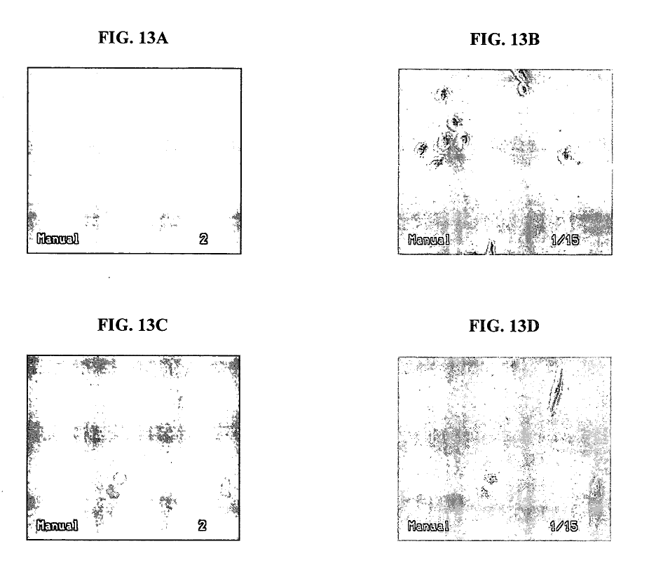

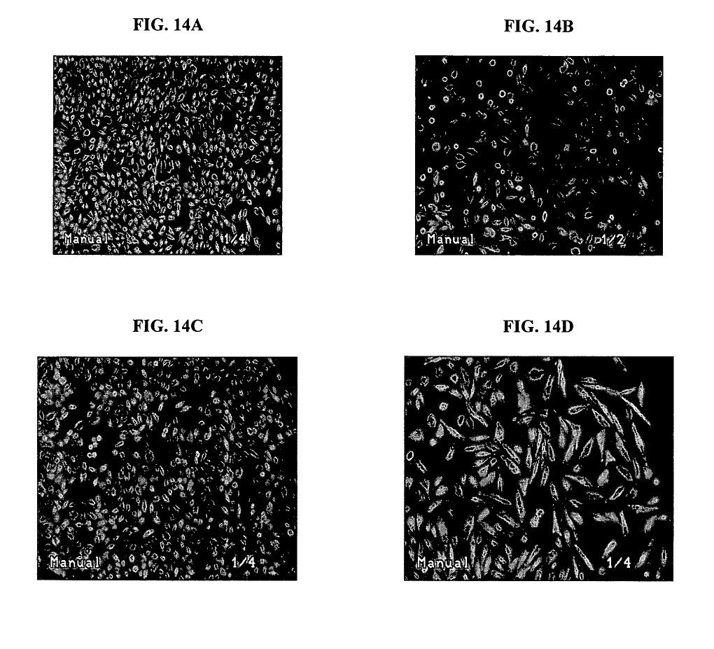

Additional experiments were performed to characterize the functional coupling of HGPRBMY3. For these experiments, the promiscuous G-protein, Gα15 was utilized. It was previously shown that the C-terminal 20 amino acids of either Gα15 or 16 confer the unique ability of these G proteins to couple to many GPC receptors, including those that naturally do not stimulate phospholipase C, PLC (Blahos et al., 2001). To demonstrate that HGPRBMY3 was functioning as a GPC receptor, the CHO-NFAT/Gα15 cell line was transfected with the pcDNA3.1 Hygro™/HGPRBMY3 construct. The cells were analyzed via FACS according to their wavelength emission at 518 nm (channel R3; green), and 447 nm (channel R2; blue). A significant number of CHO-NFAT/Gα15 cells transfected with pcDNA3.1 Hygro™/HGPRBMY3 showed fluorescent emissions at 447 nm (FIG. 12), as compared to the non-transfected controls (FIG. 11). This demonstrated the constitutive activity of HGPRBMY3 in the CHO-NFAT/Gα15 line, and showed that overexpression of HGPRBMY3 produced functional coupling and subsequent activation of beta-lactamase gene expression. Most of the control cells showed emissions at 518 nm, with few emissions observed at 447 nm (FIG. 11). These results indicated that HGPRBMY3 represents a functional GPC receptor analogous to known Gα15-coupled receptors. It was concluded that constitutive expression of HGPRBMY3 in the CHO-NFAT/Gα15 cell line leads to NFAT activation through accumulation of intracellular Ca²⁺, as previously demonstrated for the M3 muscarinic receptor (Boss et al., 1996).

### Demonstration of Cell Surface Expression

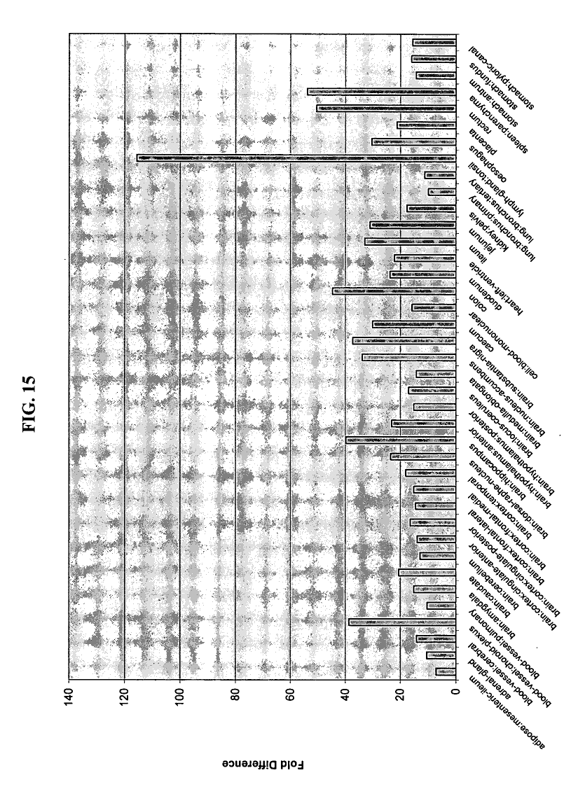

HGPRBMY3 was tagged using the FLAG® epitope, and the tagged sequence was inserted into the pcDNA3.1 Hygro™ expression vector, as described herein. CHO-NFAT/Gα15 cells transfected with the FLAG®-tagged HGPRBMY3 were analyzed by immunocytochemistry. Briefly, transfected cells were fixed with 70% methanol and permeablized with 0.1% Triton X-100. Cells were blocked with 1% serum and incubated with a FITC-conjugated monoclonal antibody raised against the FLAG® epitope tag at 1:50 dilution in PBS-Triton. Cells were then washed several times with PBS-Triton, overlayed with mounting solution, and fluorescent images were captured. Cells were viewed by bright field and fluorescence microscopy (emissions at 530 nm following illumination with a mercury light source). The non-transfected control cell line exhibited no detectable background fluorescence (FIG. 13A). The cell line transfected with HGPRBMY3-FLAG® exhibited plasma membrane expression of the tagged protein (FIG. 13C), consistent with the observations for other GPC receptors. Taken together, the data indicate that HGPRBMY3 is a cell surface GPC receptor that can function through increases in Ca²⁺ signal transduction pathways via Gα15.

### Screening Paradigm

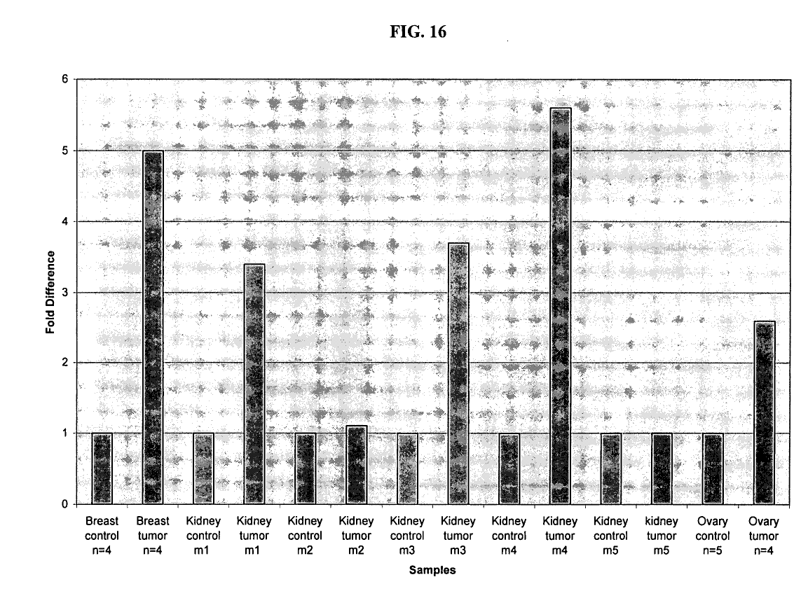

To allow the identification of modulators (e.g., agonists and antagonists) of HGPRBMY3, cell lines were constructed to exhibit a range of constitutive coupling activity. Coupling activity was measured using beta-lactamase technology (Aurora) and the FACS Vantage SE™ system (BD). CHO-NFAT/CRE cell lines were transfected with pcDNA3.1 Hygro™/HGPRBMY3 and isolated via FACS based on intermediate or high beta-lactamase expression levels of constitutive activation. FIG. 14A shows CHO-NFAT/CRE cells with pcDNA3.1 Hygro™/HGPRBMY3 prior to stimulation with 10 nM PMA, 1 μM thapsigargin, and 10 μM forskolin (−P/T/F). FIG. 14B shows CHO-NFAT/CRE cells with pcDNA3.1 Hygro™/HGPRBMY3 after stimulation with 10 nM PMA, 1 μM thapsigargin, and 10 μM forskolin (+P/T/F). FIG. 14C shows CHO-NFAT/CRE cells transfected with the HGPRBMY3 orphan GPC receptor (oGPCR) that generates intermediate levels of beta-lactamase expression after stimulation with P/T/F. FIG. 14D shows CHO-NFAT/CRE transfected with the HGPRBMY3 oGPCR that generates high levels of beta-lactamase expression after stimulation with P/T/F.

The identification of cell lines with an intermediate level of GPC receptor expression, and an intermediate coupling response, provides the opportunity to screen, indirectly, for both agonists and antagonists of HGPRBMY3 by looking for inhibitors that block the beta-lactamase response, or agonists that increase the beta-lactamase response. As described herein, the expression level of beta-lactamase directly correlates with the level of cleaved CCF2 substrate. This paradigm has previously been used to identify modulators of 5HT6, a known GPC receptor that couples through adenylate cyclase. Similar screens have also been used to identify modulators of the 5HT2c, a GPC receptor that couples through changes in [Ca ²⁺]i.

Screens to identify HGPRBMY3 modulators may be carried out using a variety of high-throughput methods known in the art, though preferred screens employ a fully-automated UHTSS system (Aurora). For such screens, the uninduced CHO-NFAT/CRE cell line carrying pcDNA3.1 Hygro™/HGPRBMY3 can be used to determine the relative background level of beta-lactamase expression (FIG. 14A). Following treatment with a cocktail of 10 nM PMA, 1 μM thapsigargin, and 10 μM forskolin (FIG. 14B), the cells pcDNA3.1 Hygro™/HGPRBMY3 fully activate the CRE-NFAT response element, and can be used to determine the dynamic range of the assay.

### Example 6

### Expression Profiling Using Expanded Tissue and Cell Sources

### Probes and Primers

Total RNA from tissues was isolated using the TriZol protocol (Invitrogen) and quantified by determining its absorbance at 260 μm. An assessment of the 18s and 28s ribosomal RNA bands was made by denaturing gel electrophoresis to determine RNA integrity. The specific sequence to be measured was aligned with related genes found in GenBank to identity regions of significant sequence divergence to maximize primer and probe specificity. Gene-specific primers and probes were designed using the ABI primer express software to amplify small amplicons (150 bp or less) to maximize the likelihood that the primers functioned at maximum efficiency. All primer/probe sequences were searched against public GenBank databases to ensure target specificity. Primers and probes were obtained from ABI. For HGPRBMY3, the primer probe sequences included: Forward Primer 5′-ccagcggtgggaagtgat-3′ (SEQ ID NO:64); Reverse Primer 5′-caaaggcatttcgtcctcttct-3′ (SEQ ID NO:65); and TaqMan Probe 5′-ccctctgcacgggtgggctct-3′ (SEQ ID NO:66).

### DNA Contamination

To access the level of contaminating genomic DNA, the RNA was divided into 2 aliquots and one-half was treated with Rnase-free Dnase (Invitrogen). Samples from both the Dnase-treated and non-treated aliquots were used for reverse transcription reactions with (RT+) and without (RT−) reverse transcriptase. TaqMan assays were carried out with gene-specific primers (see above), and the contribution of genomic DNA to the signal detected was evaluated. For these evaluations, the threshold cycles of RT+/RT− non-Dnase treated RNA were compared to those of RT+/RT− Dnase treated RNA. The amount of signal contributed by genomic DNA in the Dnased RT− RNA was required to be less that 10% of that obtained with Dnased RT+RNA. Otherwise, the RNA was not used for further experiments.

### Reverse Transcription Reaction and Sequence Detection

For the reactions, 100 ng of Dnase-treated total RNA was annealed to 2.5 μM of the respective gene-specific reverse primer in the presence of 5.5 mM MgCl₂ by heating the sample to 72° C. for 2 min and then cooling to 55° C. for 30 min. Next, 1.25 U/μl of MuLv reverse transcriptase and 500 μM of each dNTP was added to the reaction, and the tube was incubated at 37° C. for 30 min. The sample was then heated to 90° C. for 5 min to denature the enzyme. Quantitative sequence detection was carried out on an ABI PRISM 7700 system. For detection, the reverse transcribed reaction was mixed with 2.5 μM forward and reverse primers, 2.0 μM of the TaqMan probe, 500 μM of each dNTP, buffer, and 5 U AmpliTaq Gold™. The PCR reaction included incubation at 94° C. for 12 min, followed by 40 cycles of incubation at 94° C. for 15 sec and 60° C. for 30 sec.

### Data Handling

The threshold cycle (Ct) of the lowest expressing tissue (the highest Ct value) was used as the baseline of expression. All other tissues were expressed as the relative abundance to that tissue by calculating the difference in Ct value between the baseline and the other tissues, and using it as the exponent in 2^{(ΔCt).}

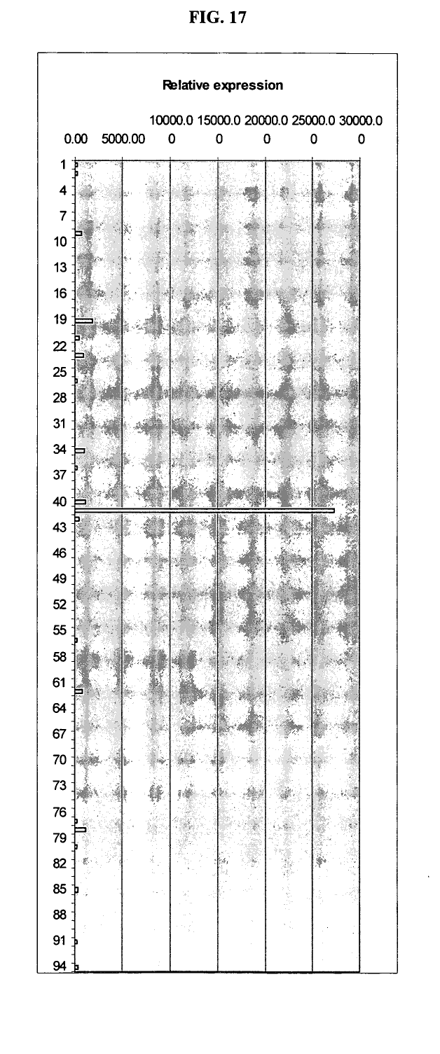

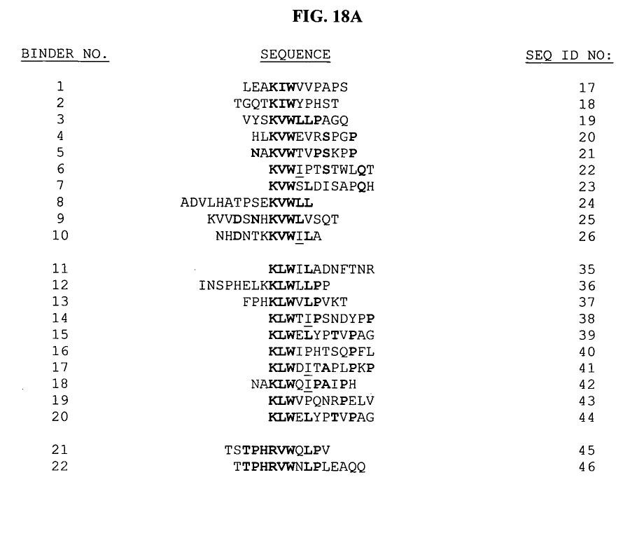

The results of these experiments are shown in FIGS. 15 and 16. Expanded analysis of HGPRBMY3 expression levels confirmed that the HGPRBMY3 polypeptide is expressed in lymph node, and spleen (FIG. 15). HGPRBMY3 mRNA was expressed in a majority of the normal tissues tested with predominate expression observed in lymph gland (tonsil). Significant expression was also observed in spleen, rectum, colon, brain, blood vessel, and to a lesser extent in other tissues as shown. An additional analysis of HGPRBMY3 expression levels in disease cells and tissues indicated that the HGPRBMY3 polypeptide is differentially expressed in kidney cancer, breast cancer, and ovary cancer tissue compared to each respective normal tissue (FIG. 16). These data support a role of HGPRBMY3 in regulating various cell-cycle functions, particularly in kidney, breast, and ovary tissues. Modulators of HGPRBMY3 function may therefore represent a novel therapeutic option for the treatment of proliferative diseases, including renal, reproductive, and and/or other proliferative disorders.

### Example 7

### Expression Profiling in Cancer Cell Lines

RNA quantification was performed using the Taqman® real-time-PCR fluorogenic assay. PCR primer pairs were designed to the specific gene and used to measure the steady state levels of mRNA by quantitative PCR across a panel of RNA's isolated from proliferative cell lines. All cell lines were grown using standard conditions. Media included RPMI 1640 supplemented with 10% fetal bovine serum, 100 IU/ml penicillin, 100 mg/ml streptomycin, and 2 mM L-glutamine, 10 mM HEPES (all from Gibco BRL; Rockville, Md.). At 80% confluency, cells were washed twice with phosphate-buffered saline (Gibco BRL) and harvested using 0.25% trypsin (Gibco BRL). RNA was prepared using the RNeasy Maxi Kit from Qiagen (Valencia, Calif.). Briefly, first strand cDNA was made from several cell line RNA's and subjected to real time quantitative PCR using a PE 7900HT instrument (Applied Biosystems, Foster City, Calif.) which detects the amount of DNA amplified during each cycle by the fluorescent output of SYBR green, a DNA binding dye specific for double stranded DNA.

The specificity of the primer pairs for their targets was verified by performing a thermal denaturation profile at the end of the run. The profile gave an indication of the number of different DNA sequences present through determination of the melting temperature of the double stranded amplicon(s). In this experiment, only one DNA fragment of the correct Tm was detected, having a homogeneous melting point. Small variations in the amount of cDNA used in each tube were accounted for by performing parallel experiments. The parallel reactions utilized a primer pair for cyclophilin, a gene expressed in equal amounts in all tissues. These data were used to normalize the data obtained with the gene specific primer pairs. The PCR data was converted into a relative assessment of the difference in transcript abundance amongst the tissues tested and the data are presented in bar graph form for each transcript. The formula for calculating the relative abundance is: Relative abundance=2^{-ΔΔCt}; where ΔΔCt=(The Ct of the sample—the Ct for cyclophilin)—the Ct for a calibrator sample. The calibrator sample is arbitrarily chosen as the tissue with the lowest abundance.

For each PCR reaction, 10 μl of 2×SYBR green master mix (PE Biosystems) was combined with 4.9 μl water, 0.05 μl of each PCR primer (at 100 μM concentration), and 5 μl of template DNA. The PCR reactions included incubation at 95° C. for 10 min, followed by 40 cycles of incubation at 95° C. for 30 seconds and 60° C. for 1 min, followed by a thermal denaturation protocol at 60° C. The fluorescence was measured as the temperature was slowly increased to 95° C. The PCR primers included: Forward Primer 5′-ccagcggtgggaagtgat-3′ (SEQ ID NO:64); and Reverse Primer 5′-caaaggcatttcgtcctcttct-3′ (SEQ ID NO:65).

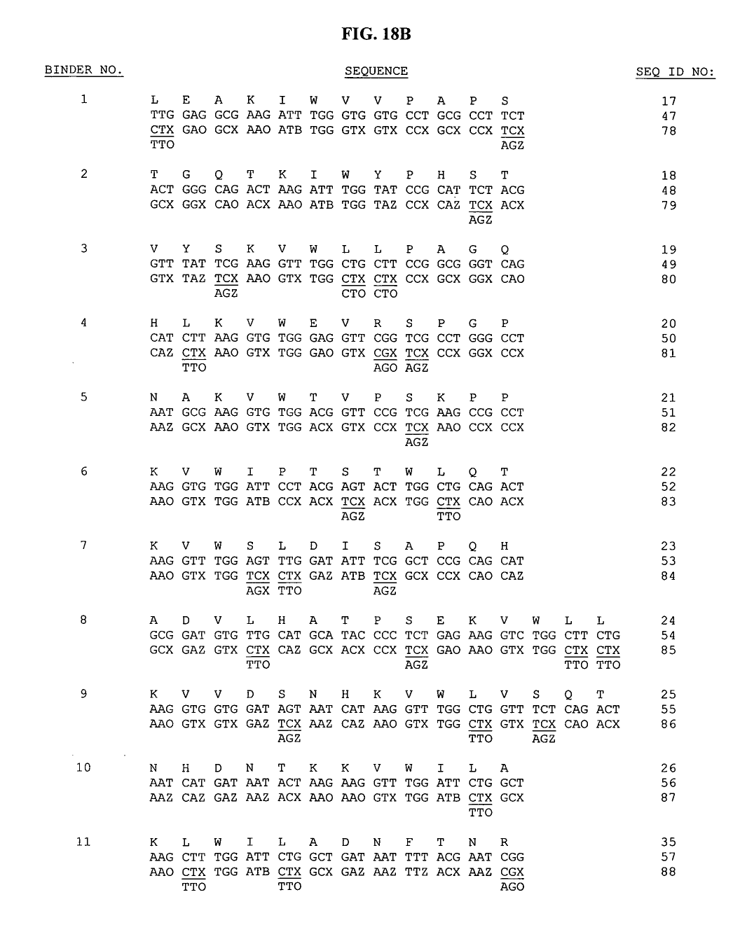

Results are shown in Table 2 and FIG. 17. Table 2 lists the cancer cell lines and transformed cell lines tested. The bar numbers in FIG. 17 are correlated to cell types in Table 2 (see columns marked “Graph #”). The results indicated that HGPRBMY3 is expressed in leukemia cell lines, significantly in cervical, melanoma, and ovarian cancer cell lines, and to a lesser extent in other human tumor cell lines (FIG. 17; Table 2). This is consistent with HGPRBMY3 having a general role in modulating cell cycle regulation, either directly or indirectly, and thus proliferative potential. The observed expression of HGPRBMY3 in normal tissues (FIG. 17; Table 2) suggested that it plays a significant role in cell cycle regulation in immune cells and tissues, particularly those of the hematopoetic system, such as B-cells, T-cells, lymph nodes, and spleen. The sum of the expression data suggested the HGPRBMY3 polypeptide may play a critical role in the development of a transformed phenotype leading to the development of cancers and/or other proliferative conditions, either directly or indirectly. Alternatively, the HGPRBMY3 polypeptide may play a protective role and could be activated in response to a cancerous or proliferative phenotype. Whether HGPRBMY3 plays a role in directing transformation, or plays the role of protecting cells in response to a transformed phenotype, its role in leukemia, cervical cancer, melanoma, and/or ovarian tumors is likely to be enhanced relative to normal tissues. Therefore, antagonists or agonists of the HGPRBMY3 polypeptide may be useful in the treatment, amelioration, and/or prevention of a variety of proliferative conditions, including, but not limited to leukemia, cervical cancer, melanoma, and/or ovarian cancer.

TABLE 2

| Graph | | | Fold |
|---|---|---|---|
| # | Name | Tissue | Difference |
| 1 | AIN4 | breast | 183.56 |
| 2 | AIN4/myc | breast | 202.75 |
| 3 | AIN4T | breast | 171.58 |
| 4 | BT-20 | breast | 14.20 |
| 5 | BT-474 | breast | 14.44 |
| 6 | BT-549 | breast | 91.21 |
| 7 | DU4475 | breast | 58.22 |
| 8 | H3396 | breast | 50.28 |
| 9 | HBL100 | breast | 742.05 |
| 10 | MCF7 | breast | 152.75 |
| 11 | BCF-7/AdrR | breast | 54.54 |
| 12 | MCF7/Her2 | breast | 23.67 |
| 13 | MDA-MB-175-VII | breast | 1.95 |
| 14 | MDA-MB-231 | breast | 19.59 |
| 15 | C-33A | cervical | 4.77 |
| 16 | Ca Ski | cervical | 165.46 |
| 17 | HeLa | cervical | 33.59 |
| 18 | HT-3 | cervical | 50.89 |
| 19 | ME-180 | cervical | 1907.50 |
| 20 | SiHa | cervial | 98.45 |
| 21 | SW756 | cervical | 437.62 |
| 22 | CACO-2 | colon | 24.15 |
| 23 | Colo201 | colon | 903.73 |
| 24 | HCT116 | colon | 78.33 |
| 25 | HCT116/epo5 | colon | 100.91 |
| 26 | HCT116/ras | colon | 193.34 |
| 27 | HCT116/TX15CR | colon | 96.91 |
| 28 | HCT116/vivo | colon | 30.32 |
| 29 | HCT116/VM46 | colon | 98.83 |
| 30 | HCT116/VP35 | colon | 18.00 |
| 31 | HT-29 | colon | 4.92 |
| 32 | LoVo | colon | 169.65 |
| 33 | LS 174T | colon | 101.68 |
| 34 | SK-CO-1 | colon | 1071.09 |
| 35 | SW480 | colon | 66.13 |
| 36 | SW620 | colon | 199.10 |
| 37 | HUVEC | endothelial | 15.77 |
| 38 | NCI-N87 | gastric | 49.73 |
| 39 | CCRF-CEM | leukemia | 53.80 |
| 40 | HL-60 | leukemia | 1133.60 |
| 41 | Jurkat | leukemia | 27333.98 |
| 42 | K-562 | leukemia | 422.13 |
| 43 | A-427 | lung | 1.03 |
| 44 | A549 | lung | 58.50 |
| 45 | Calu-3 | lung | 98.62 |
| 46 | Calu-6 | lung | 3.72 |
| 47 | ChaGo-K-1 | lung | 28.35 |
| 48 | DMS 114 | lung | 7.56 |
| 49 | LX-1 | lung | 110.19 |
| 50 | SHP-77 | lung | 7.57 |
| 51 | Sk-LU-1 | lung | 1.00 |
| 52 | SK-MES-1 | lung | 3.46 |
| 53 | SW1271 | lung | 8.66 |
| 54 | SW1573 | lung | 5.04 |
| 55 | SW900 | lung | 7.50 |
| 56 | total RNA, | lung | 252.03 |
| fetal lung | | | |
| 57 | A-375 | melanoma | 50.63 |
| 58 | C32 | melanoma | 7.79 |
| 59 | G-361 | melanoma | 6.61 |
| 60 | Hs 249T | melanoma | 7.19 |
| 61 | SK-MEL-1 | melanoma | 70.19 |
| 62 | SK-MEL-28 | melanoma | 804.95 |
| 63 | SK-MEL-3 | melanoma | 3.18 |
| 64 | SK-MEL-5 | melanoma | 3.55 |
| 65 | WM373 | melanoma | 33.54 |
| 66 | MW852 | melanoma | 4.72 |
| 67 | A2780/DDP-R | ovarian | 2.60 |
| 68 | A2780/DDP-S | ovarian | 2.31 |
| 69 | A2780/epo5 | ovarian | 7.45 |
| 70 | A2780/TAX-R | ovarian | 11.15 |
| 71 | A2780/TAX-S | ovarian | 1.72 |
| 72 | Caov-3 | ovarian | 23.40 |
| 73 | ES-2 | ovarian | 6.21 |
| 74 | HOC-76 | ovarian | 6.19 |
| 75 | OVCAR-3 | ovarian | 42.01 |
| 76 | PA-1 | ovarian | 24.87 |
| 77 | SW626 | ovarian | 257.75 |
| 78 | total RNA, ovary | ovarian | 1217.14 |
| 79 | 22Rv-1 | prostate | 28.82 |
| 80 | CA-HPV-10 | prostate | 183.82 |
| 81 | DU 145 | prostate | 5.27 |
| 82 | LNCAP | prostate | 12.17 |
| 83 | LNCaP-FGC | prostate | 4.60 |
| 84 | PC-3 | prostate | 46.14 |
| 85 | PWR-1E | prostate | 297.25 |
| 86 | RWPE-1 | prostate | 87.68 |
| 87 | PWPE-2 | prostate | 54.37 |
| 88 | RPMI-2650 | SCC | 11.21 |
| 89 | SCC-15 | SCC | 14.55 |
| 90 | SCC-25 | SCC | 51.19 |
| 91 | SCC-4 | SCC | 217.64 |
| 92 | SCC-9 | SCC | 27.93 |
| 93 | HS804.SK | skin | 10.73 |
| 94 | A-431 | squamous | 318.11 |

### Example 8

### Peptides that Bind to HGPRBMY3

### Creation of Peptide Libraries

To identify HGPRBMY3 binders, two types of libraries were created: i) libraries of 12- and 15-mer peptides, used to find peptides that may function as (ant-)agonists and ii) libraries of peptides with 23, 27, or 33 random residues, used to find natural ligands through database searches. The 15-mer library was constructed at Bristol-Myers Squibb using the M13KE vector (New England Biolabs) and a single-stranded library oligonucleotide extension method (S. S. Sidhu et al., Methods Enzymol., 2000, 328:333-363). The 12-mer library was obtained as an aliquot of the M13KE-based ‘PhD’ 12-mer library (New England Biolabs). The libraries with 23, 27, or 33 random residues were constructed at Bristol-Myers Squibb in vector M13KE (New England Biolabs) using the method described in (S. S. Sidhu et al., Methods Enzymol., 2000, 328:333-363). All libraries in the M13KE vector utilized the standard NNK motif to encode the specified number of random residues, where N=A+G+C+T and where K=G+T. For screening, mixtures of the libraries were used, including Mix 1: 12-mer library and 15-mer library; and Mix 2: 23-mer library, 27-mer library, and 33-mer library.

### Panning Method

To minimize cell lysis, especially during the multiple washes, top speeds were not exceeded for centrifugation. For eppendorf centrifuges, spins were carried out at a maximum of 3K for 30 sec. For refrigerated benchtop centrifuges, spins were performed to reach 3K, then stopped. Two days prior to panning, CHO-K1 cells were transfected with DNA encoding HGPRBMY3 (pcDNA3.1 Hygro™-HGPRBMY3 construct described in Example 5) using standard procedures. Cells were checked for sufficient expression 48 h post transfection. Sufficient cells were grown to produce a pellet corresponding to ˜50 μl volume in an eppendorf tube (˜10⁶-10⁷ cells). Typically, this corresponded to one P175 flask with near-confluent growth. A similar number of parental cells were grown for preadsorption. One day prior to panning, two or three 96-well Immulon plates were coated integrin. Plates were coated overnight at 4° C. in NaHCO₃, pH 9.5, with ˜50 ng of αVβ3 integrin (Chemicon; Cat. # CC 1020) per well per library.

On the day of panning, growth medium was discarded and cells were washed with 10 ml PBS by allowing the buffer to flow over the cells. The PBS was removed and 10 ml Tris-EDTA detaching buffer (Gibco Cat. # 13150-016; no trypsin) was added for 2 min. Plates were tapped and/or pipetted up and down to detach cells. This was done quickly to minimize the exposure of the cells to this reagent. Cells were pelleted by centrifugation at 3K, and then washed with 20 ml PBS. Cells were resuspended cells in PBS with 2% milk blocking agent plus protease inhibitor (EDTA-free; Roche Cat. # 1 873 580). For each library, cell suspensions of ˜500 μl were used. Cells were blocked for 30-60 min with gentle rocking at room temperature. The integrin-coated wells were washed 3×in PBST, then blocked with 2% BSA in PBS for 30 min or more. To preadsorb against integrin, input phage was added to the integrin-coated wells, and incubated for 30 min or more.

At this stage, the blocked parental cells were divided into the required number of aliquots (500 μl aliquot/library). The phage supernatants were added from the integrin-preadsorption step. Preadsorption against the parental cells was carried out for 30 min or more with gentle rocking. The blocked transfected cells were divided into the required number of aliquots (500 μl aliquot per library). The transfected cells were pelleted, and the supernatant was discarded. The phage supernatants from the two preadsorption steps were added, and cells were incubated with gentle rocking for 2 h or more. Cells were washed 6-8× with PBST at 5 min intervals. Each wash was performed by gently pipetting the cells up and down, and cells were centrifuged at low speed. To recover binding phage from the washed cell pellets, 500 μl 6M urea, pH 3.0 was added for 15 min. This was neutralized with 10 μl 2 M Tris (not adjusted for pH). The phage in the eluate were titered and arnplified by standard procedures (NEB protocol for PhD phage libraries). In some cases, the eluate was viscous due to the presence of chromosomal DNA.

### Sequencing of Bound Phage

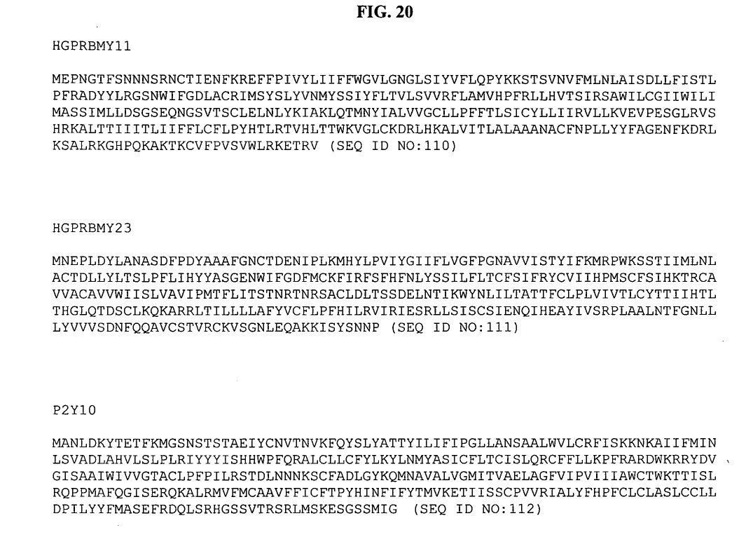

Standard procedures were used. Phage in eluates were infected into E. coli host strain ER2738 (New England Biolabs) for all M13KE-based libraries, and cells were plated for plaques. Colonies were grown in liquid medium and analyzed by standard sequencing procedures. For sequencing, PCR products were generated with suitable primers (Primer 96 from NEB: 5′-gat aaa ccg ata caa tta aag gct α-3′ (SEQ ID NO:72)) that annealed adjacent to the library segments in the vectors, and the PCR products were sequenced using one primer of each PCR primer pair. Sequences were analyzed for homologies by visual inspection or by using the Vector NTI alignment tool. The sequence analysis identified 22 peptide binders, and several binding motifs, including KIW, KVW, KLW, and RVW (FIG. 18A). Notably, none of the triplet motifs were identified in peptides that bound to parental cells. In contrast, peptides with these triplet motifs were recovered from panning with other known and predicted P2Y-type or related GPC receptors (P2Y10, HGPRBMY11 (U.S. Ser. No. 09/991,225, filed Nov. 16, 2001, which is hereby incorporated by reference in its entirety), and HGPRBMY23 (U.S. Ser. No. 10/010,568, filed Dec. 7, 2001, which is hereby incorporated by reference in its entirety), which show sequence similarity to HGPRBMY3 (FIGS. 6A-6B; FIGS. 19A-19B; and FIG. 20).

### Peptide Synthesis

Peptides were synthesized on Fmoc-Knorr amide resin (N-(9-fluorenyl)methoxycarbonyl-Knorr amide-resin, Midwest Biotech, Fishers, Indiana) using an Applied Biosystems model 433A synthesizer (Applied Biosystems, Foster City, Calif.) and the FastMoc chemistry protocol (0.25 mmol scale) supplied with the instrument. Amino acids were double-coupled as their N-alpha-Fmoc-derivatives and reactive side chains were protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group was removed by the multi-step treatment with piperidine in N-Methylpyrrolidone described by the manufacturer. The N-terminal free amines were then treated with 10% acetic anhydride, 5% Diisopropylamine in N-Methylpyrrolidone to yield the N-acetyl-derivative. The protected peptidyl-resins were simultaneously deprotected and removed from the resin by standard methods. The lyophilized peptides were purified on C₁₈ to apparent homogeneity as judged by RP-HPLC analysis. Predicted peptide molecular weights were verified by electrospray mass spectrometry (J. Biol. Chem. vol. 273, pp.12041-12046, 1998).

The contents of all patents, patent applications, published PCT applications and articles, books, references, reference manuals, abstracts, in addition to the Sequence Listing, cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. An isolated nucleic acid selected from the group consisting of:
(a) an isolated nucleic acid encoding an amino acid sequence selected from the group consisting of SEQ ID NO:17-SEQ ID NO:26, SEQ ID NO:35-SEQ ID NO:46, and SEQ ID NO:73-SEQ ID NO:77;
(b) An isolated nucleic acid selected from the group consisting of SEQ ID NO:47-SEQ ID NO:63, SEQ ID NO:67-SEQ ID NO:71, SEQ ID NO:78-SEQ ID NO:99, SEQ ID NO:100-SEQ ID NO:104, and SEQ ID NO:105-SEQ ID NO:109;
(c) an isolated nucleic acid comprising at least 20 contiguous nucleotides of a nucleotide sequence selected from the group consisting of: SEQ ID NO:47-SEQ ID NO:63, SEQ ID NO:67-SEQ ID NO:71, SEQ ID NO:78-SEQ ID NO:99, SEQ ID NO:100-SEQ ID NO:104, and SEQ ID NO:105-SEQ ID NO:109;
(d) an isolated nucleic acid comprising a nucleotide sequence that is at least 56% identical to the nucleotide sequence provided in (a), (b), or (c);
(e) an isolated nucleic acid comprising a nucleotide sequence that is complementary to the nucleotide sequence provided in (a), (b), (c), or (d).

2. A vector comprising the isolated nucleic acid provided in claim 1.

3. A host cell comprising the vector of claim 2, wherein the host cell is selected from the group consisting of bacterial, yeast, insect, mammalian, and plant cells.

4. A primer comprising the isolated nucleic acid of claim 1.

5. A probe comprising the isolated nucleic acid of claim 1.

6. An isolated peptide comprising an amino acid sequence selected from the group consisting of:
(a) an isolated peptide comprising an amino acid sequence selected from the group consisting of: SEQ ID NO:17-SEQ ID NO:26, SEQ ID NO:35-SEQ ID NO:46, and SEQ ID NO:73-SEQ ID NO:77;
(b) an isolated peptide comprising at least 8 contiguous amino acids of an amino acid sequence selected from the group consisting of SEQ ID NO:17-SEQ ID NO:26, SEQ ID NO:35-SEQ ID NO:46, and SEQ ID NO:73-SEQ ID NO:77;
(c) an isolated peptide comprising an amino acid sequence that is at least 84% identical to the amino acid sequence provided in (a), or (b).

7. A polypeptide complex comprising a P2Y-type G-protein coupled receptor, and a peptide comprising an amino acid sequence selected from the group consisting of SEQ ID NO:17-SEQ ID NO:26 AND SEQ ID NO:35-SEQ ID NO:46, and SEQ ID NO:73-SEQ ID NO:77.

8. A polypeptide complex comprising a G-protein coupled receptor selected from the group consisting of P2Y10, HGPRBMY3, HGPRBMY11, and HGPRBMY23, and a peptide comprising an amino acid sequence selected from the group consisting of SEQ ID NO:17-SEQ ID NO:26, SEQ ID NO:35-SEQ ID NO:46, and SEQ ID NO:73-SEQ ID NO:77.

9. An antibody that binds to the isolated peptide of claim 6.

10. The antibody according to claim 9 wherein said antibody is selected from the group consisting of:
(a) a monoclonal antibody;
(b) a polyclonal antibody; and
(c) an anti-idiotype antibody;
(d) an anti-idiotype antibody raised to the monoclonal antibody (a), which binds to a P2Y-type G-protein coupled receptor;
(e) an anti-idiotype antibody raised to the monoclonal antibody of (a), which binds to a G-protein coupled receptor selected from the group consisting of P2Y10, HGPRBMY3, HGPRBMY11, and HGPRBMY23;
(f) an antibody that binds to the polypeptide complex of claim 7; and
(g) an antibody that binds to the polypeptide complex of claim 8.

11. A method of isolating a peptide comprising an amino acid sequence selected from the group consisting of SEQ ID NO:17-SEQ ID NO:26 SEQ ID NO:35-SEQ ID NO:46, and SEQ ID NO:73-SEQ ID NO:77 comprising the steps of:
incubating the antibody of claim 10 with the peptide, thereby isolating the peptide.

12. A peptide library generated from the isolated nucleic acid of claim 1.

13. A method of identifying a binding agent for a P2Y-type G-protein coupled receptor comprising: screening the peptide library of claim 12 for one or more peptides that bind to a G-protein coupled receptor, wherein binding indicates identification of an G-protein coupled receptor-binding agent.

14. A method of identifying a binding agent for a G-protein coupled receptor selected from the group consisting of P2Y10, HGPRBMY3, HGPRBMY11, and HGPRBMY23, comprising: screening the peptide library of claim 12 for one or more peptides that bind to P2Y10, HGPRBMY3, HGPRBMY11, or HGPRBMY23, wherein binding indicates identification of a binding agent.

15. A method of identifying a P2Y-type G-protein coupled receptor comprising:
(a) incubating a member of the group consisting of: the isolated peptide of claim 6; and the antibody of claim 9; with a biological sample under conditions that allow the peptide or antibody to bind to a G-protein coupled receptor in the sample, and thereby form a complex; and
(b) identifying formation of the complex in (a), wherein formation of the complex indicates identification of a G-protein coupled receptor.

16. A method of identifying binding agent for a P2Y-type G-protein coupled receptor comprising:
(a) incubating the isolated polypeptide complex of claim 7 with a test agent under conditions that allow the test agent to bind to the complex; and
b) screening for disruption of the polypeptide complex, wherein disruption of the complex indicates identification of a binding agent.

17. A method of identifying binding agent for a P2Y-type G-protein coupled receptor comprising:
(a) incubating a G-protein coupled receptor with a test agent;
(b) incubating the receptor and test agent with the isolated peptide of claim 6; and
(c) screening for formation of a polypeptide complex between the receptor and the isolated peptide, wherein inhibition of formation of the complex indicates identification of a binding agent.

18. A kit for detecting a P2Y-type G-protein coupled receptor comprising:
(a) a member of the group consisting of: the isolated peptide of claim 6;
and the antibody of claim 9; and
(b) one or more reagents for detecting binding of the receptor and the peptide or antibody.

19. A method of diagnosing a proliferative disorder comprising:
(a) incubating a member of the group consisting of: the isolated peptide of claim 6, and the antibody of claim 9; with a biological sample under conditions to allow said peptide or said antibody to associate with a P2Y-type G-protein coupled receptor in the sample; and
(b) measuring levels of said peptide-receptor or said antibody-receptor complex formed in (a), wherein an alteration in these levels compared to standard levels indicates diagnosis of the proliferative disorder.

20. A pharmaceutical composition comprising a member of the group consisting of: the isolated nucleic acid of claim 1; the isolated vector of claim 2, the isolated peptide of claim 6, and the antibody of claim 9; and a physiologically acceptable carrier, excipient, or diluent.

21. A method of treating a proliferative disorder comprising: administering the pharmaceutical composition of claim 20 in an amount sufficient to treat the disorder.
